(19) United States
(12) Patent Application Publication
(10) Pub. No.: US 2026/0177560 A1
(43) Pub. Date: Jun. 25, 2026

(54) BIOMARKER PANEL FOR BRAIN SPECIFIC ABNORMAL NEUROLOGICAL CONDITIONS USING BIOFLUID SAMPLES

(71) Applicant: GRYPHON BIO, INC., South San Francisco, CA (US)

(72) Inventors: William E. Haskins, South San Francisco, CA (US); Devin Jackson, South San Francisco, CA (US)

(73) Assignee: GRYPHON BIO, INC., South San Francisco, CA (US)

(21) Appl. No.: 19/129,782

(22) PCT Filed: Nov. 16, 2023
(86) PCT No.: PCT/US2023/080001

§ 371 Date: May 14, 2025

Related U.S. Application Data
(60) Provisional application No. 63/425,761, filed on Nov. 16, 2022.

(51) Int. Cl. G01N 33/68

(52) U.S. Cl. CPC G01N 33/6893; G01N 33/6848; G01N 33/6896

(57) ABSTRACT

A process for determining an extent of a central nervous system (CNS) specific neurological condition in a subject including collecting a biological sample of biofluid from the subject and measuring a quantity of a first biomarker, or metabolite of or mRNA corresponding to, the first biomarker from the sample from a dried spot or through a microfluidic device. The biofluid is capillary blood or saliva, which affords ease of collection advantages that are attractive for field-, hospital-, and home-based environments. The process being useful in the diagnosis, care, and management of brain specific abnormal neurological conditions in general, and in particular, to traumatic brain injury (TBI) and (TBI-induced) Alzheimer's disease (AD) and Alexander disease, in which a GFAP mutation is implicated in white matter deterioration.

### RELATED APPLICATIONS

This application claims priority benefit of U.S. Provisional Application Ser. No. 63/425,761 filed 16 Nov. 2022; the contents of which are hereby incorporated by reference.

### FIELD OF THE INVENTION

The present invention relates in general to determination of a brain or central nervous system (CNS) specific abnormal neurological condition of an individual such as a brain/CNS injury and in particular to measuring a quantity of brain specific neuropredictive conditional biomarkers in a biofluid sample of saliva or capillary blood (wet and dried blood, dried serum, or dried plasma samples) from finger prick blood collection, alone or in combination with additional biomarkers from plasma or serum from venous blood collection, to detect, monitor, diagnose, prognosticate, predict, differentiate, aid in the treatment of the abnormal condition, or a combination thereof. For optimal clinical utilities, biomarkers in samples from biofluid, and combinations with additional biomarkers in samples from venous or arterial blood, are most ideally measured at multiple time points following brain/CNS injury event or brain/CNS disorder initiation in field-, hospital-, and home-based environments.

### BACKGROUND OF THE INVENTION

The field of clinical neurology remains frustrated by the recognition that secondary injury to a central nervous system tissue associated with physiologic response to the initial insult could be lessened if only the initial insult could be rapidly diagnosed or in the case of a progressive disorder before stress on central nervous system tissues reached a preselected threshold. Traumatic, ischemic, and neurotoxic chemical insult, along with generic disorders, all present the prospect of brain damage. While the diagnosis of severe forms of each of these causes of brain damage is straightforward through clinical response testing and computed tomography (CT) and magnetic resonance imaging (MRI) testing, these diagnostics have their limitations in that spectroscopic imaging is both costly and time consuming while clinical response testing of incapacitated individuals is of limited value and often precludes a nuanced diagnosis. Additionally, owing to the limitations of existing diagnostics, situations under which a subject experiences a stress to their neurological condition such that the subject often is unaware that damage has occurred or seek treatment as the subtle symptoms often quickly resolve. The lack of treatment of these mild to moderate challenges to neurologic condition of a subject can have a cumulative effect or subsequently result in a severe brain damage event which in either case has a poor clinical prognosis.

There is also a growing appreciation that rapid intervention once a TBI is detected can greatly improve outcomes. The ability for a first responder to detect a TBI provides optimal clinical opportunities to limit the secondary inflammatory cascade that follows the injury. Increasing evidence suggests that TBI is also a risk factor for the development of age-associated neurodegenerative disorders including Alzheimer's Disease (AD) and Parkinson's Disease (PD). (Dams-O'Connor, K. et al.; Duan, Y. et al.; Lee, P. C. et al.; Sivanandam, T. M. et anon). Moderate to severe TBI has been shown in autopsy studies to result in increased amyloid and microtubule associated protein tau (Tau) deposition in the brain. Tau is a neuronal protein which helps stabilize microtubules in the axon. Tau is phosphorylated (P-Tau) at many sites potentially by cellular protein kinases. Especially of interests are P-Tau phosphorylated at Thr181, Ser202 & Thr231 and S396/404. (Duan, Y. et al.) as well as P-Tau phosphorylated at Thr217 (Thijssen, La Joie et al. 2021, Mielke, Aakre et al. 2022, Mielke, Dage et al. 2022). Elevated levels P-Tau are seen in the brain in chronic traumatic encephalopathy for years following mild TBI or concussion. (McKee, A. C. et al.; and Omalu, B. I. et al.).

In order to overcome the limitations associated with spectroscopic and clinical response diagnosis of neurological condition, there is increasing attention on the use of biomarkers as internal indicators of change as to molecular or cellular level health condition of a subject. As detection of biomarkers uses a sample obtained from a subject and detects the biomarkers in that sample, typically cerebrospinal fluid, blood, or plasma, biomarker detection holds the prospect of inexpensive, rapid, and objective measurement of neurological condition. With the attainment of rapid and objective indicators of neurological condition allows one to determine severity of a non-normal brain condition on a scale with a degree of objectivity, predict outcome, guide therapy of the condition, as well as monitor subject responsiveness and recovery. Additionally, such information as obtained from numerous subjects allows one to gain a degree of insight into the mechanism of brain injury.

A number of biomarkers have been identified as being associated with severe traumatic brain injury as is often seen in vehicle collision and combat wounded subjects. Understanding how multiple biomarkers overlap and any correlations to injury severity remains unestablished. This lack of understanding is particularly prevalent with respect to traumatic injuries to the brain.

Analyses of a blast injury to a subject produced several inventive correlations between proteins and neuronal injury as an illustrative neurological condition. Neuronal injury is optionally the result of whole body blast, blast force to a particular portion of the body, or the result of other neuronal trauma or disease that produces detectable or differentiable levels of neuroactive biomarkers. Thus, identifying pathogenic pathways of primary blast brain injury (BBI) in reproducible experimental models is vital to the development of diagnostic algorithms for differentiating severe, moderate and mild (mTBI) from posttraumatic stress disorder (PTSD). Accordingly, a number of experimental animal models have been implemented to study mechanisms of blast wave impact and include rodents and larger animals such as sheep. However, because of the rather generic nature of blast generators used in the different studies, the data on brain injury mechanisms and putative biomarkers have been difficult to analyze and compare.

In spite of the extensive survey of brain specific proteins or autoantibodies thereto that become systemic in response to a brain specific abnormal neurological condition, usage of such markers has met with limited success in the field owing to a variety of issues such as sensitivity and the ability to obtain results in a clinically timely fashion.

The requirement for venous blood draws as a method of sample collection to detect biomarkers of abnormal neurological condition that are elevated in blood limits the usage of such tests. As a result, care and/or treatment relevant data is either not available or delayed. As care and/or treatment of many abnormal neurological conditions has a limited timing window in order to avoid secondary injury, this has proven to be problematic.

Thus, there exists a need for a process and an assay for providing improved measurement of brain specific abnormal neurological condition based on a biofluid of a subject, such as saliva or blood drawn from the capillaries of a subject, and further need for a sample obtained by a finger prick. There also exists a need for a detection process and assay amenable to correlating a saliva, dried blood, serum, or plasma spot sampled from biofluid for an abnormal neurological condition with conventional venous or arterial blood drawn samples.

### SUMMARY OF THE INVENTION

The present invention provides a process for determining an extent of a central nervous system (CNS) specific neurological condition in a subject that includes collecting a biological sample of biofluid from the subject and measuring a quantity of a first biomarker, or metabolite of or mRNA corresponding to, the first biomarker from the sample from a dried spot or through a microfluidic device. The biofluid is capillary blood or saliva, which affords ease of collection advantages that are attractive for field-, hospital-, and home-based environments. The present invention has utility in the diagnosis, care, and management of brain specific abnormal neurological conditions in general, and in particular, to traumatic brain injury (TBI) and (TBI-induced) Alzheimer's disease (AD) and Alexander disease, in which a GFAP mutation is implicated in white matter deterioration.

### BRIEF DESCRIPTION OF THE DRAWINGS

FIG. 1A depicts a kit suitable for capillary blood biofluid draw according to the present invention;

FIG. 1B shows a dried plasma spot (DPS) sampling and immunoassay with exemplary biomarkers shown as a function of concentration profile relative to clinical events and the cellular injury cascade with each temporal peak for a biomarker correlated with an exemplary cellular source of the biomarker;

FIG. 1C shows a TBI temporal biomarker platform solution and workflow according to embodiments of the present invention with dry and wet serial plasma sample collection from a TBI patient that are then processed and analyzed at a centralized testing lab for the selected TBI temporal biomarker panel results to be reported to hospital, physician, and/or to the patients;

FIG. 2 is a graph showing high frequency dried plasma spot (DPS) sampling and high sensitivity immunoassays of a proprietary panel of blood based temporal TBI biomarkers as a single platform solution (SPS);

FIG. 3A is a plot of median/IQR of pilot data for longitudinal wet serum third NF-L versus days post injury;

FIG. 3B is a graph showing median/IQR pf NF-L Ratio to D1 versus days post injury;

FIG. 3C is a box-and-whisker plot showing NF-L levels for a healthy control, one day post injury, 20 days post injury, and six months post injury;

FIG. 3D is a box-and-whisker plot showing pNF-H levels for a healthy control, one day post injury, 20 days post injury, and six months post injury;

FIG. 4A shows localization of MOG at the external lamellae of myelin sheaths;

FIG. 4B shows Serum MOG elevations in acute and subacute-chronic TBI samples. Median comparisons (Kruskal-Wallis test, ***P<0.001 compared to control or ##, p<0.01;

FIG. 4C shows chronic time course of serum MOG showing the temporal profile for 12 patients;

FIG. 4D and FIG. 4E show mean levels (and range) of anti-MOG antibody IgG and IgM, respectively at D1, 2 week and 6 month post-injury, as compared to those of healthy controls. *p<0.01, **p<0.05 different from healthy control IgG and p<0.05 different form Day 1 TBI;

FIG. 4F and FIG. 4G show the MOG antibody IgG and IgM levels, respectively expressed as a ratio of the patients own day 1 ratio. Red dotted lines show the lack of change form day 1 base line as ratio remains one. A subset of TBI patients show 2-fold to more than 20-fold increase of IgG or IgM by 2 week or 6 month post-injury;

FIG. 4H shows that when MOG IgG and IgM levels (as ratio of D1 levels) over time were subjected to unsupervised trajectory analysis, three hidden trajectory classes were identified. Class 1 is declining trajectory, Class 2 is flat trajectory, while class 3 is elevated or increased trajectory;

FIG. 5A is a plot of median/IQR for serum total Tau pilot data temporal profile;

FIG. 5B is a plot of median/IQR for serum pTau (231) pilot data temporal profile;

FIG. 5C is a plot of median/IQR for serum p-Tau (181) pilot data temporal profile;

FIG. 5D is a plot of median/IQR for serum pTau (231) pilot data temporal profile;

FIG. 5E is a plot of median/IQR for serum P-Tau (181) pilot data temporal profile;

FIG. 6A shows pilot data on the high sensitivity first immunoassay platform showing the synaptic marker VAMP5 at elevated levels in TBI patients 2 weeks and 6 months following TBI, as compared to Healthy controls and TBI D1. *p<0.05, ANOVA of means; n=5-7;

FIG. 6B shows pilot data on the high sensitivity first immunoassay platform showing the neuronal cell body marker WASF1) at elevated levels in TBI patients 2 weeks and 6 months following TBI, as compared to Healthy controls and TBI D1. *p<0.05, ANOVA of means; n=5-7;

FIG. 7 is a graph showing pilot serum VEGF-A levels in the days after a TBI and in a healthy control, notably, VEGF peaks in days 7-10, which is later than p-TAU but earlier than NF-L and NF-H;

FIG. 8 is a graph IL-6 levels in days following a TBI and in a healthy control, notably IL-6 levels in CSF and serum are elevated from day 0-7 after severe TBI. IL-6 assay was by sandwich ELISA (R&D systems);

FIG. 9A is a graph showing preliminary verification of the analytical comparability of a first high sensitivity immunoassay platform vs. a second conventional immunoassay platforms with NFL measurements of de-identified, archived wet plasma specimens from full spectrum TBI subjects (1 dpi to 6 months) with the first NFL data showing a very strong correlation with second NFL data (R2=0.861);

FIG. 9B is a graph showing preliminary verification of the analytical comparability of the first high sensitivity immunoassay platform vs. third immunoassay platforms with NFL measurements of de-identified, archived wet plasma specimens from full spectrum TBI subjects (1 dpi to 6 months) with the First NFL data showing a good correlation with third NFL data (R2=0.656);

FIG. 10A is a graph showing preliminary verification of the analytical comparability of wet vs. DPS for sampling for NFL and WASF1 measurements of de-identified, archived plasma specimens from full spectrum TBI subjects (1 day after injury to 6 months) with the First high sensitivity immunoassay platform;

FIG. 10B is a graph showing preliminary verification of the analytical comparability of wet vs. DPS for sampling for WASF1 measurements of de-identified, archived plasma specimens from full spectrum TBI subjects (1 day after injury to 6 months) with the First high sensitivity immunoassay platform;

FIG. 11A is a graph showing preliminary verification of the analytical comparability of wet vs. DPS for sampling with respect to the recovery of TBI biomarkers with a serial dilution of recombinant protein biomarker GFAP spanning five orders of magnitude for a spiking recovery study in pooled plasma from 10 healthy controls age 40-45;

FIG. 11B is a graph showing preliminary verification of the analytical comparability of wet vs. DPS for sampling with respect to the recovery of TBI biomarkers with a serial dilution of recombinant protein biomarker NFL spanning five orders of magnitude for a spiking recovery study in pooled plasma from 10 healthy controls age 40-45;

FIG. 11C is a graph showing preliminary verification of the analytical comparability of wet vs. DPS for sampling with respect to the recovery of TBI biomarkers with a serial dilution of recombinant protein biomarker Tau spanning five orders of magnitude for a spiking recovery study in pooled plasma from 10 healthy controls age 40-45;

FIG. 11D is a graph showing preliminary verification of the analytical comparability of wet vs. DPS for sampling with respect to the recovery of TBI biomarkers with a serial dilution of recombinant protein biomarker UCH-L1 spanning five orders of magnitude for a spiking recovery study in pooled plasma from 10 healthy controls age 40-45;

FIG. 12 shows the levels of wet-bio source MOG at various testing points over time;

FIG. 13 shows results of a Kruskal-Wallis test with Serum MBP elevations in a control group, TBI day 0-10, and TBI day 12 to 12 month samples with significant different from controls (***p<0.0001) and showing that delayed MBP blood elevations is similar to the NF-L pattern;

FIG. 14A is a scatter plot of serum GFAP over 14 days post injury with a normative baseline of 60 pg/mL;

FIG. 14B is a scatter plot of serum GFAP ratio to D1 over 14 days post injury;

FIG. 15 shows the levels of wet plasma GFAP (third platform) at various testing points over time;

FIG. 16A shows the levels of wet plasma NFL (First platform) at various testing points over time;

FIG. 16B shows the levels of wet plasma NFL (second platform) at various testing points over time;

FIG. 16C shows the levels of wet plasma NFL (third platform) at various testing points over time;

FIG. 17A shows the levels of wet plasma pNFH (second platform) at various testing points over time;

FIG. 17B shows the levels of plasma or serum BNDF (second platform) at various testing points over time or TBI severity compared to controls;

FIG. 18A is a ROC curve of GFAP D1 healthy controls versus ER TBI subjects;

FIG. 18B is a graph showing GFAP levels in ER TBI subjects versus healthy controls;

FIG. 19A is a ROC curve showing GFAP at 2 weeks post TBI;

FIG. 19B is a graph showing GFAP in ER TBI subjects at two weeks post TBI versus healthy controls;

FIG. 20A is a ROC curve showing NFL;

FIG. 20B is a graph showing NFL levels in ER TBI subjects at less than 24 hours post TBI versus healthy controls;

FIG. 21A is a ROC curve showing NFL at 2 weeks post TBI;

FIG. 21B is a graph showing NFL levels in ER TBI subjects at 2-3 weeks post TBI versus healthy controls;

FIG. 22 shows the levels of TBCB (wet plasma) at various testing points over time;

FIG. 23 shows the levels of WASF-1 (wet plasma) at various testing points over time;

FIG. 24 shows the levels of WASF3 (wet plasma) at various testing points over time;

FIG. 25 shows the levels of VAMP5 (wet plasma) at various testing points over time;

FIG. 26 shows the levels of CAMKK1 (wet plasma) at various testing points over time;

FIG. 27 shows the levels of Ninjurin-1 (wet plasma) at various testing points over time;

FIG. 28 shows the levels of IMPA1 (wet plasma) at various testing points over time;

FIG. 29 shows the levels of ZBTB16 (wet plasma) at various testing points over time;

FIG. 30 shows the levels of PRDX6 (wet plasma) at various testing points over time;

FIG. 31 shows the levels of NFATC1 (wet plasma) at various testing points over time;

FIG. 32 shows the levels of NAA10 (wet plasma) at various testing points over time;

FIG. 33 shows the levels of ING1 (wet plasma) at various testing points over time;

FIG. 34 shows the CSF levels of MOG in multimorbid neurological condition patients having higher levels than those of its pooled healthy control counterpart.

FIG. 35 shows the serum levels of MOG in multimorbid neurological condition patients having higher levels than those of its pooled healthy control counterpart. (p=0.007, two tailed ANOVA analysis);

FIG. 36 shows the CSF levels of MOG in secondary primary multiple sclerosis (SPMS) patients having higher levels than those of its pooled healthy control counterpart. (p=0.033, two tailed T-test analysis);

FIG. 37 shows the serum levels of MBP in multimorbid neurological condition patients having higher levels than those of its pooled healthy control counterpart. (p=0.0285, two tailed T-test analysis);

FIG. 38 is a graph showing temporal profile of serum MOG levels over time in out-of-hospital cardiac arrest (OHC) for four subjects. Two subjects with poor outcome (cerebral performance category/CPC) score of 4) have higher temporal levels of serum MOG as compared to those of two PHCA patients with low (or normal) CPC score;

FIG. 39 is a graph showing temporal profile of serum MBP levels over time in out-of-hospital cardiac arrest (OHC) for four subjects. Two subjects with poor outcome (cerebral performance category/CPC) score of 4) have higher temporal levels of serum MOG as compared to those of two PHCA patients with low (or normal) CPC score;

FIG. 40A is a graph showing various levels of TBI temporal biomarkers over time post TBI; FIG. 40B shows an example of “Gryphon Temporagram” output with severity/grade (high, medium, low) versus time;

FIG. 41A is a graph showing the levels of temporal brain biomarkers profile (Gryphon Temporagram) indicative of a high probability or risk for post-TBI complication, readmission, or persistent PCS;

FIG. 41B is a graph showing the levels of temporal brain biomarkers profile (Gryphon Temporagram) indicative of a moderate probability or risk for post-TBI complication, readmission, or persistent PCS;

FIG. 41C is a graph showing the levels of temporal brain biomarkers profile (Gryphon Temporagram) indicative of a low probability or risk for post-TBI complication, readmission, or persistent PCS;

FIG. 42 is a flow diagram showing the benefits of embodiments of the present invention and how it leads to improved patient care in relation to brain injury;

FIG. 43A is a graph showing Dried Plasma Spot (DPS) samples of TBI patients at Day 1 of injury measuring Glial fibrillary acidic protein (GFAP);

FIG. 43B is a graph showing Dried Plasma Spot (DPS) samples of TBI patients at Day 1 of injury measuring Neurofilament light (NfL);

FIG. 43C is a graph showing Dried Plasma Spot (DPS) samples of TBI patients at Day 1 of injury measuring Total Tau;

FIG. 43D is a graph showing Dried Plasma Spot (DPS) samples of TBI patients at Day 1 of injury measuring phosphorylated threonine 181 Tau (pTau181);

FIG. 43E is a graph showing plasma samples of TBI patient at Day 1 of injury measuring Glial fibrillary acidic protein (GFAP);

FIG. 43F is a graph showing plasma samples of TBI patient at Day 1 of injury measuring Neurofilament light (NfL);

FIG. 43G is a graph showing plasma samples of TBI patient at Day 1 of injury measuring Total Tau;

FIG. 43H is a graph showing plasma samples of TBI patient at Day 1 of injury measuring phosphorylated threonine 181 Tau (pTau181);

FIG. 43I is a graph showing saliva samples of TBI patient at Day 1 of injury measuring Glial fibrillary acidic protein (GFAP);

FIG. 43J is a graph showing saliva samples of TBI patient at Day 1 of injury measuring Neurofilament light (NfL);

FIG. 43K is a graph showing saliva samples of TBI patient at Day 1 of injury measuring Total Tau;

FIG. 43L is a graph showing saliva samples of TBI patient at Day 1 of injury measuring phosphorylated threonine 181 Tau (pTau181);

FIG. 43M is a graph showing longitudinal plasma samples from unique TBI patients collected at 6 months and 12 months for measurement;

FIG. 43N is a graph showing longitudinal saliva samples from unique TBI patients collected at 6 months and 12 months for measurement;

FIG. 44A is a matrix correlation plot of dried plasma spot (DPS) sampling devices, plasma, and saliva for GFAP from data in FIGS. 43A-43N;

FIG. 44B is a matrix correlation plot of dried plasma spot (DPS) sampling devices, plasma, and saliva for NfL from data in FIGS. 43A-43N;

FIG. 44C is a matrix correlation plot of dried plasma spot (DPS) sampling devices, plasma, and saliva for Total Tau from data in FIGS. 43A-43N;

FIG. 44D is a matrix correlation plot of dried plasma spot (DPS) sampling devices, plasma, and saliva for pTau181 from data in FIGS. 43A-43N; and

FIG. 44E shows a 1:1 comparison of DPS sampling recovery of single patient sampling compared to plasma and saliva.

### DESCRIPTION OF THE INVENTION

The present invention has utility in the diagnosis, care, and management of brain specific abnormal neurological conditions in general, and in particular, to traumatic brain injury (TBI) and (TBI-induced) Alzheimer's disease (AD) and Alexander disease, in which a GFAP mutation is implicated in white matter deterioration. The subject invention also has utility in detecting brain specific abnormal neurological condition through detection of temporal biomarkers in biofluid samples (saliva, wet and dried blood, plasma, and serum) from saliva or finger-prick blood collection, including proteins, metabolites, lipids, mRNAs, DNA, cells, microRNAs, and/or autoantibodies thereto. Abnormal neurological conditions can result from neurological trauma that illustratively results from percussive, blast or impact injuries or those resulting from ischemia, or disease. With resort to a microfluidics device or dried sample assays, a detection system is provided that is amenable to usage in the field, at home, and/or during patient transport as well as in the hospital.

The present invention provides early detection and monitoring of subclinical evidence of disease activity (sEDA), before, during, and/or after clinical evidence of disease activity (EDA) occurs.

The ability to use a saliva sample or blood sample drawn from capillaries, as compared to a venous or arterial source affords ease of collection advantages that are attractive for field-, hospital-, and home-based environments. The difficulties associated with capillary blood draw samples include small sample volumes and correlation of detection results with venous or arterial draw blood samples. The usage of dried blood, dried plasma, and/or dried serum spots as a sample facilitates transport and storage, but adds complexity to testing as biomarker recovery, solubility, protection, and degradation must be considered along with the correlation of concentrations of any biomarker detected in biofluid to concentrations of the biomarker in a venous or arterial (tube) blood.

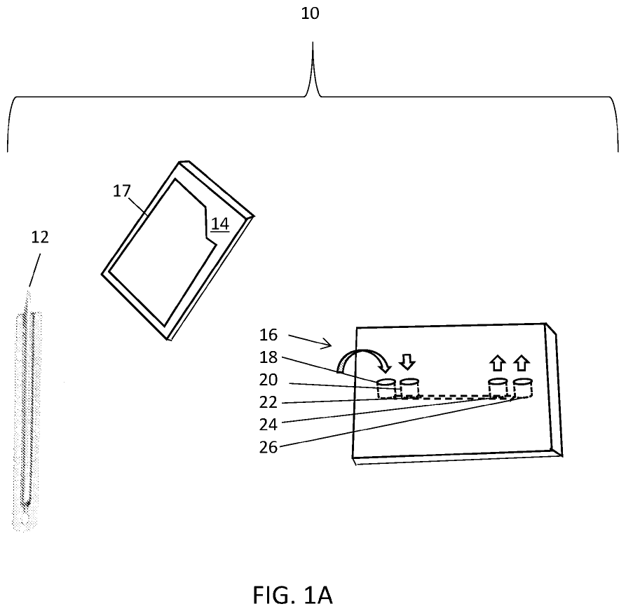

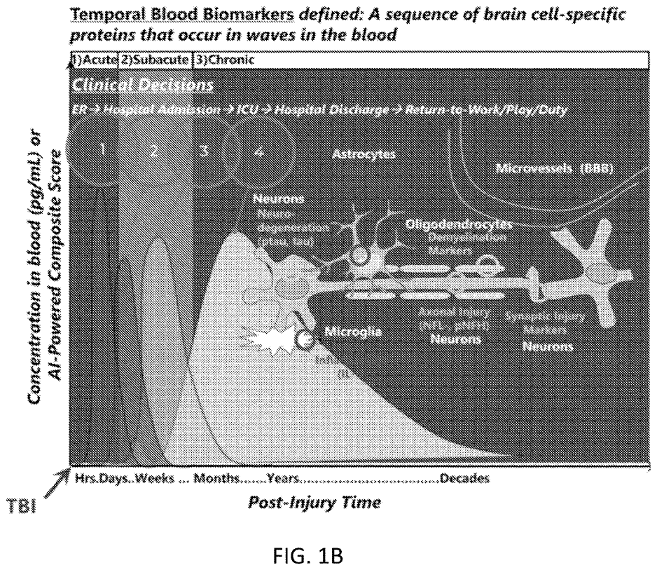

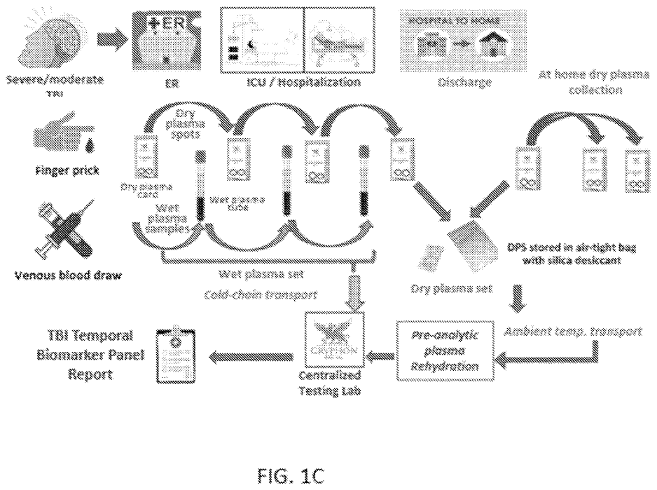

FIG. 1A depicts a kit, shown generally at 10 for a capillary blood biofluid draw. The kit 10 includes a lancet 12 for penetrating the skin to obtain a capillary blood biofluid draw. While capillary blood draws are routinely performed on a subject finger, it is appreciated that heels, forearms, earlobes are illustrative of other conventional sites for obtaining a capillary blood draw. An expressed drop of blood has a typical volume of 20 to 50 microliters. The resulting drop of blood is transferred directly or with resort to an applicator (not shown) to a porous substrate 14 or a microfluidic device 16. The porous substrate 14 in some inventive embodiments has known blood retention volume such that a unit area of the porous substrate 16 correlates to a known volume of blood. Typically, a square millimeter of porous substrate 16 retains between 1 and 20 microliters of the capillary blood, with the thickness and wickability of the substrate toward blood being significant factors in blood retention volume of the porous substrate 16. After absorption, the liquid component of the blood drop evaporates leaving the biomarkers and corpuscular components from the blood drop retained in the porous substrate. In some inventive embodiments, a surface coating 17 is present on the porous substrate14 with channels therein sized to permit blood plasma constituents to pass therethrough while cellular blood components are preferentially excluded from entering the porous substrate 14.

Alternatively, or in concert with the porous substrate 14, a microfluidic device 16 is provided in the kit 10. The microfluidic device 16 has an inlet 18 for receipt of a drop of blood as denoted by the curved inlet arrow thereto. A buffer inlet 20 is also provide to dilute the blood and convey the components thereof, including the biomarkers of interest through a channel system, shown in simplified form at 20. One or more outlets 22 and 24 are provided for exhausting waste, fractions containing biomarkers, of the like. It is appreciated that determination of the amount of a given biomarker in a sample of biofluid provides clinically useful information about the nature of an abnormal neurological condition, still other information useful in treatment may be present in the detection of isoforms, the degree of splicing, phosphorylation, other chemical and post-translational modifications, mutations, or a combination thereof for in a given biomarker. Tau protein is exemplary of a biomarker that includes secondary information such as phosphorylation at specific amino acid residues. Elution of biomarkers from the microfluidic device 16 affords the option to further evaluate biomarkers through techniques not incorporated to the microfluidic device.

In some inventive embodiments, a device is provided in the kit 10 that is a lightweight, non-refrigerated, scalable, cost-effective, minimally invasive dried plasma spot (DPS) sampling device. Such a device, as shown for example with reference to 14 or 16 is designed specifically for self or caregiver-assisted finger-prick capillary blood biofluid collection at home or in the field (e.g., a sporting event or austere military environment) or hospital. The ability for individuals with no training or limited training to collect a capillary blood biofluid sample contemporaneously with a possible TBI, TBI induced Alzheimer's disease (AD) or other event indicative of a negative change in neurological condition enhances the likelihood of detecting one or more biomarkers that have a peak concentration in blood within 4 hours of injury thereby providing important clinical information as to the types of cells implicated in the event. Alexander disease is also noted to detectable according to the present invention through GFAP protein sequencing from the sample. This is depicted in FIG. 1B where the relative kinetics of various biomarkers are shown along with cell types that once damaged are the source of particular biomarkers. The present invention by eliminating the need for venipuncture collection also eliminates the need for refrigerated transport and storage of tubes of blood specimens associated therewith. Dried (whole) blood spot sampling as part of prenatal testing and reports of longitudinal studies for non-CNS indications is well known (Curtis, Ambrose et al. 2014, Mussa, Ciuffreda et al. 2019). However, is previously not been applied to abnormal neurological condition biomarker detection-particularly with respect to capillary blood biofluid collected as dried plasma or dried serum or a saliva biofluid sample.

The present invention provides sensitivity immunoassays with low sample volume requirements: a temporal biomarker panel for capillary blood samples or saliva samples for biomarkers with either comparatively high concentrations or for which sensitive detection techniques exist. Biomarkers operative in the present invention using biofluid illustratively include: P-Tau (181, 202, 217, 231, 396/404), Tau, IL-6, IL-15, GFAP and breakdown products (BDPS) thereof, NF-L, MOG, aquaporin 4, apolipoprotein E4, SAA, adenosine, myoinositol, norepinephrine, NAA, NAAG, glutamate, glutamine, combinations thereof, a metabolic breakdown products of any of the aforementioned. It is appreciated that the breakdown products of GFAP and the temporal cascade thereof are well known to the art.

In some inventive embodiments, inventive capillary blood biofluid draw is used in combination with conventional collection of venous or arterial blood samples for testing to establish the analytical comparability of an inventive sampling panel of temporal (acute, subacute, and chronic) CNS and non-CNS blood biomarkers (FIG. 1B) with samples from both capillary blood biofluid collection (e.g., finger-prick capillary blood collection with dried plasma spot (DPS) sampling) and from venous blood collection. Some notable differences between the conventional venous or arterial blood samples and those of DPS include at least one of differential relative levels of recovery for a given biomarker, composite scores, and temporal profile thereof. Therefore, differences cannot be predicted from the amino acid sequence or structure of a given biomarker.

According to the present invention, biofluid is analyzed for a given biomarker from a saliva sample or dried blood spot or with a microfluidics device. Exemplary of a microfluidics device operative herein is that disclosed in US20090053732 A1.

As used herein, the abbreviations “D”, “mo.” And “Y” are used synonymously with day, month, and year, respectively.

As used herein, “clinical EDA” illustratively includes subject imaging, CSF biomarker, cognitive assessments, and other clinical measures not based on blood biomarkers.

It is to be understood that in instances where a range of values are provided that the range is intended to encompass not only the end point values of the range but also intermediate values of the range as explicitly being included within the range and varying by the last significant figure of the range. By way of example, a recited range of from 1 to 4 is intended to include 1-2, 1-3, 2-4, 3-4, and 1-4.

According to embodiments, a lightweight, non-refrigerated, scalable, cost-effective, minimally invasive biofluid sampling device, such as a Dried Plasma Spot (DPS) sampling device, is provided that is designed specifically for medic-assisted blood collection in the field, as part of a single platform solution (SPS) to substantially improve the feasibility of sample collection for TBI patients prolonged field care (PFC), during transport from the field to a hospital, during their hospital stay, and during their time as out-patients away from a hospital setting. By maintaining a single sampling platform throughout patient management and disposition, a composite score (threshold) is calculated without the need for difficult, to near impossible, bridging studies between point-of-care measurement solutions such as the iSTAT platform and more sensitive, hospital-based solutions such as the core lab Anility immunoassay platform. Moreover, unlike the iSTAT platform, DPS sampling eliminates the need for venipuncture collection or refrigerated transport and storage of blood specimens.

Embodiments of the present invention utilize DPS tests for biofluid TBI biomarkers. According to some inventive embodiments, 5-25 μL of plasma are placed onto a DPS from 3-4 drops (70 μL) of finger prick capillary blood. According to embodiments, the present invention includes a dried plasma spot (DPS) sampling device. According to embodiments, the present invention uses a five-step sequence from serial sampling of 70 μL (3-4 drops) of finger-prick blood for capillary transport of preselected amount (e.g. 10 μL) of plasma to the DPS collection disk. The system collects 2×10 μl plasma from a finger stick of blood in the range of 35-55% hematocrit. By exploiting the biomarkers and techniques of WO2016209147A1 and WO2020050770A, all functionalities of the system including, as shown in FIG. 1B, blood pre-metering (1), plasma extraction (2), plasma metering (3) and collection into a dried sample format (4), are passively driven. The design allows for manipulating liquids only through capillary forces enabling a completely autonomous multifunctional system. The system is constructed in some inventive embodiments using foil-based microfluidic technologies enabling high throughput manufacturing by roll-to-roll. This device is based on a successful dried blood spot sampling device that is capable of measuring analytes with unprecedented volumetric precision from varying applied sample volumes and hematocrit levels.

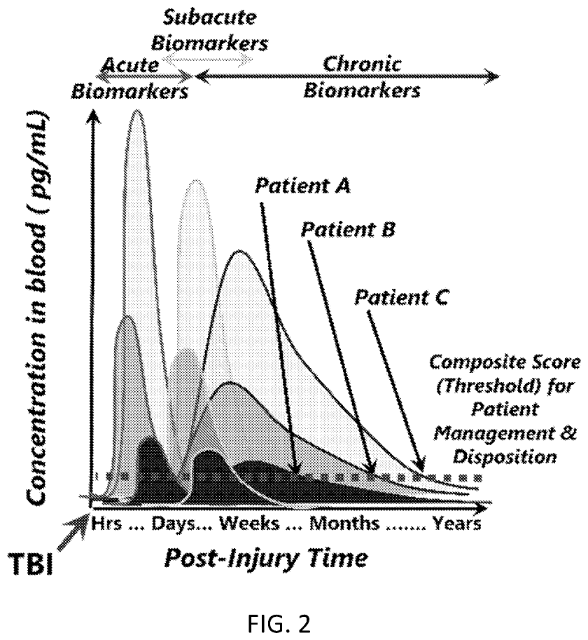

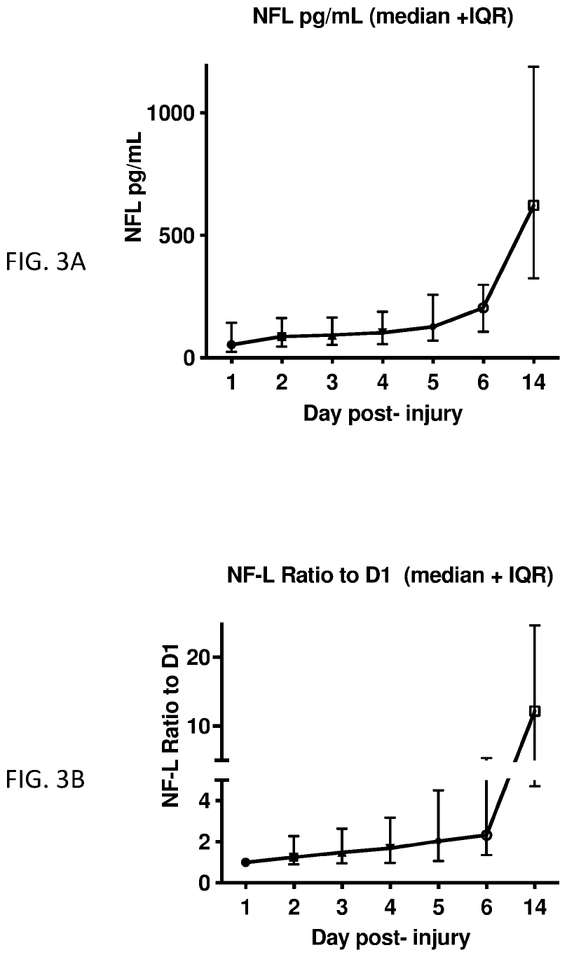

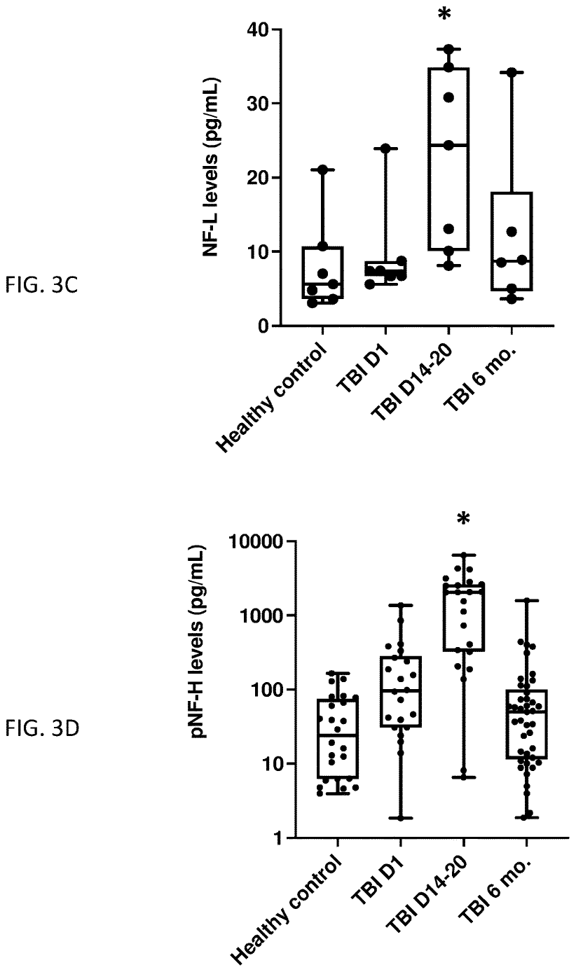

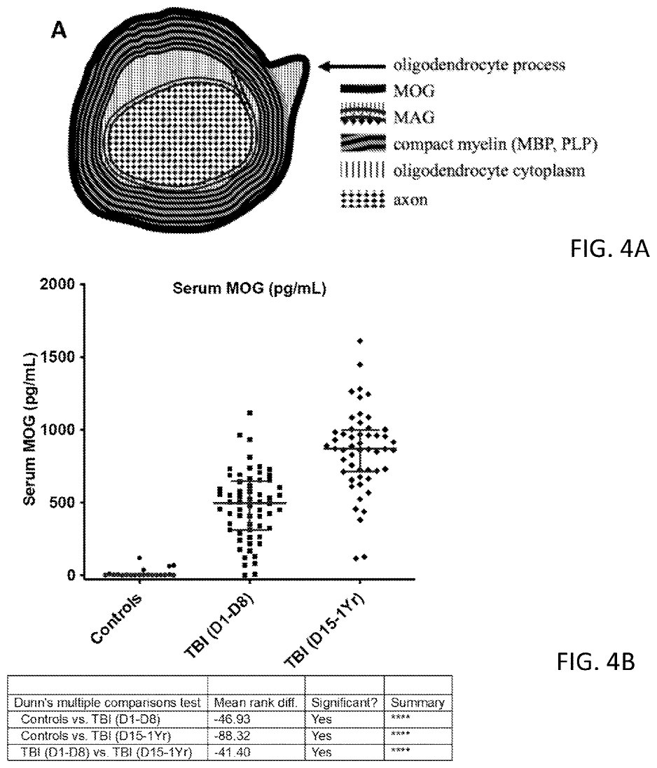

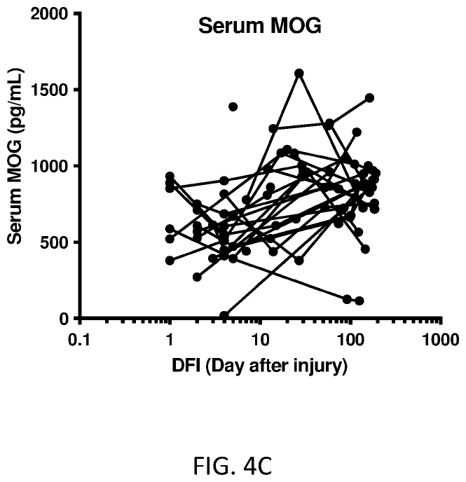

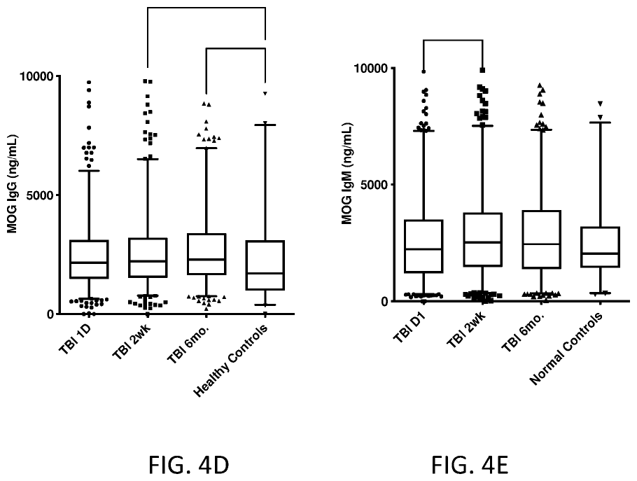

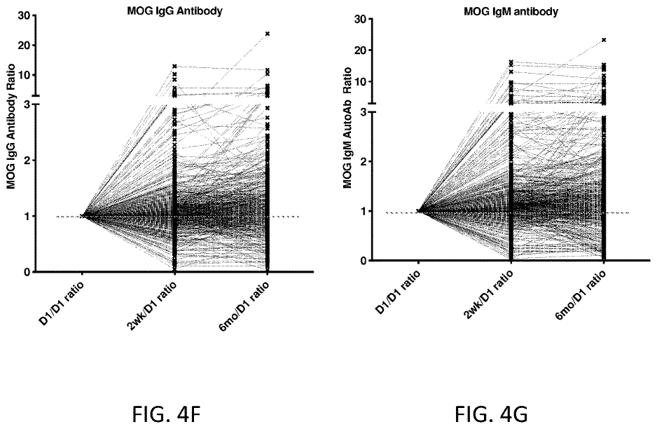

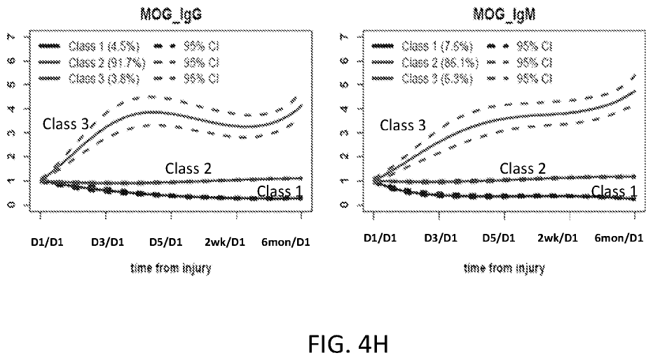

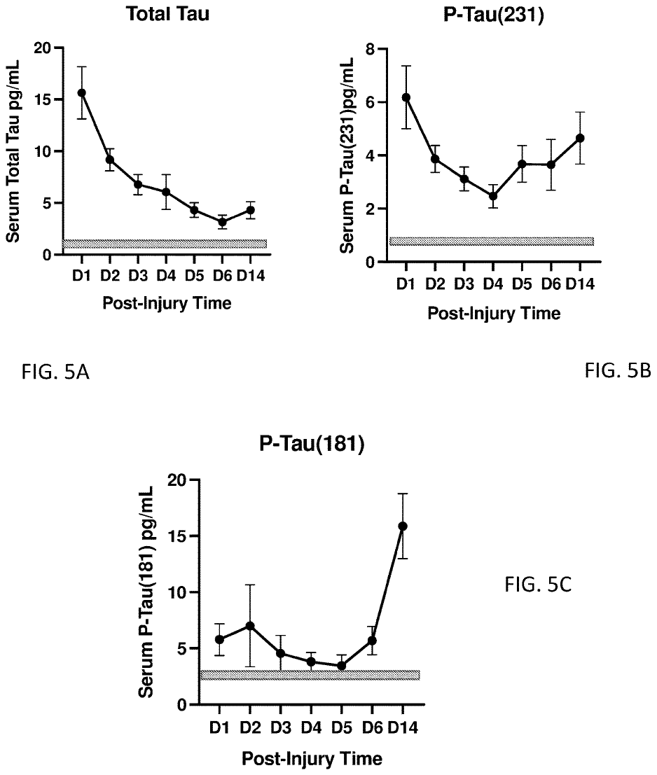

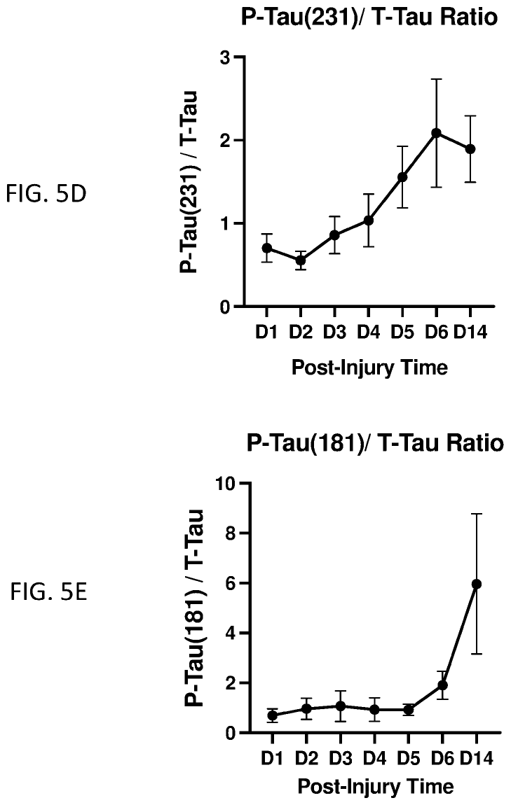

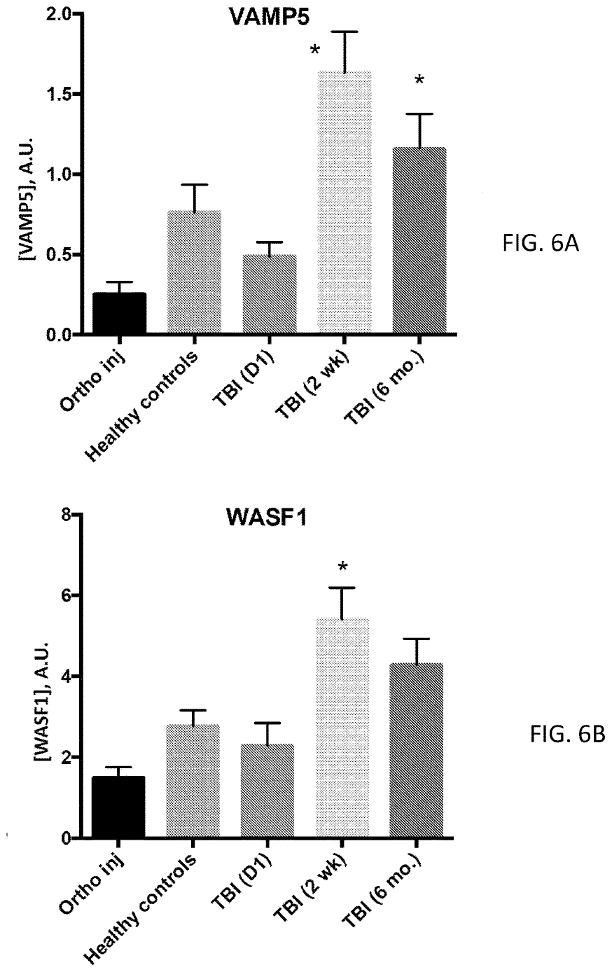

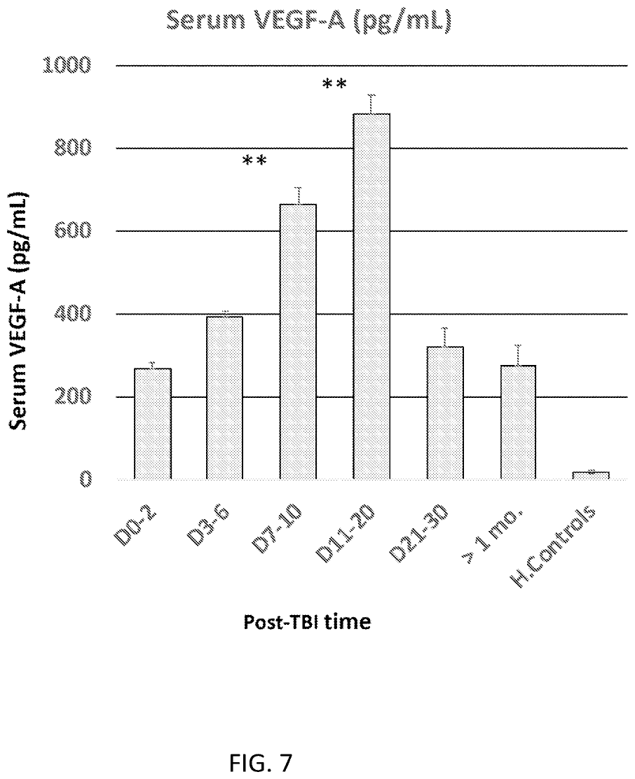

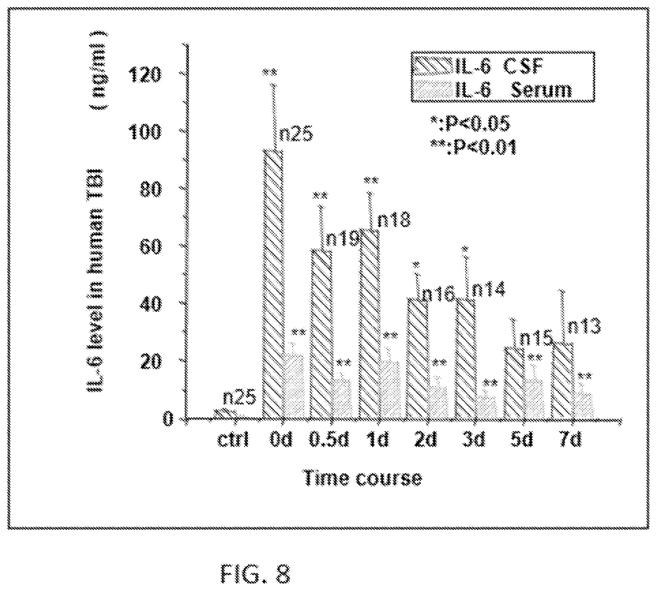

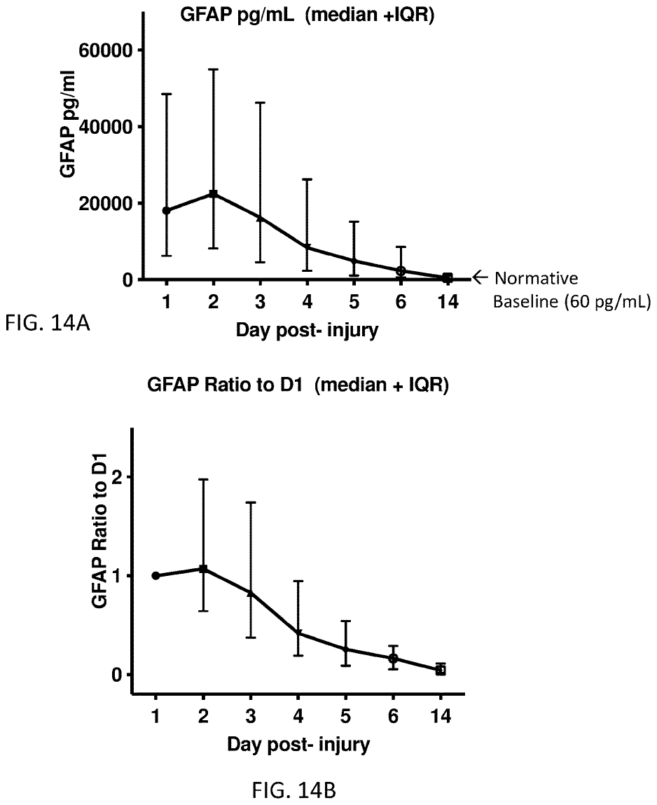

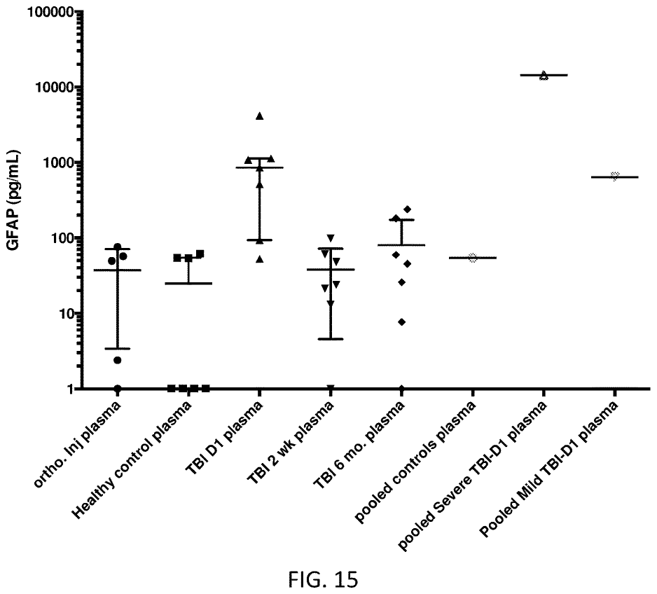

The present invention provides a fast and effective single platform solution (SPS) for clinical validation of a single panel of temporal brain milieu biomarkers with distinct and complementary biomarkers representing vulnerable brain cell types, subcellular structures, TBI-associated pathophysiologic events and/or subphenotype, including axonal injury (neurofilament proteins NF-L, pNF-H) (FIGS. 3A, 3B and 3C) myelin damage/white matter injury (myelin oligodendrocyte glycoprotein/MOG) (FIGS. 4A, 4B, 4C), MOG antibody IgG and IgM (FIGS. 4D, 4E, 4G and 4H), neurodegeneration (Tau, p-Tau (T231) (FIGS. 5A, 5B, 5C, 5D, 5F), synaptic injury (Vesicle-associated membrane protein 5 (VAMP5), Wiskott-Aldrich syndrome protein family member 1 (WASF1) (FIGS. 6A and 6B), vascular injury/remodeling marker VEGF-A (FIG. 7), neuroinflammation marker (IL-6) (FIG. 8) and complemented with astroglia injury marker GFAP (FIGS. 14A, 14B, and 15) which is by far the most robust marker in TBI diagnosis (Czeiter, Amrein et al. 2020). The inventive SPS combines high frequency DPS sampling with high sensitivity immunoassays of a unique panel of blood based temporal TBI biomarkers (acute, subacute, and chronic), as shown in FIG. 2, to monitor patients' progression and phenotype individual trajectories to reconstruct and inform on key decisions from the initial injury in the field to hospital and home settings. FIG. 2 is a graph showing high frequency dried plasma spot (DPS) sampling and high sensitivity immunoassays of a proprietary panel of blood based temporal TBI biomarkers as a single platform solution (SPS). Key decisions include triage decisions, acuity of injury, higher level care needs, management and disposition, and return-to-duty/work/play decisions (Orszag and Emanuel 2010, Brito, Costantini et al. 2019, Shrank, DeParle et al. 2021).

According to some inventive embodiments, a single high sensitivity immunoassay platform is provided with multiplexing capabilities for testing a panel of up to 7 biomarkers indicative of TBI and in other embodiments up to 20 such biomarkers. According to other inventive embodiments, the inventive immunoassay platform utilizes the First platform described above. The inventive diagnostic is carefully designed to include several key TBI-tracking protein biomarkers with distinct and complementary acute and post-acute temporal profiles to identify the development of secondary injuries, PCS, or immune responses—factors which inform return-to-duty/work/play decisions.

According to some inventive embodiments, the inventive CP described above facilitates repeated finger-prick-based blood sampling from the same subjects with a single platform during the full disease course of TBI. For example, the CP is ideal for collecting prehospital specimens including during in field care, transport, at a hospital or other care facility, and after discharge from the hospital. Advantageously, the present invention reduces biosampling burden.

The UniProt Reference numbers for the biomarkers used in embodiments of the present invention are as follows: NFL—Neurofilament L (UniProtKB—P07196, P07197, P12036 (NFL_HUMAN)) GFAP—Glial fibrillary acidic protein (astrocyte health) (UniProtKB—P14136 (GFAP_HUMAN)) GFAP breakdown product (BDPS) 38K, Tau/p-Tau per WO2020124013A1, TCBB—Tubular folding cofactor B (elevated with injury) (UniProtKB—Q99426 (TBCB_HUMAN)) Non-brain specific subacute markers IMPA1—Inositol Monophosphatase 1 (UniProtKB—P29218 (IMPA1_HUMAN)) NNA10—N-terminal acetyltransferase 10 (UniProtKB—P29218 (IMPA1_HUMAN)) BDNF—Brain Derived Neurotrophic Factor (UniProtKB—P23560 (BDNF_HUMAN)) WASF1—Wiskott-Aldrich syndrome protein family member 1 (UniProtKB—Q92558 (WASF1_HUMAN)) WASF3—Wiskott-Aldrich syndrome protein family member 3 (UniProtKB—Q9UPY6 (WASF3_HUMAN)) CAMKK1—Calcium/Calmodulin Dependent Protein Kinase Kinase 1 ((UniProtKB—Q8N5S9 (KKCC1_HUMAN)) Ninjurin-1 (vascular injury) (UniProtKB—Q92982 (NINJ1_HUMAN)) ICAM1 (Intercellular Adhesion Molecule 1) (UniProtKB—P05362 (ICAM1_HUMAN)).

Acute and delayed axonal injury has been suggested after brain injury but it can be challenging to access. Neselius et al found increased CSF levels of pNF-H following bouts among amateur boxers (Neselius, Zetterberg et al. 2013), and pNF-H also appears to be a predictor of mortality after brain injury in children (Hu, He et al. 2002). In addition, serum NF-L also appears elevated in American football players over the course of a season and in TBI subjects. Importantly, the release of NF protein into biofluids is a delayed process with respect to the initial insult (days following insults) (Yuan and Nixon 2021). More recent data shows that NF-L continues to rise within the first 14 days post injury (dpi) with a clear decline after 1-3 mo (Shahim, Politis et al. 2020). High frequency serum sampling from severe-moderate TBI is used to define both NF-L (third platform) rising from day 1 to day 14, as shown in FIGS. 3A-3B. In addition, with longitudinal, severe-moderate TBI patients' serum samples, the present invention shows that both NF-L and pNF-H in fact peaking at the D14 to Day 20 post-injury time, before receding at 6 mo. post-injury. However, even after 6 mo., NFL and pNF-H levels are still higher than in their respective normal control counterparts.

White matter injury (WMI) is of great clinical importance among brain injury patients. WMI is often associated with myelin injury and demyelination. As noted above, oligodendrocytes form myelin-sheaths which protect the long axons in the brain in the white matter (myelinated fiber tracks), as shown in FIG. 4A. However, it has been challenging to assess WMI and myelin injury and demyelination non-invasively. FIGS. 4B-4C show pilot data on the demyelination marker MOG showing sustained elevation of levels in the subacute to chronic phases of TBI. Specifically, FIG. 4A shows localization of MOG at the external lamellae of myelin sheaths. While not intending to be bound to a particular theory, it is believed that due to its outside surface location on the myelin sheath, MOG is highly vulnerable to structural damage to myelin leading to is release to circulation FIG. 4B shows serum MOG elevations in acute (D1-8) and subacute-chronic (D15-1 yr) TBI samples. Median comparisons (Kruskal-Wallis test, ***P<0.001 compared to control, p<0.01. FIG. 4C shows Chronic time course of serum MOG showing the temporal profile for 12 patients. The myelin sheath found in CNS serves as an insulator to increase the velocity of axonal impulse conduction. MOG is found on the external lamellae associated with the myelin sheath. Myelin basic protein (MBP) is found in the compact myelin layer of the myelin sheaths. In the past, MOG, has only been studied as possible biomarker for demyelination diseases such as multiple sclerosis (MS)(Galazka, Mycko et al. 2018) but not for TBI or Alexander disease. However, the present invention is based on the finding that in a human MOG ELISA assay there is a robust release of MOG into human serum from TBI patients from the acute (1-10 dpi) to chronic (10 dpi to 12 mo.) phases, as shown in FIG. 4B, as well as Alexander disease subjects. Importantly, MOG biomarker presents with chronic phase levels higher than acute levels in 14 out of 15 individual TBI patients, as shown in FIG. 4C, suggesting demyelination continues long after initial injury. This set of data and observation regarding blood-based measurement of MOG as a delayed demyelination/WMI biomarker following acute brain injury such as TBI is novel and has never described before in publications or patent filing to our knowledge. MOG sustained elevations and its temporal profile of higher levels in the chronic phase post-injury (e.g. 1 mo. to 1 yr) as compared to acute phase is unique and unobvious among other brain injury biomarkers. Importantly, the present invention demonstrates that this delayed and continuing release of MOG implicates a previously unknown continuing damage to the myelin sheath and possible chronic vulnerability of its associated white matter. Furthermore, MOG is a recognized autoantigen in certain central nervous system autoimmune diseases. MOG antibody disease (MOGAD) is defined as neurological, immune-mediated disorder in which there is inflammation in the optic nerve, spinal cord and/or brain. MOG antibody (autoantibody) production in the immune system can in fact leading to CNS demyelination and Neuromyelitis optica (NMO) (Marignier et al., Lancet Neurol. 2021, vol 20, 767). Previously, it was shown that another brain protein, glial fibrillary acidic protein (GFAP) could invoke autoimmunity response following TBI (Zhang Z, et al. PLoS One. 2014, vol. 9(3):e92698; Wang, K K W, J. Neurotrauma, 2016 vol. 33:1270). But due to its sustained release, MOG is posited to create a high risk to TBI patients for developing autoantibody response against MOG following the initial brain injury. Since MOG antibody disease is already documented, the present invention exploits high MOG levels as a diagnostic test result to predict a patient's risk for developing MOGAD or MOGAD-like autoimmune disorder. The metabolites of MOG are known (Peschl, Patrick et al.).

As shown in FIG. 4D, both mean anti-MOG antibody IgG and IgM isoforms at 2 week and 6 mo. post TBI patients (all severity spectrum, N=500) are significantly elevated as compared to those of healthy controls (N=150), while FIG. 5E shows that post-TBI 2 week mean anti-MOG antibody IgM levels are higher than their mean TBI D1 levels. The delayed increases of MOG antibodies is consistent with the autoimmunity response. FIG. 4F and FIG. 4G further show, when the same subject MOG IgG and IgM levels were expressed as a ratio to their D1 IgG and IgM levels, respectively, it is clear that a subset of TBI patients have elevated MOG antibody by 2-fold to more than 20-fold. (as IgG or IgM) at 2 week and 6 month post injury. FIG. 4 H shows that machine learning based trajectory analysis in fact can categorize MOG antibody IgG and IgM levels into three hidden trajectory classes: namely class 1 declined trajectory, class 2 flat trajectory, and class 3 high or increased trajectory using day 1-5, 2 week and 6 mo. post-TBI samples from the same subjects. Of note is that there are 3.8% and 6.3 patients who developed elevated MOG IgG and MOG IgM over time, Without intending to be bound to a particular theory, it is believed that such patients might be associated with high sustained levels of blood levels of MOG over post-brain injury time points, and thus, the present invention affords utility in measuring MOG biomarkers in blood (or serum or plasma) to inform clinicians and the patients the risk of developing MOGAD or MOGAD-like disorders.

As noted above, moderate to severe TBI has been shown in autopsy studies to result in increased amyloid and microtubule associated protein tau (Tau) deposition in the brain. Elevated levels P-Tau are seen in the brain in chronic traumatic encephalopathy for years following mild TBI or concussion (McKee, Cantu et al. 2009, Omalu, Hamilton et al. 2010). Previous innovations provide an ultra-high sensitivity surrounded optic fiber immunosorbent assay with rolling cycle amplification (RCA-SOFIA) for total tau (T-tau) and P-Tau. Such platform's sensitivity (fg/mL) is even beyond the next most sensitive platform—third. Accordingly, T-Tau is readily detected in serum/plasma, the much less abundant P-Tau (231) is also detectable in blood. It has been surprisingly found with the RCA-SOFIA assays that P-Tau (231) or P-Tau (217) and total Tau have a delayed rise adays after TBI. FIG. 5A is a scatter plot of median/IQR for serum P-Tau pilot data temporal profile. FIGS. 5B and 5C are scatter plots of median/IQR showing distinct serum P-Tau (231) and P-Tau (181) temporal profile. Similar results exist for P-Tau (217). In addition, FIGS. 5A-5C, third T-Tau and P-Tau (Thr-231) assays can differentiate serum samples from severe TBI from control. In these figures, TBI (N=45 each) are distinguishable from control (n=30). T-Tau and two P-Tau (231,181) also have distinct temporal profile, notably both P-Tau have a U-shape curve and a second peak at D14 post-TBI Both initial (D1) Tau species and 14D are higher than control counterparts. Control: P-Tau (231, 0.876 μg/mL; T-Tau, 0.342 μg/mL; p-Tau (181), 3.61 pg/mL (depicted as blue horizontal bars). Furthermore, FIG. 5D and FIG. 5E, respectively show the ratio of P-Tau (231)/total Tau and P-Tau (181)/Total Tau have very different temporal profile. It is appreciated that the ratio of P-Tau between freely circulating in serum relative to plasma from the same subject provides additional information the condition of the subject.

Synaptic injury and neuronal cell body injury are underappreciated in TBI. Loss of synaptic connectivity could be permanent and have profound consequences on brain function. Vesicle-associated membrane protein 5 (VAMP5) belongs to a VAMP-synaptobrevin family of proteins that also include VAMP2 (synaptobrevin-2), VAMP3 and VAMP7. That are small (18 kDa) integral transmembrane proteins involved with in neurotransmitter-loaded synaptic vesicles docking onto the presynaptic terminal. In parallel, Wiskott-Aldrich syndrome protein family member 1 (WASF1), also called WASP-family verprolin homologous protein 1 (WAVE1) and iandother WASP-family member protein WASF3 are enriched in the brain and localized in the cell body cytoplasma of neurons. WASF1 has been shown to associate with an actin nucleation core Arp2/3 complex while enhancing actin polymerization in vitro. WASF1 is involved in transport of vesicle-bound proteins such as Amyloid precursor protein (APP) to the cell surface. Significant subacute elevations for both VAMP5 and WASF1 are observed with a neurology panel (192 analytes). Specifically, VAMP5 and WASF1 levels were elevated at two weeks past six months post TBI in comparison to 1 dpi TBI and healthy control specimens, as shown in FIGS. 6A and 6B. FIG. 6A shows pilot data on ahigh sensitivity immunoassay platform showing the synaptic marker VAMP5 at elevated levels in TBI patients 2 weeks and 6 months following TBI, as compared to Healthy controls and TBI D1. *p<0.05, ANOVA of means; n=5-7. FIG. 6B shows data on the high sensitivity immunoassay platform showing the synaptic marker vesicular trafficking marker WASF1 at elevated levels in TBI patients 2 weeks and 6 months following TBI, as compared to healthy controls and TBI D1. *p<0.05, ANOVA of means; n=5-7. Levels are shown in arbitrary units (A.U.). This suggests that WASF1 and VAMP5 are markers for delayed synaptic and vesicular transport dysfunction.

Vascular injury-vascular remodeling markers represent another key phenotype and feature of brain injury. For example, the neuro-vasculature (arteries, veins, and microvasculature can be damaged directly or indirectly duration the course of brain injury. Thus, it follows that monitoring the injury or recovery/remodeling at different time points post-injury is clinically important. FIG. 7 shows that vascular endothelial growth factor (VEGF-A) in serum rising above normative control levels even on day 0-2 after TBI, but its levels do not peak until D7-10 and D11-20, suggesting a delayed vascular involvement and remodeling effect. There are other VEGF isoforms, including VEGF-D, VEGF-D AND VEGF-B.

Neuroinflammation markers represent a major systemic response to brain milieu environment changes (Simon, McGeachy et al. 2017). One of the most robust biomarkers is interleukin-6 (IL-6). Post-TBI IL-6 cerebrospinal fluid (CSF) and serum levels show an early rise above baseline control levels, as shown in FIG. 8. FIG. 8 is a graph showing pilot data showing the temporal profile of serum IL-6 elevations in acute and subacute severe TBI with the horizonal line being the median level for normal controls. For most subjects, levels thereof tend to decay over time, however its serum levels stay elevated in at least half of the subjects examined in the pilot study.

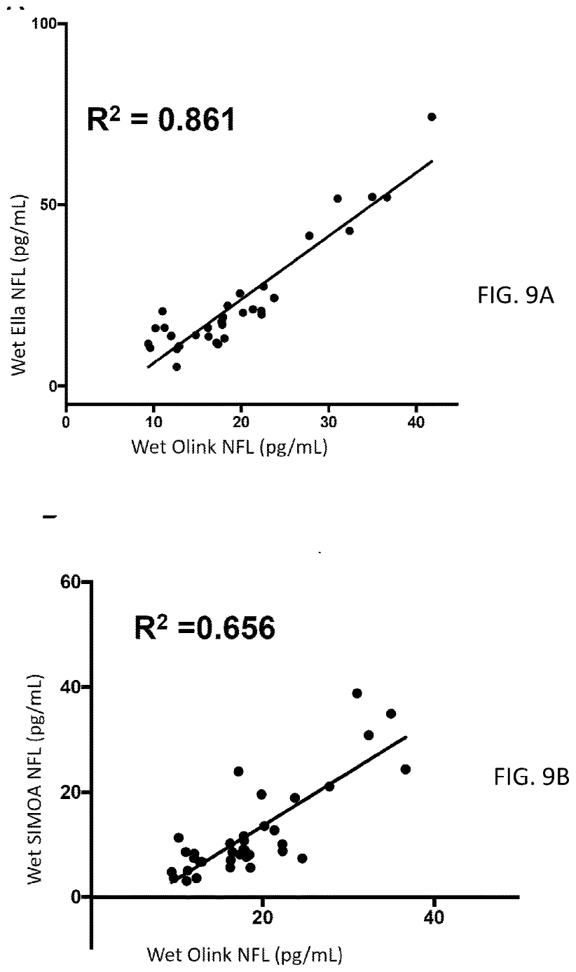

Measurement of brain injury biomarkers with multiple platforms: FIG. 9A and FIG. 9B show that three conventional assay platforms are reporting similar levels of wet plasma levels from TBI patients and controls. This informs the ability of the present invention in using multiple available assay platforms to make such biomarker assessment.

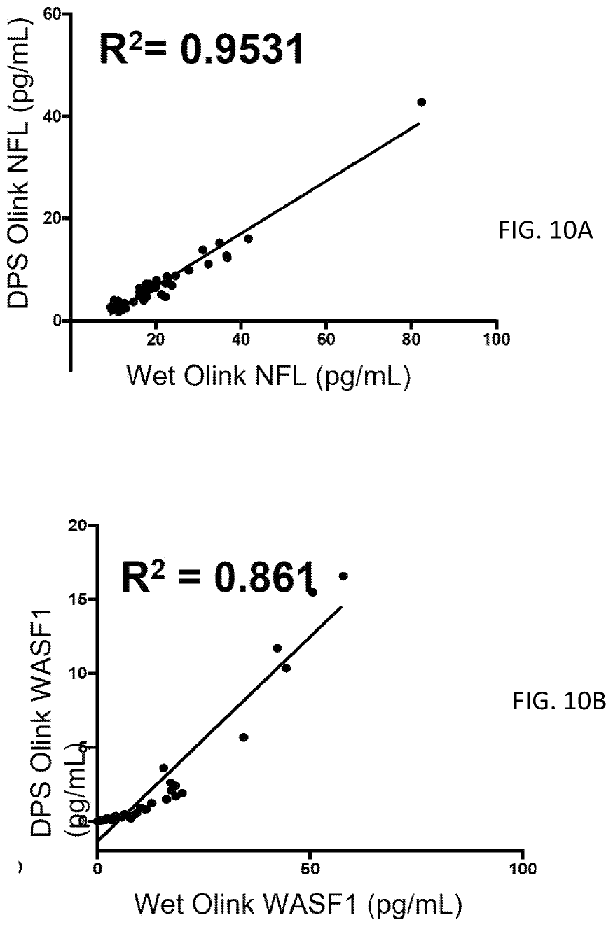

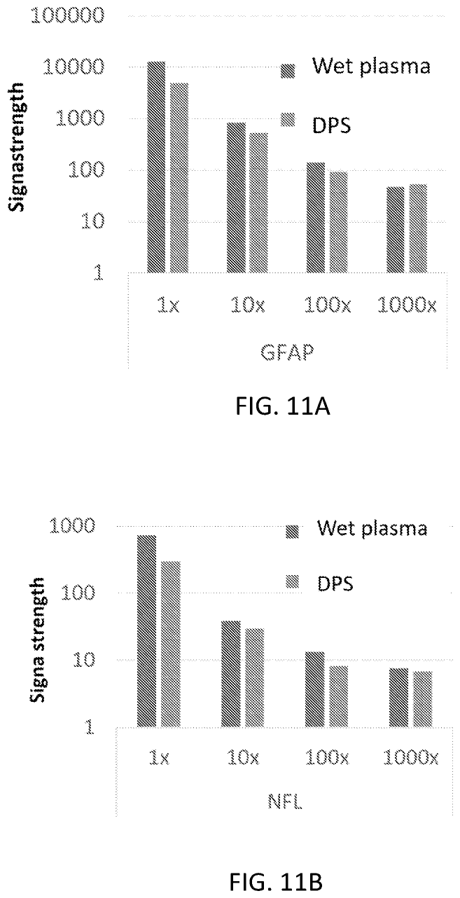

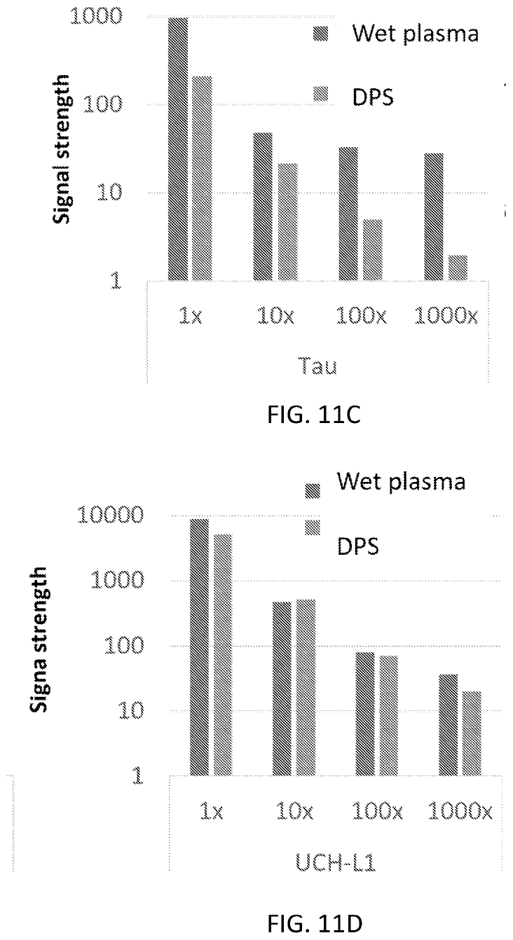

Measurement of brain injury biomarkers with dry plasma spot recovered samples versus wet plasma samples. To increase access of patient care to patients having difficult accessing medical clinical or diagnostic laboratory for blood drawing, it is desirable and of clinical utility to make blood sample collection remotely. In addition, finger prick-based blood drawing and/or saliva sampling is advantageous over blood collection by venous blood draw for its simplicity, minimally invasiveness and that it can be self-administrated or administrated by a caretaker rather than medically trained professional, as shown in FIG. 1. Also the dry plasma samples are stable and can be stored and transported or shipped to analytical site at either ambient temperature. Once arriving at the analytical testing site, a dry plasma rehydration procedure is optionally implemented. Importantly, FIG. 10A and FIG. 10B show that two of our candidate brain injury secondary protein biomarkers, NFL and WASF1 showed strong correlation between rehydrated dry samples and their wet plasma sample counterparts among over 30 TBI plasma samples Again, by spiking into four TBI biomarker analyte standards as a mixture at different dilutions into healthy control wet plasma and its DPS counterparts, FIGS. 11A, 11B, 11C and 11D shows that there are parallel concentration-response dilution curves for all four TBI markers.

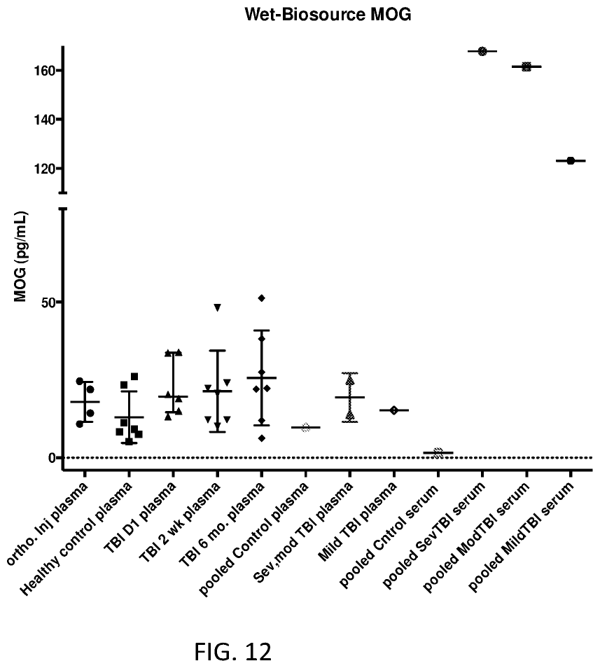

FIG. 12 shows MOG measurement in various blood samples were characterized using sandwich ELISA assay. Importantly, TBI 2 week and 6 mo. plasma have higher MOG levels than their control plasm counterparts by about 2- to 3-fold. However, it is noted that pooled severe, moderate and mild TBI serum samples are up 30- to 50-fold higher than their pooled control serum counterpart. The present invention provides the surprising result that MOG measurement in serum (or rehydrated dry serum samples) can be superior to similar measurement in plasma matrix samples. Without intending to be bound to a particular theory, MOG, due to its two lipid membrane associated regions might be partitioned or bound to fatty acid or phospholipid and other lipid in the plasma and thereby partially escaping detection but is only released into the serum compartment upon blood coagulation.

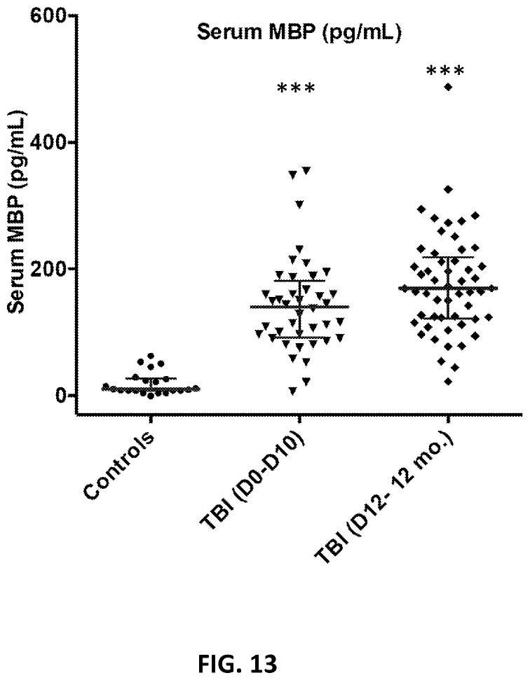

FIG. 13 shows that another myelin/WMI marker is also elevated in TBI patient serum samples collected at D0-D10 as well as from D12 to 1 month. This temporal profile again parallels those for MOG, further confirming that myelin markers have an acute, as well as sustained chronic elevations in blood following brain injury.

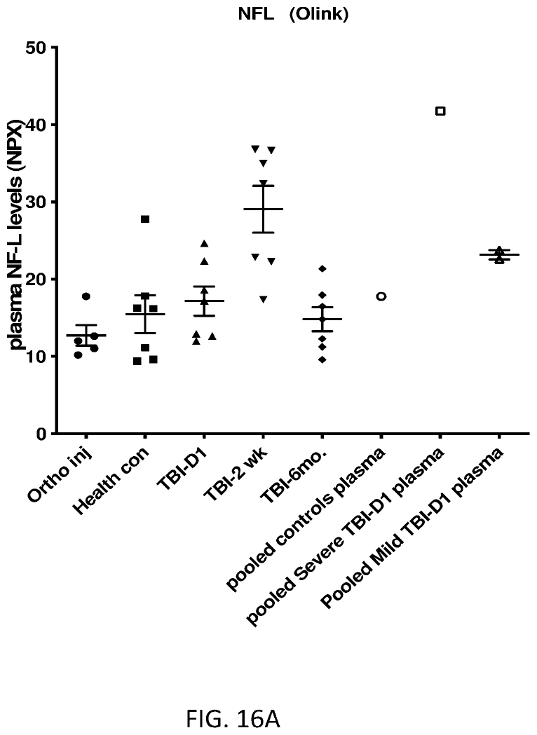

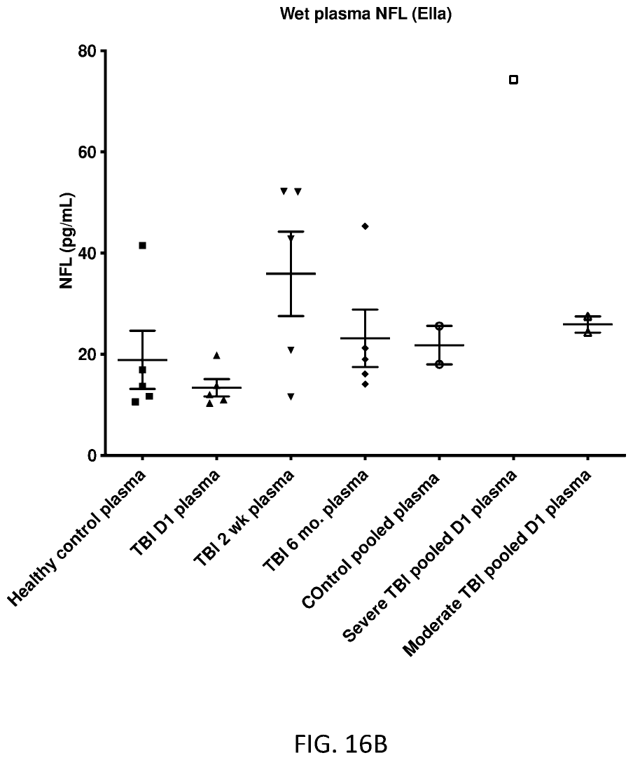

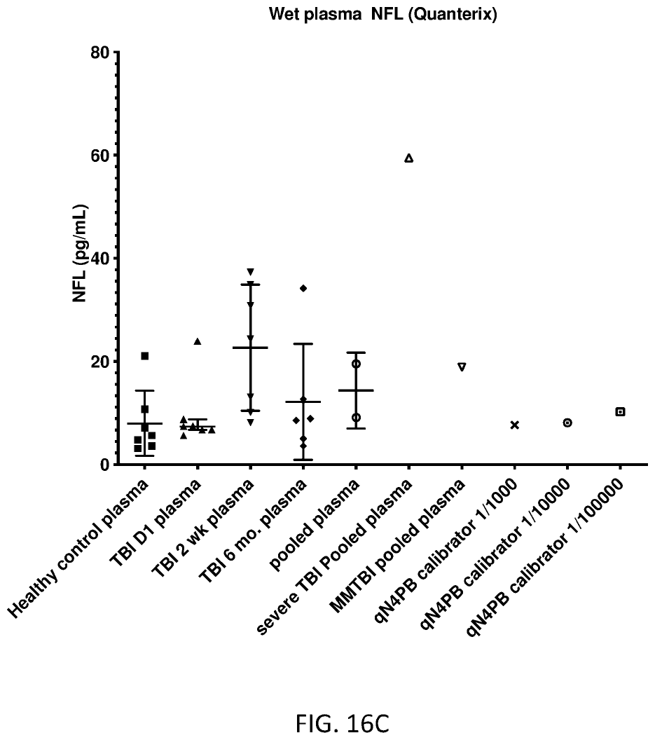

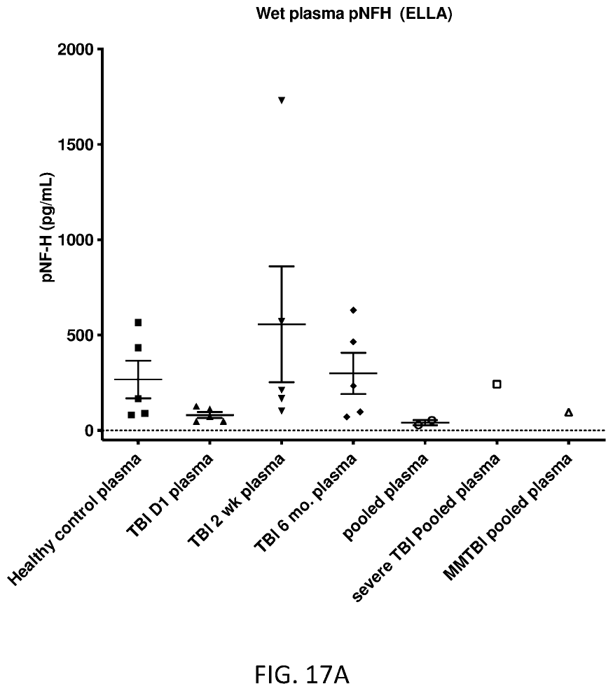

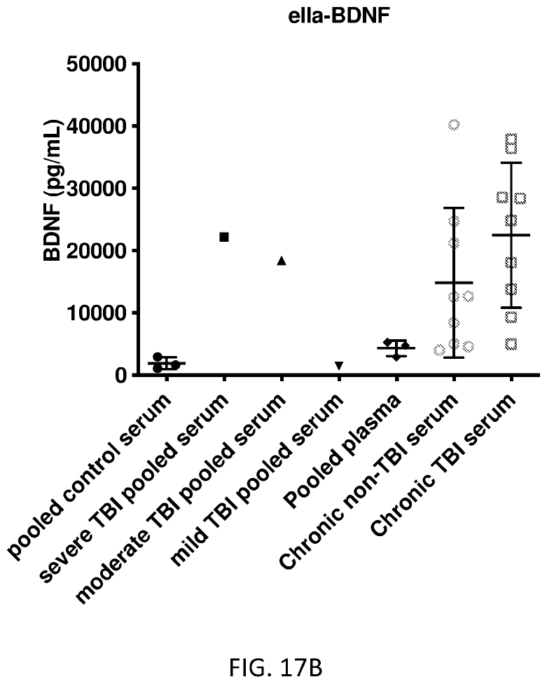

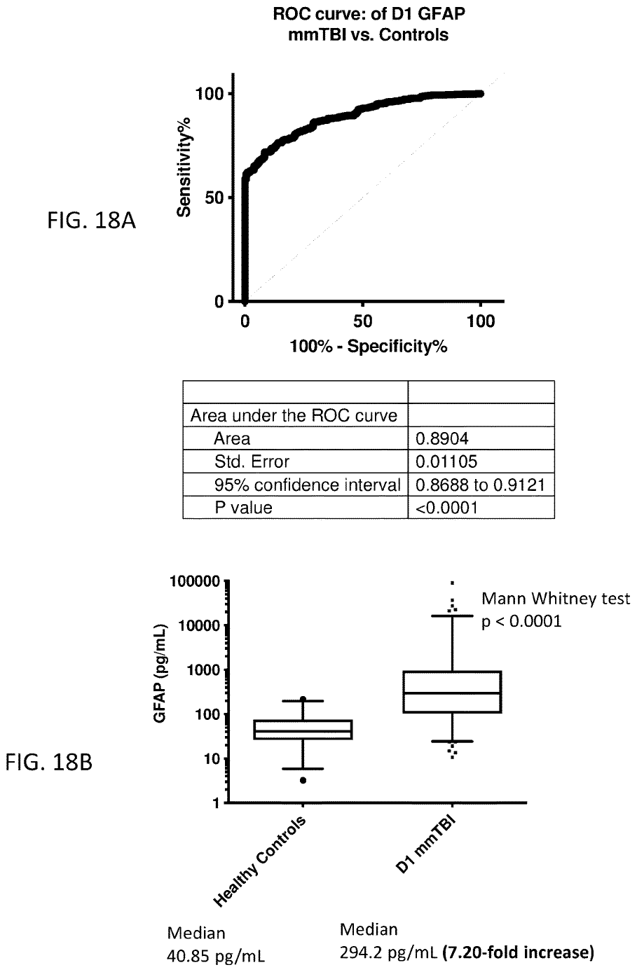

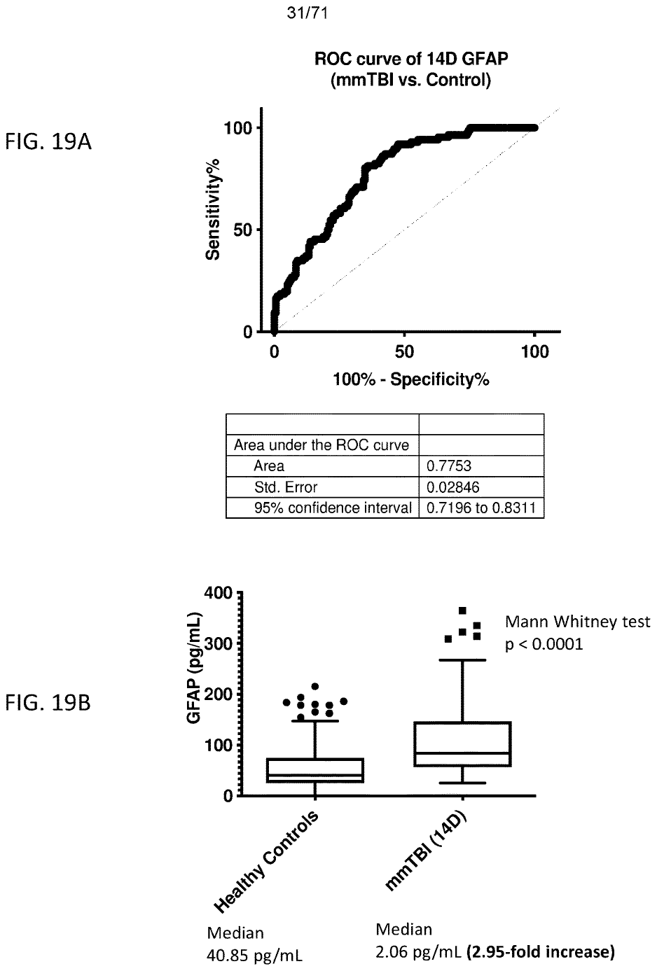

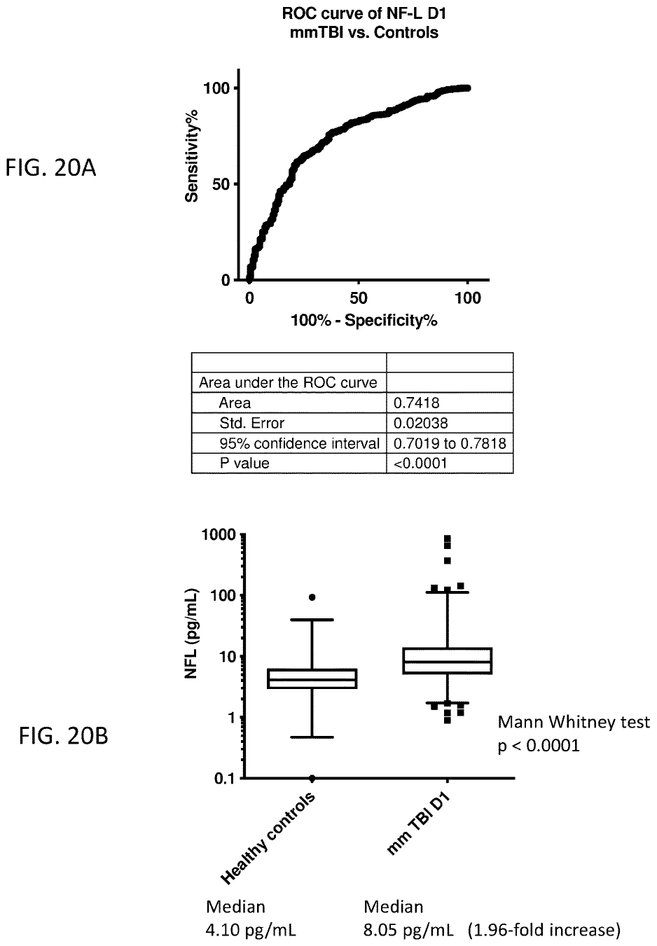

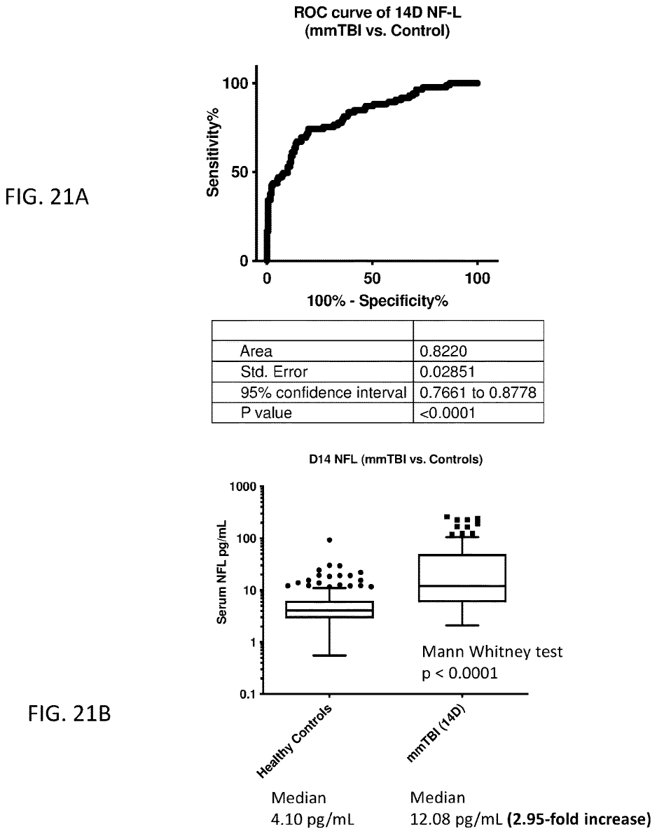

As mentioned above, glial fibrillary acidic protein (GFAP) is one of the most robust markers that can detect mild TBI with anatomical lesions (Czeiter, Amrein et al. 2020, Wang, Kobeissy et al. 2021). The temporal profile of GFAP is extensively characterized as shown in FIG. 14A. A rapid decay of GFAP over time is clearly evident in the GFAP ratio to D1 plot in FIG. 14B, albeit low levels of elevated GFAP do persistent over the sub-acute period following TBI, above the normative baseline levels as depicted by the arrow (60 μg/mL), as shown in FIG. 14A. FIG. 15 further shows that in wet plasma, GFAP elevations is mainly in day 1, but not 2 week or 6 month post-injury. In contrast, FIGS. 16A, 16B, and 16C show that NF-L measured with a conventional platform all showed that its mean 2 week levels are the highest as compared to that of healthy control, regardless of if it is detected from serum or plasma samples. Similarly, pNF-H, measured with Ella platform shows its larger mean elevations at 2 week post-injury. FIGS. 18A and 18B show that D1 GFAP is strongest in differentiating moderate-mild TBI (mmTBI) from healthy controls with the area under the ROC (AUC) of 0.8904; but its diagnostic accuracy declines to only AUC of 0.7753 (FIGS. 19A, 19B). In contrast, NF-L is only a fair differentiator of mmTBI from healthy control with AUC of 0.7418, yet, FIGS. 21A and 21B show that D14 post-injury serum NF-L levels rises to an AUC of 0.8220. Thus, this is another example that it is advantageous in the present invention to measure more than one brain injury biomarker at more than one time point.

Through the measurement of the high specificity neuroactive biomarker MOG from a subject in combination with values obtained from the high sensitivity-low neuroactive selectivity secondary neuroactive biomarkers outlined above, a determination of subject neurological condition is provided with greater specificity as to the presence of TBI and the degree of TBI. The severity of TBI is defined based on the Glasgow scale and spans a spectrum from severe through moderate to mild.

MOG has been found to be a reliable marker of brain injury in TBI. That is, there is a robust release of MOG into human serum from the acute, 1-10 days after injury, to the chronic, to days to 12 months after injury. Importantly, the MOG biomarker presents with chronic phase levels higher than acute levels, as shown in FIG. 4C, suggesting demyelination continues long after initial injury.

Evaluation of MOG as a marker of injury severity is accomplished by obtaining serial wet plasma specimens and DPS specimens from patients who have experienced head trauma after admission (with post-injury time recorded), 24 hours after admission, twice daily thereafter for up to 14 days, once at 30 days, and once at discharge (n=30 each of wet and dried specimens total up to discharge). Single wet plasma specimens and DPS are also collected from healthy control individuals.

In patients with favorable outcomes, slightly increased initial levels of MOG return to normal within 3 to 4 days. However, in patients with unfavorable outcomes, initial levels are markedly increased, with a tendency to gradually decrease only after day 20. As such, MOG is reliable in clinical severe TBI for which outcomes are poor. No correlative increase in MOG has been previously observed in the absence of severe TBI. In contrast to severe injuries which are relatively easy to diagnose, minor head injury is usually defined as a clinical state involving a Glasgow Coma Scale (GCS) score of 13-15; the lower the score the more severe the injury. In contrast to prior art attempts at using other biomarkers as a standalone biomarker, the inventors surprisingly discovered that the detection of MOG at modestly elevated levels along or in combination with changes in levels of secondary biomarkers synergistically allows one to distinguish and diagnose mild and moderate forms of traumatic brain injury allowing a physician to determine which subjects are more likely to require intensive therapy. As such, a first biomarker as used herein is illustratively MOG. Again as mentioned about MOG released into the circulation might trigger immune response by producing MOG antibody IgG and IgM. Such autoimmune response can potential lead to MOGAD or MOGAD-like disorders or symptoms, as the autoimmune attack of myelin sheath. Thus, again this argues for the uniqueness and unobvious advantage of measuring MOG as a brain injury biomarkers over time. Further, it might be also important to concurrently measure the anti-MOG autoantibody IgG and IgM levels in the same brain injury serial samples to further inform on the clinical recovery and autoimmunity status of the patient.

UCH-L1 (neuronal cell body damage marker) has a high degree of specificity for trauma that if measured in conjunction with MOG provides more meaningful clinical information as to the nature and extent of the injury involved than the mere measure of MOG alone. The nature of the UCH-L1 biomarker is detailed in U.S. Pat. Nos. 7,291,710 and 7,396,654, the contents of which are hereby incorporated by reference.

It is appreciated that MOG is a synergistic biomarker when used in combination with one or more additional biomarkers. Illustratively, the quantity of a second biomarker is determined in the same sample or in a second biological sample obtained at the same time, at an earlier time, or at a later time than that when the first biological sample was obtained. A second biomarker is illustratively MOG antibody IgG and IgM, UCH-L1; GFAP, Vimentin, pNF-H, MBP, NFL; Tau; P-Tau; alpha II-spectrin breakdown products (SBDP): SBDBP150N, SBDP150, SBDP145, SBDP150i, SBDP120; MAP2; VAMP5; WASF1; CAMKK1; BDNF; or additional combinations thereof. In some embodiments three biomarkers are detected including MOG, a second biomarker, and a third biomarker. A third biomarker is illustratively UCH-L1; GFAP; NFL; Tau; P-Tau; alpha II-spectrin breakdown products (SBDP): SBDBP150N, SBDP150, SBDP145, SBDP150i, SBDP120; MAP2; Vimentin; VAMP5; WASF1; CAMKK1; BDNF; or additional combinations thereof. It is appreciated that when a third biomarker is present that it is a different biomarker than a first biomarker or a second biomarker. A second biomarker and a third biomarker are not MOG. A difference is a different protein, a different cleavage product, a different dimerization state, or a different modification such as but not limited to phosphorylation state, glycosylation state, or other recognized modification.

It is appreciated that in other inventive embodiments, a ratio of P-Tau; Tau is synergistic when used in combination with one or more additional biomarkers as a detection panel. P-Tau 181 being a particularly advantageous phosphorylation site of P-Tau. The quantity of a second biomarker is determined in the same sample or in a second biological sample obtained at the same time, at an earlier time, or at a later time than that when the first biological sample was obtained. A second biomarker is illustratively includes MOG, MOG antibody IgG and IgM, GFAP, GFAP breakdown products (BDPS); interleukins, pNF-H, MBP, NF-L; MAP2; or combinations thereof. In some embodiments the second biomarker combination includes GFAP, and at least two MOG, MOG antibody IgG and IgM, IL-6, IL-15, pNF-H, MBP, NF-L, MAP2, or additional combinations thereof. In still other embodiments, the second biomarker combination includes GFAP or BDPS, IL-6, IL-15, and NF-L

The recognition of the above combinations as novel and unexpectedly powerful biomarker panel for neuronal injury such as TBI or stroke reveals, MS or Alexander disease, the importance of several associations identified by the inventors between these biomarkers as illustrated in Table 1.

TABLE 1
First set of inventive biomarker panels

| Novel Neural injury and neurological | |
|---|---|
| condition diagnostic biomarker | |
| pairing/panel | Information |
| P-Tau:Tau + MOG | Tau is a neuronal protein which helps |
| | stabilize microtubules in the axon. The paring |
| | allows monitoring of both axon health and myelin |
| | sheath/white matter health. Furthermore, P-Tau |
| | or hyperphosphorylated Tau or P-Tau/T-Tau ratio |
| | in blood is indicative of the |
| | neurodegenerative/tauopathy tendency, risk or |
| | progression. MOG (a glycoprotein exclusively |
| | expressed on the surface of the myelin sheath in |
| | the central nervous system (CNS) and is |
| | indicative of myelin damage and/or white matter |
| | injury. Thus, this paring allows the monitoring the |
| | myelin sheath health with specific implications in |
| | Alexander disease. |
| P-Tau:Tau + MOG antibody IgG and/or | Anti-MOG antibody or autoantibody as |
| IgM | IgG or IgM isoforms could be triggered as |
| | continuing and sustained release in MOG from |
| | the injured brain, especially in a subpopulation of |
| | vulnerable or immuno-hypersensitive patients. |
| | Thus, concurrently measuring both blood levels |
| | of MOG antigen levels and its MOG antibody IgG |
| | and IgM gives the clinicians the best assessment |
| | of possible MOGAD development risk and help |
| | tailor immunosuppression or other treatment |
| | options. |
| P-Tau: Tau + GFAP | GFAP is a glia protein and localizes |
| | subcellularly; For GFAP information see PCT- |
| | US2009-053376. This pairing allows monitoring |
| | of the myelin sheath/white matter health, and |
| | detection of Alexander diesase |
| P-Tau: Tau + MAP2 | MAP2 (neuronal dendritic marker) paring |
| | allows monitoring of both neuronal structural and |
| | myelin sheath/white matter health and improves |
| | diagnostic accuracy. See Hayes et al. (2008) U.S. |
| | Pat. No. 7,456,027 B2 |
| P-Tau: Tau + NFL and pNF-H | NFL and pNF-H are biomarkers in |
| | neurodegenerative discases, which provides a |
| | sensitive measurement of neuroaxonal damage, |
| | regardless of cause. Indicative of axonal injury. |
| | The paring allows monitoring of both neuronal |
| | structural (axonal) and myelin sheath/white |
| | matter health. |
| P-Tau:Tau + IL-6/IL-15 | Interleukin-6 (IL-6) in particular is |
| | indicative of a degree of neuroinflammation. IL- |
| | 15 stimulates CD8 T cell and natural killer (NK) |
| | cell activation, proliferation, and cytolytic activity |
| | and therefore is indicative of antitumor responses. |
| | Resulting in a combination that helps in |
| | differential diagnosis as to infection, immune |
| | cascade, and tumor detection. |

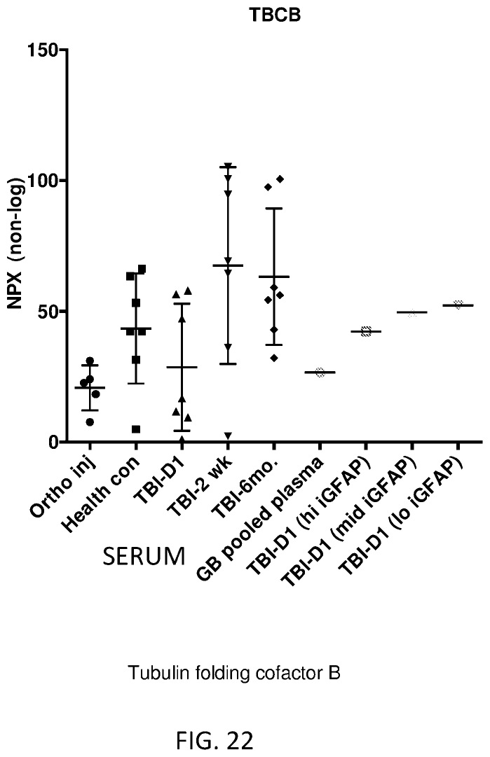

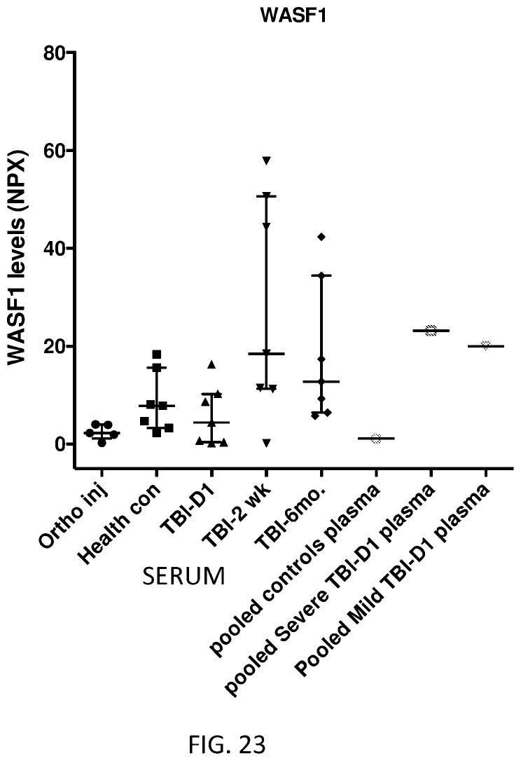

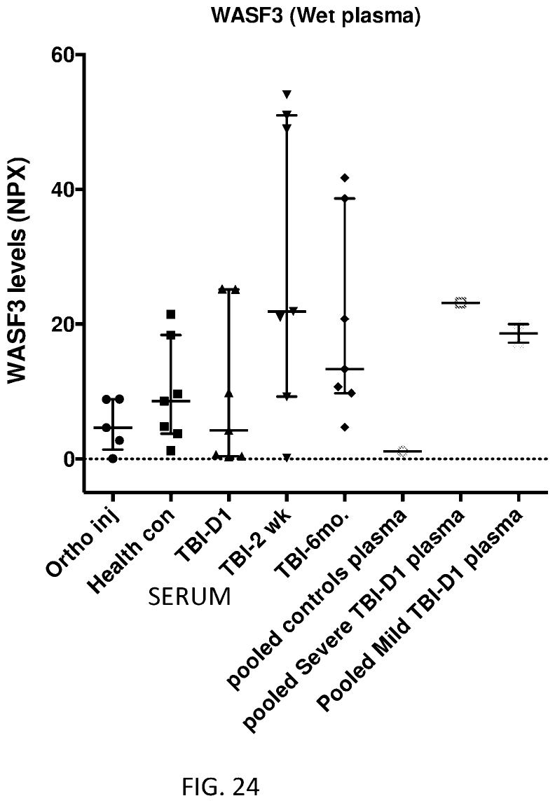

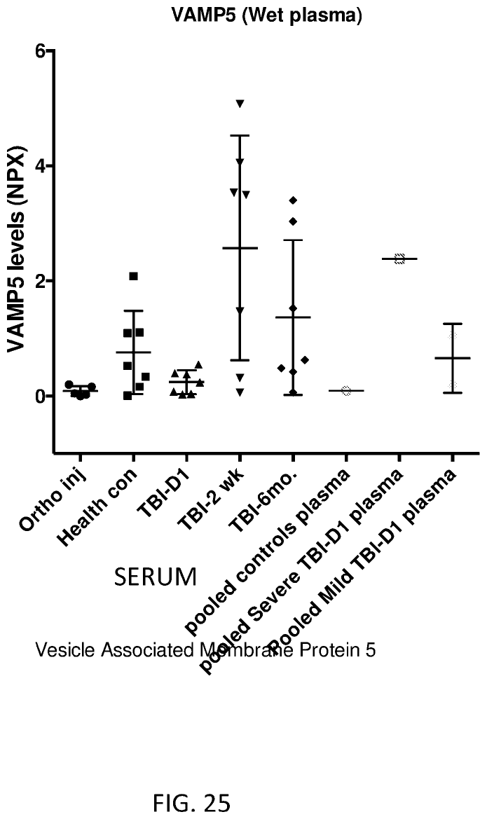

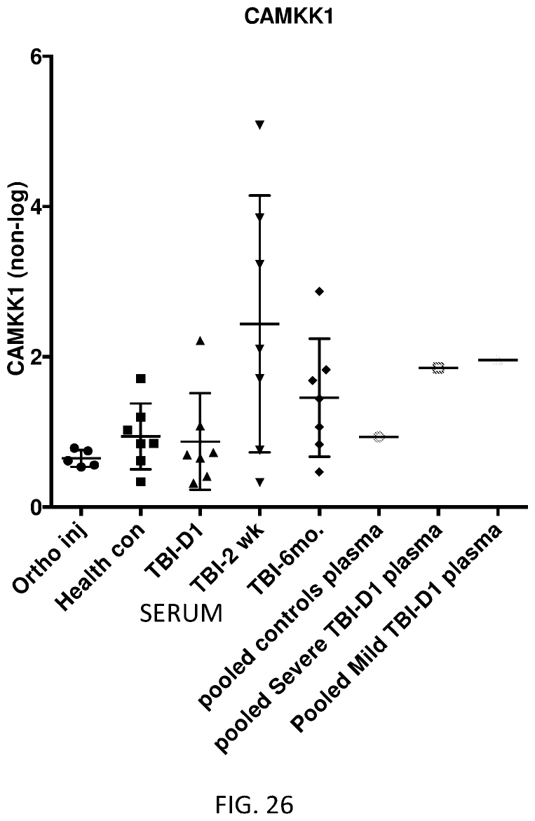

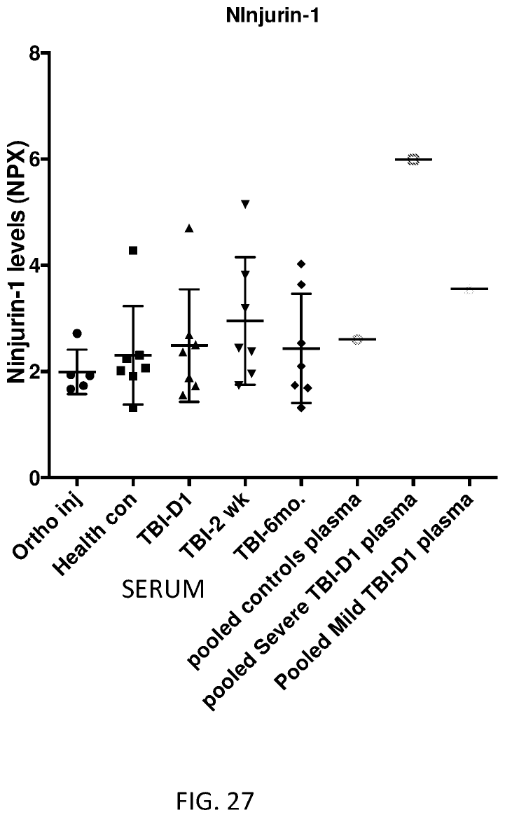

As illustration of the usefulness of the possible complementary biomarkers to MOG, FIG. 22 shows that tubulin folding cofactor B (TBCB), which is enriched in the brain has elevated 2 week and 6 mo. blood levels. FIGS. 23-26 shows synaptic markers WASF1, WAF3, VAMP5, postsynaptic density marker CAMKK1, respectively, have peak blood elevations in 2 week and slightly declined but still elevated levels at 6 mo. post-injury. FIG. 27 shows that mean serum Injurin-1 levels are elevated most pronounced at 2 week post-injury when compared to TBI D1 or normal controls levels. Yet with pooled plasma D1 TBI samples is about 3-fold higher than control plasma levels. The temporal profile revealed for these possible complementary biomarkers to MOG as quantified by a conventional platform screening of over about 400 proteins.

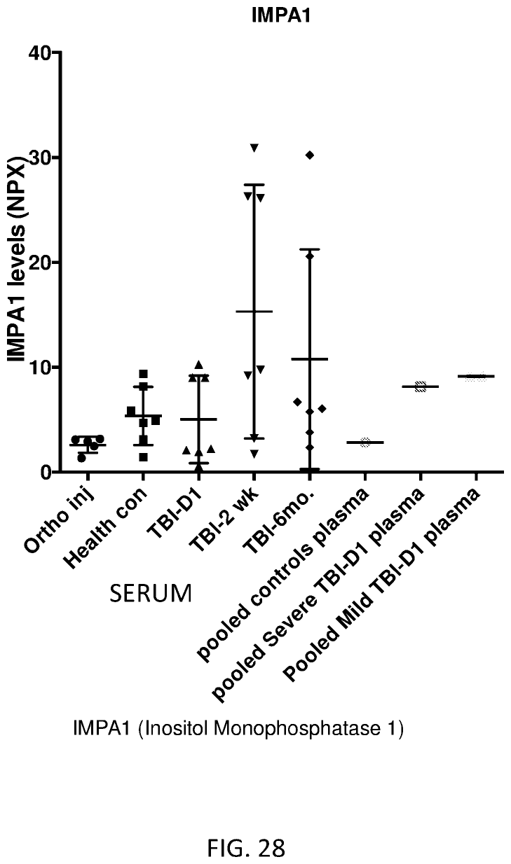

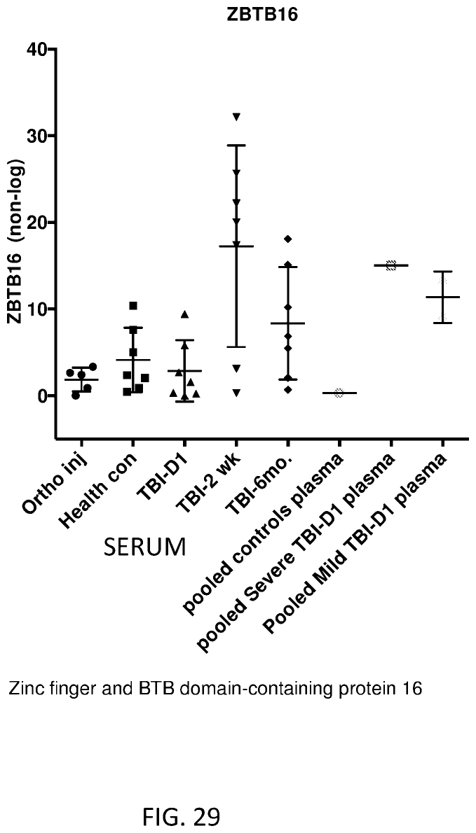

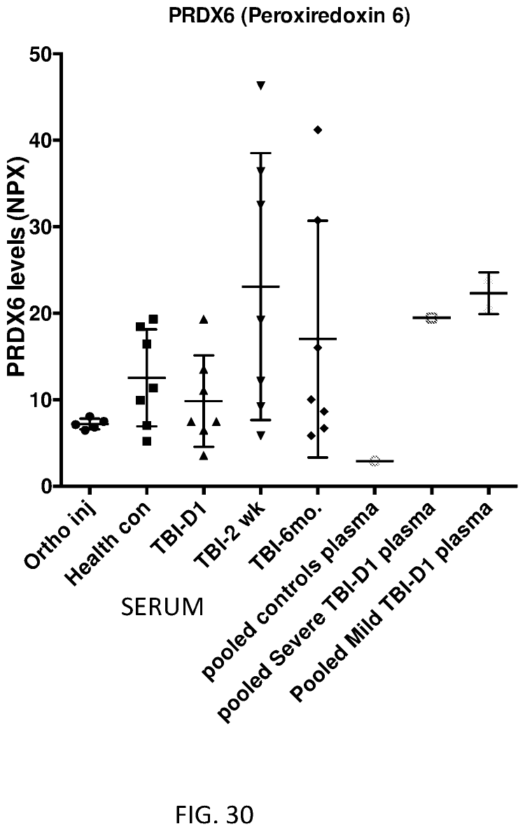

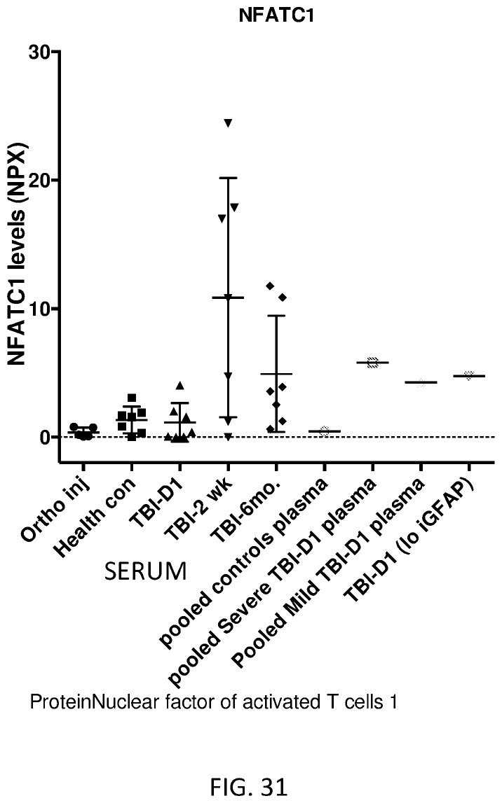

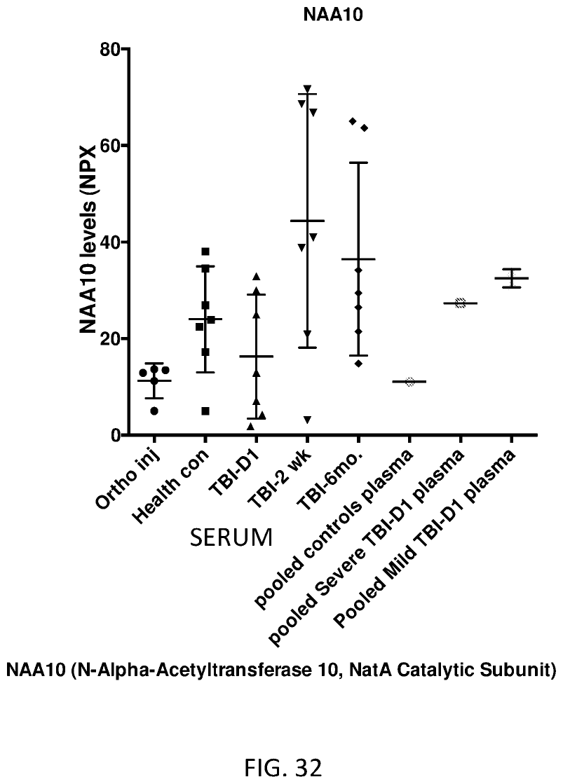

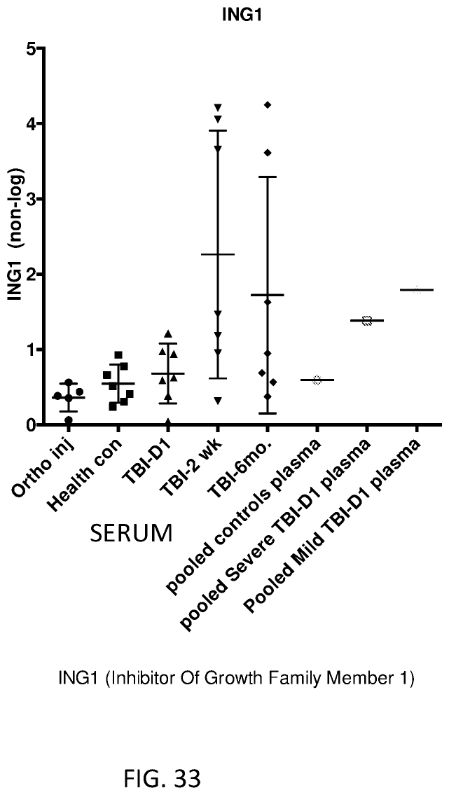

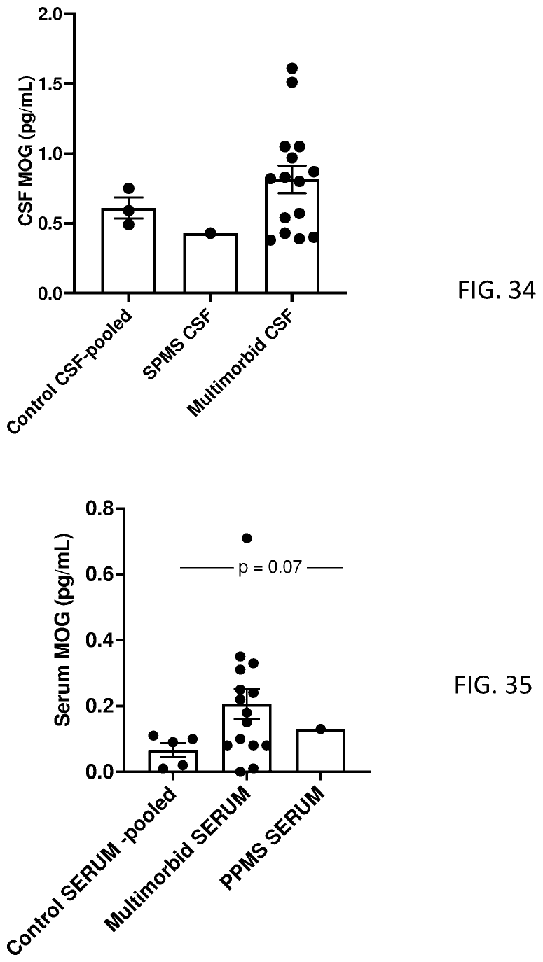

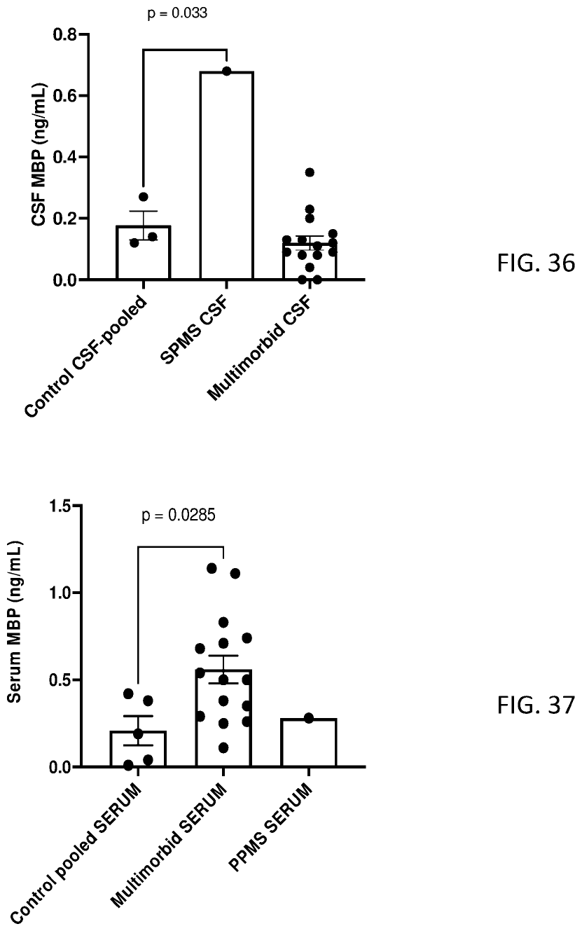

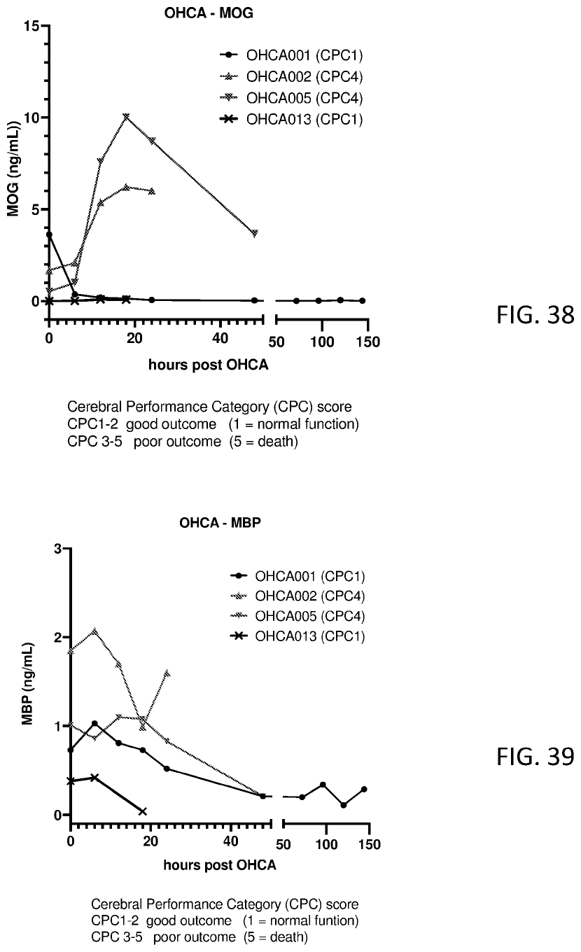

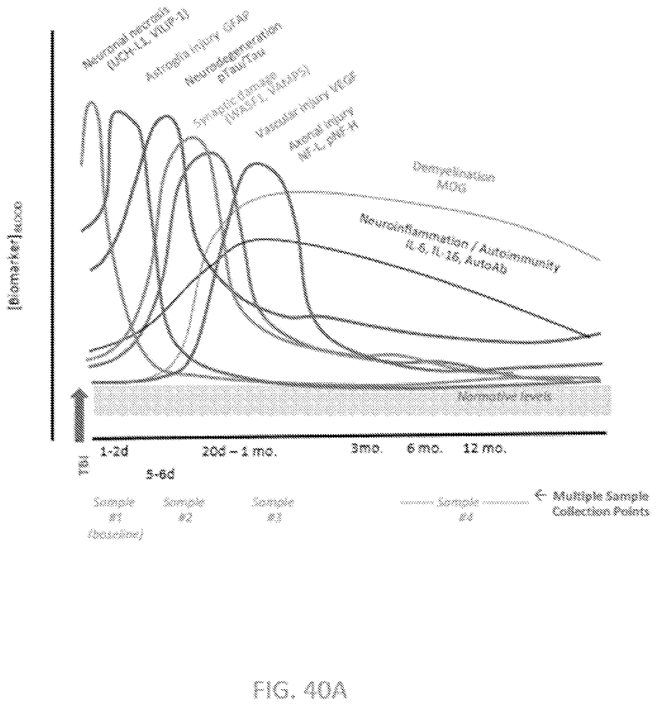

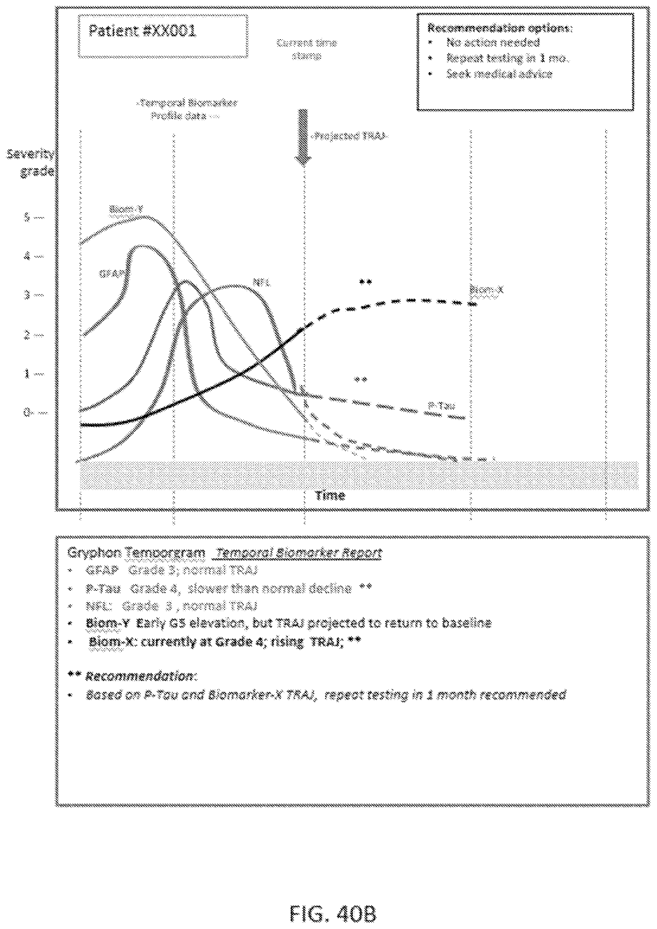

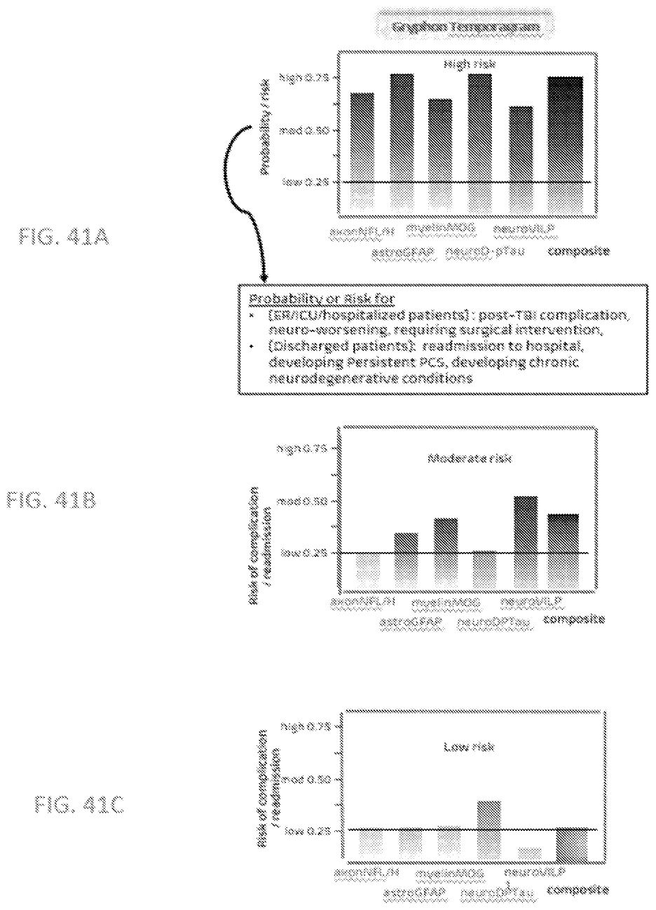

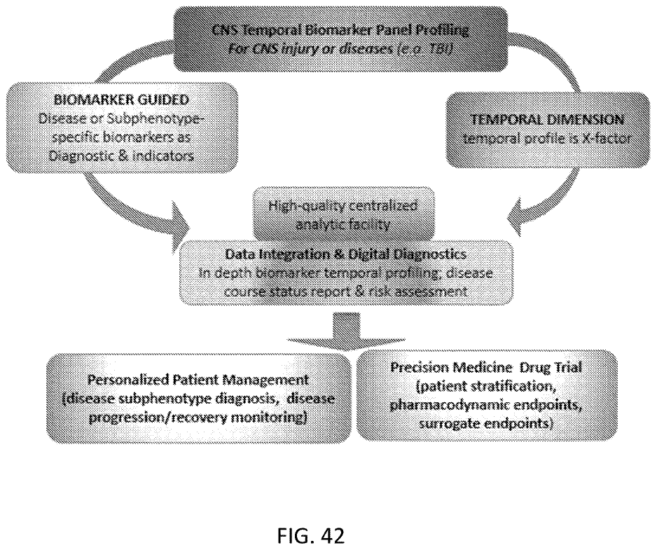

FIGS. 28, 29, 30, 31, 32, 33 further show the temporal profile of other possible complementary biomarkers to MOG as quantified by the conventional platform for over about 400 proteins. This set of markers are inositol monophosphatase 1 (IMPA1) (FIG. 28), zinc finger and BTB domain containing protein 16 (ZBTB16) (FIG. 29), Peroxiredoxin 6 (PRDX6) (FIG. 30), Nuclear factor of activated T cell 1 (NFATC1) (FIG. 31), N-alpha acetyltransderase 10 (NAA10) (FIG. 32) and Inhibitor of growth family member 1 (ING1) (FIG. 33), all show robust mean 2 week and 6 mo. post-TBI elevations, respectively.

The recognition of the above combinations as novel and unexpectedly powerful biomarkers for neuronal injury such as TBI or stroke reveals the importance of several associations identified by the inventors between these biomarkers as illustrated in Table 1.

TABLE 2
First set of inventive biomarker pairing/panels

| Novel Neural injury and neurological | |
|---|---|
| condition diagnostic biomarker | |
| pairing/pancl | Information |
| MOG + UCH-L1 | MOG (a glycoprotein exclusively |
| | expressed on the surface of the myelin sheath in |
| | the central nervous system (CNS) and is |
| | indicative of myelin damage and/or white matter |
| | injury. UCH-L1 is enriched in all neurons in the |
| | brain and localizes to neuronal cell body |
| | cytoplasma. Thus, this paring allows the |
| | monitoring both neuronal cell body health and |
| | myelin sheath health and to improve diagnostic |
| | accuracy. For UCH-L1 information see Hayes et |
| | al. (2008) U.S. Pat. No. 7,396,654 B2. |
| MOG + GFAP | GFAP is a glia protein and localizes |
| | subcellularly; For GFAP information see PCT- |
| | US2009-053376. This pairing allows monitoring |
| | of both astroglia health and myelin sheath/white |
| | matter health, and detection of Alexander disease |
| MOG + UCH-L1 + GFAP | MOG + GFAP + UCH-L1 triple |
| | combination improves diagnostic accuracy. The |
| | panel also allow concurrent assessment of neuron, |
| | astrocyte and oligodendrocyte/myelin integrity or |
| | injury. |
| MOG + one of the alpha II-spectrin | MOG + SBDP paring allows monitoring of |
| breakdown products (SBDP): | both neuronal structural (axonal) and myelin |
| SBDBP150N, SBDP150, SBDP145, | sheath/white matter health and improves |
| SBDP150i, SBDP120 | diagnostic accuracy. Alpha II-spectrin is an |
| | axonally enriched marker and its SBDP are |
| | produced by protease activation (calpain, |
| | caspase): SBDBP150N (Sequence X-QQQEVY- |
| | CO₂H), SBDP150 (NH₂-GMMPR-X), SBDP145 |
| | (NH₂-SAHEVQR-X), SBDP150i (NH2- |
| | SKTASPW-X), SBDP120 (sequence NH₂- |
| | SVEAL-X); where X = 0-5 any additional amino |
| | acid. Sequence based on Human Alpha II- |
| | Spectrin II (nonerythroid) protein accession # |
| | A3571; For additional information regarding |
| | SBDP see Hayes et al. (2007) U.S. Pat. No. |
| | 7,291,710 B2 |
| MOG + MAP2 | MOG + MAP2 (neuronal dendritic marker) |
| | paring allows monitoring of both neuronal |
| | structural and myelin sheath/white matter health |
| | and improves diagnostic accuracy. See Hayes et |
| | al. (2008) U.S. Pat. No. 7,456,027 B2 |
| MOG + Vimentin | Vimentin is glia a protein and localizes |
| | subcellularly. Vimentin is a Type III filament in |
| | glia. Vimentin is a novel neural injury, |
| | neurological condition biomarker. This pairing |
| | allows monitoring of both glia health and myelin |
| | sheath/white matter health. |
| MOG + NFL and pNF-H | NFL and pNF-H are biomarkers in |
| | neurodegenerative diseases, which provides a |
| | sensitive measurement of neuroaxonal damage, |
| | regardless of cause. Indicative of axonal injury. |
| | The paring allows monitoring of both neuronal |
| | structural (axonal) and myelin sheath/white |
| | matter health. |
| MOG + VAMP5 and/or other VAMP2, | VAMP5 and VAMP2 and VAMP3 are |
| other VAMP, SNAP23, SNAP25 and | proteins associated with vascular transport and |
| synapsin isoforms | synaptic function, and SNAP25, SANP23 and |
| | synapsin isoforms are associated with presynaptic |
| | terminal and neurotransmitter vesicle docking |
| | mechanism. The paring with MOG allows |
| | monitoring of both synaptic injury and myelin |
| | sheath/white matter health. |
| MOG + WASF1 and/or WASF3 | Wiskott-Aldrich syndrome protein family |
| | member WASFI and WASF3 are enriched in |
| | brain and localized specifically in neuronal cell |
| | body cytoplasma. They associated with |
| | promotion of formation of actin filaments and is |
| | also associated in APP protein trafficking to the |
| | cell surface. The release of WASFI or WASF3 |
| | into biofluids are indicative of neuronal cell body |
| | injury and cell neuronal damage or cell death. The |
| | paring of WASF with MOG allows monitoring of |
| | both actin filament and synaptic health and |
| | myelin sheath/white matter health. |
| MOG + Tau and/or P-Tau | Scc Table 1 |
| MOG + CAMKK1 and/or CAMKII | CAMKK1 and CAMPKII are signaling |
| | proteins for postsynaptic density for maintaining |
| | synaptic health. The paring allows monitoring of |
| | both postsynaptic density health and myelin |
| | sheath/white matter health. |
| MOG + BDNF and/or pro-BDNF | BDNF and its pro-form pro-BDNF signals |
| | for neurorecovery, neurorestoration. The paring |
| | allows monitoring of both neurorecovery and |
| | neurorestoration health and myelin sheath/white |
| | matter health. |
| MOG + MOG antibody IgG and/or IgM | Anti-MOG antibody or autoantibody as |
| | IgG or IgM isoforms could be triggered as |
| | continuing and sustained release in MOG from |
| | the injured brain, especially in a subpopulation of |
| | vulnerable or immuno-hypersensitive patients. |
| | Thus, concurrently measuring both blood levels |
| | of MOG antigen levels and its MOG antibody IgG |
| | and IgM gives the clinicians the best assessment |
| | of possible MOGAD development risk and help |
| | tailor immunosuppression or other treatment |
| | options. |

In some embodiments of the present invention, a first biomarker is MOG and a second biomarker is GFAP.

In other inventive embodiments, Glial Fibrillary Acidic Protein (GFAP) is detected in a biological sample along with UCH-L1 and MOG. GFAP, as a member of the cytoskeletal protein family, is the principal 8-9 nanometer intermediate filament glial cells such as in mature astrocytes of the central nervous system (CNS). GFAP is a monomeric molecule with a molecular mass between 40 and 53 kDa and an isoelectric point between 5.7 and 5.8. GFAP is highly brain specific protein that is not found outside the CNS under normal physiological conditions. GFAP is released in response to neurological insult and released into the blood and CSF soon thereafter. In the CNS following injury, either as a result of trauma, disease, genetic disorders, or chemical insult, astrocytes become reactive in a way termed astrogliosis or gliosis that is characterized by rapid synthesis of GFAP. It is appreciated that GFAP is optionally detected as a monomer or as a multimer such as a dimer.

Any subject that expresses an inventive biomarker is operable herein. Illustrative examples of a subject include a dog, a cat, a horse, a cow, a pig, a sheep, a goat, a chicken, non-human primate, a human, a rat, a mouse, and a cell. Subjects who benefit from the present invention are illustratively those suspected of having or at risk for developing abnormal neurological conditions, such as victims of brain injury caused by traumatic insults (e.g., gunshot wounds, automobile accidents, sports accidents, shaken baby syndrome), and ischemic events (e.g., stroke, cerebral hemorrhage, cardiac arrest).

The inventive neuroactive biomarker analyses of MOG and one or more additional biomarkers are illustratively operable to detect and diagnose TBI of all degrees from severe to mild, owing to the specificity of a second or third biomarker and the higher degree of sensitivity associated with MOG.

In vivo or in vitro screening or assay protocols illustratively include measurement of a neuroactive biomarker in a biological sample obtained from a subject.

Studies to determine or monitor levels of neuroactive biomarker levels of MOG and one or more additional biomarkers are optionally combined with behavioral analyses or motor deficit analyses such as: motor coordination tests illustratively including Rotarod, beam walk test, gait analysis, grid test, hanging test and string test; sedation tests illustratively including those detecting spontaneous locomotor activity in the open-field test; sensitivity tests for allodynia-cold bath tests, hot plate tests at 38° C. and Von Frey tests; sensitivity tests for hyperalgesia-hot plate tests at 52° C. and Randall-Sellito tests; and EMG evaluations such as sensory and motor nerve conduction, Compound Muscle Action Potential (CMAP) and h-wave reflex.

An exemplary process for detecting the presence or absence of MOG and a second biomarker in one or more biological samples involves obtaining a biological sample from a subject, such as a human, contacting the biological sample with an agent capable of detecting of the marker being analyzed, illustratively including an antibody or nucleic acid probe, and analyzing binding of the agent optionally after washing. Those samples having specifically bound agent (or reduced levels thereof in a competitive assay) express the marker being analyzed.

To provide correlations between neurological condition and measured quantities of MOG and one or more additional biomarkers, samples of CSF or serum are collected from subjects with the samples being subjected to measurement of MOG and one or more additional biomarkers. The subjects vary in neurological condition. Detected levels of biomarkers are then optionally correlated with CT scan results as well as GCS scoring. Based on these results, an inventive assay is developed and validated such as by the methods of Lee et al., Pharmacological Research 23:312-328, 2006. It is appreciated that levels of biomarkers are obtained from one or more of many different types of biological sample. Neuroactive biomarker levels in addition to being obtained from biological samples such as CSF and serum, are also readily obtained from blood, plasma, saliva, urine, as well as solid tissue biopsy. While CSF is a commonly used sampling fluid owing to direct contact with the nervous system, it is appreciated that other biological fluids have advantages in being sampled for the same or other purposes and therefore allow for inventive determination of neurological condition optionally as part of a battery of tests performed on a single biological sample such as blood, plasma, serum, saliva or urine.

A biological sample is obtained from a subject by conventional techniques. For example, CSF is obtained by lumbar puncture. Blood is obtained by venipuncture, while plasma and serum are obtained by fractionating whole blood according to known methods. Surgical techniques for obtaining solid tissue samples are well known in the art. For example, methods for obtaining a nervous system tissue sample are described in standard neurosurgery texts such as Atlas of Neurosurgery: Basic Approaches to Cranial and Vascular Procedures, by F. Meyer, Churchill Livingstone, 1999; Stereotactic and Image Directed Surgery of Brain Tumors, 1st ed., by David G. T. Thomas, WB Saunders Co., 1993; and Cranial Microsurgery: Approaches and Techniques, by L. N. Sekhar and E. De Oliveira, 1st ed., Thieme Medical Publishing, 1999, the contents of each of which are incorporated herein by reference. Methods for obtaining and analyzing brain tissue are also described in Belay et al., Arch. Neurol. 58:1673-1678 (2001); and Seijo et al., J. Clin. Microbiol. 38:3892-3895 (2000), the contents of which are incorporated herein by reference.

A process as provided herein can be used to detect MOG and one or more additional biomarkers in a biological sample in vitro, as well as in vivo. The quantity of expression of MOG and one or more additional biomarkers in a sample is optionally compared with appropriate controls such as a first sample known to express detectable levels of the marker being analyzed (positive control) and/or a second sample known to not express detectable levels of the marker being analyzed (a negative control). For example, in vitro techniques for detection of a marker include enzyme linked immunosorbent assays (ELISAs), western blots, immunoprecipitation, and immunofluorescence. Also, in vivo techniques for detection of a marker illustratively include introducing a labeled agent that specifically binds the marker into a biological sample or test subject. For example, the agent can be labeled with a radioactive marker whose presence and location in a biological sample or test subject can be detected by standard imaging techniques.

Any suitable molecule that specifically binds MOG or one or more additional biomarkers or any suitable molecule that specifically binds one or more other neuroactive biomarkers are operative in the invention to achieve a synergistic assay. An exemplary agent for biomarker detection and quantification is an antibody capable of binding to the biomarker being analyzed. An antibody is optionally conjugated to a detectable label. Such antibodies can be polyclonal or monoclonal. An intact antibody, a fragment thereof (e.g., Fab or F(ab′)₂), or an engineered variant thereof (e.g., sFv) can also be used. Such antibodies can be of any immunoglobulin class including IgG, IgM, IgE, IgA, IgD and any subclass thereof. Other example of binding agents to MOG are single stranded DNA or RNA nucleic acid.

Antibody-based assays are illustratively used in analyzing a biological sample for the presence of biomarker. Suitable western blotting methods are optionally used. For more rapid analysis (as may be important in emergency medical situations), immunosorbent assays (e.g., ELISA and RIA) and immunoprecipitation assays may be used. As one example, the biological sample or a portion thereof is immobilized on a substrate, such as a membrane made of nitrocellulose or PVDF; or a rigid substrate made of polystyrene or other plastic polymer such as a microtiter plate, and the substrate is contacted with an antibody that specifically binds a second or additional biomarker and a second antibody specific for MOG under conditions that allow binding of antibody to the biomarker being analyzed. After washing, the presence of the antibody on the substrate indicates that the sample contained the marker being assessed. If the antibody is directly, or indirectly (via a hapten), conjugated with a detectable label, such as an enzyme, fluorophore, or radioisotope, the label presence is optionally detected by examining the substrate for the detectable label. Alternatively, a detectably labeled secondary antibody that binds the marker-specific antibody is added to the substrate. The presence of detectable label on the substrate after washing indicates that the sample contained the marker.

Numerous permutations of these basic immunoassays are also operative in the invention. These include the biomarker-specific antibody, as opposed to the sample being immobilized on a substrate, and the substrate is contacted with biomarker conjugated with a detectable label under conditions that cause binding of antibody to the labeled marker. The substrate is then contacted with a sample under conditions that allow binding of the marker being analyzed to the antibody. A reduction in the amount of detectable label on the substrate after washing indicates that the sample contained the marker. Other methods of biomarker detection operative herein include mass spectrometry and lateral flow immunoassays.

It is appreciated that measuring mRNA in a sample per the present invention may be used as a surrogate for detection of the level of the corresponding biomarker protein in the sample. Thus, any of the biomarkers or biomarker panels described herein can also be detected by detecting the appropriate RNA. By way of example one or more nucleic acid probe specific to the corresponding biomarker(s) are reacted with the histological or cytological sample and can serve as the nucleic acid target in a nucleic acid amplification method. Suitable nucleic acid amplification methods include, for example, PCR, q-beta replicase, rolling circle amplification, strand displacement, helicase dependent amplification, loop mediated isothermal amplification, ligase chain reaction, and restriction and circularization aided rolling circle amplification. Non-amplification based methods for biomarkers can also be employed, including DNA or RNA nucleic acid for protein biomarker targets, as described below.

Although antibodies are preferred for use in the invention because of their extensive characterization, any other suitable agent (e.g., a peptide, a nucleic acid probe, or a small organic molecule) that specifically binds a biomarker is optionally used in place of the antibody in the above-described immunoassays. Aptamers are nucleic acid-based molecules that bind specific ligands, including protein biomarkers. Methods for making aptamers with a particular binding specificity are known as detailed in U.S. Pat. Nos. 5,475,096; 5,670,637; 5,696,249; 5,270,163; 5,707,796; 5,595,877; 5,660,985; 5,567,588; 5,683,867; 5,637,459; and 6,011,020, the contents of each of which are incorporated herein by reference.

A myriad of detectable labels are operative in a diagnostic assay for biomarker expression and are known in the art. Labels and labeling kits are commercially available optionally from Invitrogen Corp, Carlsbad, CA. Agents used in methods for detecting a neuroactive biomarker are optionally conjugated to a detectable label, e.g., an enzyme such as horseradish peroxidase. Agents labeled with horseradish peroxidase can be detected by adding an appropriate substrate that produces a color change in the presence of horseradish peroxidase. Several other detectable labels that may be used are known. Common examples include alkaline phosphatase, horseradish peroxidase, fluorescent molecules, luminescent molecules, colloidal gold, magnetic particles, biotin, radioisotopes, and other enzymes.

The present invention employs a step of correlating the presence or amount of MOG and one or more additional biomarkers in a biological sample with the severity and/or type of TBI. The amount of UCH-L1, for example, and MOG in the biological sample is associated with neurological condition for traumatic brain injury such as by methods detailed in the examples. The results of an inventive assay to synergistically measure MOG and one or more additional biomarkers can help a physician, veterinarian, or scientist determine the type and severity of injury with implications as to the types of cells that have been compromised. These results are in agreement with CT scan and GCS results, yet are quantitative, obtained more rapidly, and at far lower cost.

An assay or process optionally provides a step of comparing the quantity of MOG and one or more additional biomarkers to normal levels of one or each to determine the neurological condition of the subject. The practice of an inventive process provides a test which can help a physician determine suitable therapeutics to administer for optimal benefit of the subject.

An assay for analyzing cell damage in a subject is also provided. The assay includes: (a) a substrate for holding a sample isolated from a subject suspected of having a damaged nerve cell, the sample being a fluid in communication with the nervous system of the subject prior to being isolated from the subject; (b) a MOG specific binding agent specific binding agent; (c) a second biomarker specific binding agent; and optionally (d) printed instructions for reacting: the second biomarker specific agent with the biological sample or a portion of the biological sample to detect the presence or amount of the second biomarker, and the agent specific for MOG with the biological sample or a portion of the biological sample to detect the presence or amount of MOG and the second biomarker in the biological sample. The inventive assay can be used to detect neurological condition for financial renumeration. In some embodiments a third biomarker specific agent is included that is specific for a third biomarker that is different than a second biomarker and is not MOG.

Baseline levels of biomarkers are those levels obtained in the target biological sample in the species of desired subject in the absence of a known neurological condition. These levels need not be expressed in hard concentrations but may instead be known from parallel control experiments and expressed in terms of fluorescent units, density units, and the like. Typically, in the absence of a neurological condition, one or more biomarkers are present in biological samples at a negligible amount. However, UCH-L1 is a highly abundant protein in neurons. Determining the baseline levels of biomarkers illustratively including UCH-L1 or MOG protein as well as RNA in neurons, plasma, or CSF, for example, of particular species is well within the skill of the art. Similarly, determining the concentration of baseline levels of other biomarkers is well within the skill of the art. Baseline levels are illustratively the quantity or activity of a biomarker in a sample from one or more subjects that are not suspected of having a neurological condition.

The relative levels of MOG or one or more additional biomarkers are optionally expressed as a ratio to control, baseline, or known elevated biomarker levels. As used herein a “ratio” is either a positive ratio wherein the level of the target biomarker is greater than the target in a second sample or relative to a known or recognized baseline level of the same target. A negative ratio describes the level of the target as lower than the target in a second sample or relative to a known or recognized baseline level of the same target. A neutral ratio describes no observed change in target biomarker.

A neurological condition optionally results in or produces an injury. As used herein an “injury” is an alteration in cellular or molecular integrity, activity, level, robustness, state, or other alteration that is traceable to an event. Injury illustratively includes a physical, mechanical, chemical, biological, functional, infectious, or other modulator of cellular or molecular characteristics. An injury optionally results from an event. An event is illustratively, a physical trauma such as an impact (illustratively, percussive) or a biological abnormality such as a stroke resulting from blockade (ischemic) of a blood vessel. As such the term “traumatic brain injury” (TBI) is meant to describe injury to the brain as the result of an event such as percussion or other impact, or blockade of a blood vessel.

An injury is optionally a physical event such as a percussive impact. An impact is optionally the like of a percussive injury such as resulting to a blow to the head, the body, or combinations thereof that either leave the cranial structure intact or results in breach thereof. Experimentally, several impact methods are used illustratively including controlled cortical impact (CCI) at a 1.6 mm depression depth, equivalent to severe TBI in human. This method is described in detail by Cox, C D, et al., J Neurotrauma, 2008; 25(11):1355-65, the contents of which are incorporated herein by reference. It is appreciated that other experimental methods producing impact trauma are similarly operable.

An injury may also result from stroke. Ischemic stroke is optionally modeled by middle cerebral artery occlusion (MCAO) in rodents. UCH-L1 protein levels, for example, are increased following mild MCAO which is further increased following severe MCAO challenge. Mild MCAO challenge may result in an increase of biomarker levels within two hours that is transient and returns to control levels within 24 hours. In contrast, severe MCAO challenge results in an increase in biomarker levels within two hours following injury and may be much more persistent demonstrating statistically significant levels out to 72 hours or more.

A step of correlating the presence or amount of a biomarker in a biological sample with the severity and/or type of nerve cell (or other biomarker-expressing cell) toxicity is optionally provided. The amount of biomarker(s) in the biological sample directly relates to severity of neurological condition as a more severe injury damages a greater number of nerve cells which in turn causes a larger amoun of biomarker(s) to accumulate in the biological sample (e.g., CSF; serum). Illustratively, elevated levels of UCH-L1, GFAP, or both along with modestly elevated levels of MOG reveal severe TBI. Elevated UCH-L1, GFAP or both along with no appreciable increase in MOG can reveal moderate TBI. Absence of increases in MOG and one UCH-L1, GFAP or both following an impact reveal mild TBI. Also, the level of or kinetic extent of biomarkers present in a biological sample may optionally distinguish mild injury from a more severe injury. In an illustrative example, severe MCAO (2 h) produces increased UCH-L1 in both CSF and serum relative to mild challenge (30 min) while both produce UCH-L1 levels in excess of uninjured subjects. Moreover, the persistence or kinetic extent of the markers in a biological sample is indicative of the severity of the neurotoxicity with greater toxicity indicating increases persistence of UCH-L1 or MOG biomarkers in the subject that is measured in a process in biological samples taken at several time points following injury.

The invention optionally includes administration one or more compounds such as therapeutic agents or molecules being assayed for therapeutic or other potential that may alter one or more characteristics of a target biomarker such as concentration in a biological sample. A therapeutic optionally serves as an agonist or antagonist of a target biomarker or upstream effector of a biomarker. A therapeutic optionally affects a downstream function of a biomarker. For example, Acetylcholine (Ach) plays a role in pathological neuronal excitation and TBI-induced muscarinic cholinergic receptor activation may contribute to excitotoxic processes. As such, biomarkers optionally include levels or activity of Ach or muscarinic receptors. Optionally, an operable biomarker is a molecule, protein, nucleic acid or other that is affected by the activity of muscarinic receptor(s). As such, therapeutics operable in the subject invention illustratively include those that modulate various aspects of muscarinic cholinergic receptor activation.

Specific muscarinic receptors operable as therapeutic targets or modulators of therapeutic targets include the M₁, M₂, M₃, M₄, and M₅ muscarinic receptors.

The suitability of the muscarinic cholinergic receptor pathway in detecting and treating TBI arises from studies that demonstrated elevated ACh in brain cerebrospinal fluid (CSF) following experimental TBI (Gorman et al., 1989; Lyeth et al., 1993a) and ischemia (Kumagae and Matsui, 1991), as well as the injurious nature of high levels of muscarinic cholinergic receptor activation through application of cholinomimetics (Olney et al., 1983; Turski et al., 1983). Furthermore, acute administration of muscarinic antagonists improves behavioral recovery following experimental TBI (Lyeth et al., 1988a; Lyeth et al., 1988b; Lyeth and Hayes, 1992; Lyeth et al., 1993b; Robinson et al., 1990). As such chemical or biological agents such as compounds that bind to or alter a characteristic of a muscarinic cholinergic receptor are optionally screened for neurotoxicity of cells or tissues such as during target optimization in pre-clinical drug discovery.

A compound illustratively a therapeutic compound, chemical compound, or biological compound is illustratively any molecule, family, extract, solution, drug, pro-drug, or other that is operable for changing, optionally improving, therapeutic outcome of a subject at risk for or subjected to a neurotoxic insult. A therapeutic compound is optionally a muscarinic cholinergic receptor modulator such as an agonist or antagonist, an amphetamine. An agonist or antagonist may by direct or indirect. An indirect agonist or antagonist is optionally a molecule that breaks down or synthesizes acetylcholine or other muscarinic receptor related molecule illustratively, molecules currently used for the treatment of Alzheimer's disease. Cholinic mimetics or similar molecules are operable herein. An exemplary list of therapeutic compounds operable herein include: dicyclomine, scoplamine, milameline, N-methyl-4-piperidinylbenzilate NMP, pilocarpine, pirenzepine, acetylcholine, methacholine, carbachol, bethanechol, muscarine, oxotremorine M, oxotremorine, thapsigargin, calcium channel blockers or agonists, nicotine, xanomeline, BuTAC, clozapine, olanzapine, cevimeline, aceclidine, arecoline, tolterodine, rociverine, IQNP, indole alkaloids, himbacine, cyclostellettamines, derivatives thereof, pro-drugs thereof, and combinations thereof. A therapeutic compound is optionally a molecule operable to alter the level of or activity of a calpain or caspase. Such molecules and their administration are known in the art. It is appreciated that a compound is any molecule including molecules of less than 700 Daltons, peptides, proteins, nucleic acids, or other organic or inorganic molecules that is contacted with a subject, or portion thereof.

A compound is optionally any molecule, protein, nucleic acid, or other that alters the level of a neuroactive biomarker in a subject. A compound is optionally an experimental drug being examined in pre-clinical or clinical trials, or is a compound whose characteristics or affects are to be elucidated. A compound is optionally kainic acid, MPTP, an amphetamine, cisplatin or other chemotherapeutic compounds, antagonists of a NMDA receptor, any other compound listed herein, pro-drugs thereof, racemates thereof, isomers thereof, or combinations thereof. Example amphetamines include: ephedrine; amphetamine aspartate monohydrate; amphetamine sulfate; a dextroamphetamine, including dextroamphetamine saccharide, dextroamphetamine sulfate; methamphetamines; methylphenidate; levoamphetamine; racemates thereof; isomers thereof; derivatives thereof; or combinations thereof. Illustrative examples of antagonists of a NMDA receptor include those listed in Table 3 racemates thereof, isomers thereof, derivatives thereof, or combinations thereof:

TABLE 3

| | | |
|---|---|---|
| AP-7 (drug) | Gacyclidine | PEAQX |
| AP5 | Hodgkinsine | Perzinfotel |
| Amantadine | Huperzine A | Phencyclidine |
| Aptiganel | Ibogaine | 8A-PDHQ |
| CGP-37849 | Ifenprodil | Psychotridine |
| DCKA | Indantadol | Remacemide |
| Delucemine | Ketamine | Rhynchophylline |
| Dexanabinol | Kynurenic acid | Riluzole |
| Dextromethorphan | Lubeluzole | Sabeluzole |
| Dextrorphan | Memantine | Selfotel |
| Dizocilpine | Midafotel | Tiletamine |
| Eliprodil | Neramexane | Xenon |
| Esketamine | Nitrous oxide | |
| Ethanol | | |
| NEFA | | |

As used herein the term “administering” is delivery of a compound to a subject. The compound is a chemical or biological agent administered with the intent to ameliorate one or more symptoms of a condition or treat a condition. A therapeutic compound is administered by a route determined to be appropriate for a particular subject by one skilled in the art. For example, the therapeutic compound is administered orally, parenterally (for example, intravenously, by intramuscular injection, by intraperitoneal injection, intratumorally, by inhalation, or transdermally. The exact amount of therapeutic compound required will vary from subject to subject, depending on the age, weight and general condition of the subject, the severity of the neurological condition that is being treated, the particular therapeutic compound used, its mode of administration, and the like. An appropriate amount may be determined by one of ordinary skill in the art using only routine experimentation given the teachings herein or by knowledge in the art without undue experimentation.

Processes of detecting or distinguishing the magnitude of traumatic brain injury (TBI) is also provided. Traumatic brain injury is illustratively mild-TBI, moderate-TBI, or severe-TBI. As used herein mild-TBI is defined as individuals presenting with a CGS score of 12-15 or any characteristic described in the National Center for Injury Prevention and Control, Report to Congress on Mild Traumatic Brain Injury in the United States: Steps to Prevent a Serious Public Health Problem. Atlanta, GA: Centers for Disease Control and Prevention; 2003, incorporated herein by reference. Moderate-TBI is defined as presenting a GCS score of 9-11. Severe-TBI is defined as presenting a GCS score of less than 9, presenting with an abnormal CT scan or by symptoms including unconsciousness for more than 30 minutes, post traumatic amnesia lasting more than 24 hours, and penetrating cranial cerebral injury.

A process of detecting or distinguishing between mild- or moderate-TBI illustratively includes obtaining a sample from a subject at a first time and measuring a quantity of MOG and a second biomarker in the sample where an elevated MOG and second biomarker level indicates the presence of traumatic brain injury. The inventive process is optionally furthered by correlating the quantity of MOG and second biomarker with CT scan normality or GCS score. A positive correlation for mild-TBI is observed when the GCS score is 12 or greater, and neither MOG nor second biomarker levels are elevated. A positive correlation for moderate-TBI is observed when the GCS score is 9-11 and second biomarker levels are elevated with modest elevation of MOG returning to low levels within 24 hours of injury. Alternatively, or in addition, a positive correlation for moderate-TBI is observed when the CT scan results are abnormal, and second biomarker levels are elevated. Abnormal CT scan results are illustratively the presence of lesions. Unremarkable or normal CT scan results are the absence of lesions.

The levels of MOG and one or more additional biomarkers are optionally measured in samples obtained within 24 hours of injury. Illustratively, UCH-L1 and MOG levels are measured in samples obtained 0-24 hours of injury inclusive of all time points therebetween. In some embodiments, a second sample is obtained at or beyond 24 hours following injury and the quantity of MOG alone or along with additional biomarkers are measured.

Various aspects of the present invention are illustrated by the following non-limiting examples. The examples are for illustrative purposes and are not a limitation on any practice of the present invention. It will be understood that variations and modifications can be made without departing from the spirit and scope of the invention. While the examples are generally directed to mammalian tissue, specifically, analyses of rat tissue, a person having ordinary skill in the art recognizes that similar techniques and other techniques know in the art readily translate the examples to other mammals such as humans. Reagents illustrated herein are commonly cross reactive between mammalian species or alternative reagents with similar properties are commercially available, and a person of ordinary skill in the art readily understands where such reagents may be obtained.

### Example 1

Materials for Biomarker Analyses. Sodium bicarbonate, blocking buffer (Startingblock T20-TBS), Tris buffered saline with Tween 20 (TBST). Phosphate buffered saline (PBS); Tween 20; Ultra TMB ELISA; and Nunc maxisorp ELISA plates. Monoclonal and polyclonal UCH-L1 antibodies are made in-house or are obtained from Santa Cruz Biotechnology, Santa Cruz, CA. Antibodies directed to MOG are available from Santa Cruz Biotechnology, Santa Cruz, CA. Antibodies to GFAP are made in-house or are available from Santa Cruz Biotechnology, Santa Cruz, CA. Labels for antibodies of numerous subtypes are available from Invitrogen, Corp., Carlsbad, CA. Protein concentrations in biological samples are determined using bicinchoninic acid microprotein assays (Pierce Inc., Rockford, IL, USA) with albumin standards. All other necessary reagents and materials are known to those of skill in the art and are readily ascertainable.

Biomarker specific rabbit polyclonal antibodies and monoclonal antibodies are produced in the laboratory or are available from commercial sources known to those of skill in the art. To determine reactivity specificity of the antibodies a tissue panel is probed by western blot.

An indirect ELISA is used with recombinant biomarker protein attached to the ELISA plate to determine optimal concentration of the antibodies used in the assay. This assay determines suitable concentrations of biomarker specific binding agent to use in the assay. Microplate wells are coated with rabbit polyclonal antihuman biomarker antibody. After determining concentration of rabbit antihuman biomarker antibody for a maximum signal, maximal detection limit of the indirect ELISA for each antibody is determined. An appropriate diluted sample is incubated with a rabbit polyclonal antihuman biomarker antibody (capture antibody) for 2 hours and then washed. Biotin labeled monoclonal antihuman biomarker antibody is then added and incubated with captured biomarker. After thorough wash, streptavidin horseradish peroxidase conjugate is added. After 1 hour incubation and the last washing step, the remaining conjugate is allowed to react with substrate of hydrogen peroxide tetramethyl benzadine. The reaction is stopped by addition of the acidic solution and absorbance of the resulting yellow reaction product is measured at 450 nanometers. The absorbance is proportional to the concentration of the biomarker. A standard curve is constructed by plotting absorbance values as a function of biomarker concentration using calibrator samples and concentrations of unknown samples are determined using the standard curve.

ELISA is used to more rapidly and readily detect and quantitate UCH-L1 in biological samples in rats following CCI. For a UCH-L1 sandwich ELISA (swELISA), 96-well plates are coated with 100 μl/well capture antibody (500 ng/well purified rabbit anti-UCH-L1, made in-house by conventional techniques) in 0.1 M sodium bicarbonate, pH 9.2. Plates are incubated overnight at 4° C., emptied and 300 μl/well blocking buffer (Startingblock T20-TBS) is added and incubated for 30 min at ambient temperature with gentle shaking. This is followed by either the addition of the antigen standard (recombinant UCH-L1) for standard curve (0.05-50 ng/well) or samples (3-10 μl CSF) in sample diluent (total volume 100 μl/well). The plate is incubated for 2 hours at room temperature then washed with automatic plate washer (5×300 μl/well with wash buffer, TBST). Detection antibody mouse anti-UCH-L1-HRP conjugated (made in-house, 50 μg/ml) in blocking buffer is then added to wells at 100 μL/well and incubated for 1.5 h at room temperature, followed by washing. If amplification is needed, biotinyl-tyramide solution (Perkin Elmer Elast Amplification Kit) is added for 15 min at room temperature, washed then followed by 100 μl/well streptavidin-HRP (1:500) in PBS with 0.02% Tween-20 and 1% BSA for 30 min and then followed by washing. Lastly, the wells are developed with 100 μl/well TMB substrate solution (Ultra-TMB ELISA, Pierce #34028) with incubation times of 5-30 minutes. The signal is read at 652 nm with a 96-well spectrophotometer (Molecular Device Spectramax 190). Similar assays are performed using primary antibodies directed to S-100β and UCH-L1.

To specifically detect dimers of MOG, UCH-L1, or GFAP an ELISA assay is used where the capture and detection antibodies are directed to identical epitopes that are not involved in the dimerization of biomarker using similar techniques to those described by El-Agnaf OMA, et al, The FASEB Journal, 2006; 20:419-425, the contents of which are incorporated herein by reference. The above assay for UCH-L1 is repeated using 96-well plates coated with MOG antibody from Santa Cruz Biotechnology and blocked with blocking buffer (Startingblock T20-TBS) as described above. Samples (100 μL/well) are incubated with the plates for 2 hours at room temperature, followed by washing with an automatic plate washer (5×300 μl/well with wash buffer, TBST). Detection antibody is the identical antibody as the primary antibody but additionally conjugated with HRP (made in-house, 50 μg/ml), placed in blocking buffer and then added to wells at 100 μL/well and incubated for 1.5 h at room temperature, followed by washing. The wells are developed with 100 μl/well TMB substrate solution (Ultra-TMB ELISA, Pierce #34028) with incubation times of 5-30 minutes. The signal is read at 652 nm with a 96-well spectrophotometer (Molecular Device Spectramax 190). The assay allows specific detection of dimers. During assay development, identical samples are subjected to size exclusion chromatography as per are recognized methods and fractions are assayed by the single antibody ELISA. Positive results in higher molecular weight protein containing fractions are indicative of biomarker dimers.

### Example 2

The DPS sampling and the conventional immunoassay platform are combined with an evidence-based proprietary panel of blood-based temporal TBI protein biomarkers, including: the astrocytic biomarker GFAP; neuronal biomarkers neurofilament-light protein (NF-L) phosphorylated neurofilament-heavy (pNF-H), and phosphorylated microtubule associated protein tau (P-Tau); as well as the novel demyelination biomarker myelin oligodendrocyte glycoprotein (MOG); and cytokine measures of immune suppression/inflammation (e.g., IL-6). Spiked plasma/serum specimens are created with recombinant proteins as TBI biomarkers and pooled control/TBI specimens from both wet plasma/serum specimens and mock DPS specimens, generated by pipetting wet specimens onto the DPS sampling device's plasma collection disk, are examined. This (i) establishes authenticated gold-standards for the custom panel and positive/negative sample controls; (ii) standardizes preanalytical conditions such as the impact of different concentrations of detergents on the release of TBI biomarkers bound to hydrophobic proteins (e.g., albumin) for maximum recovery from DPS specimens from CP; (iii) determines the LLOQ, linear dynamic range, accuracy, precision, impact of matrix interference (e.g., EDTA), etc.; and (iv) establishes analytical comparability with archived wet specimens from historical TBI cohorts vs. mock DPS specimens thereof from moderate-to-severe TBI patients from the Intensive Care Unit (ICU), Emergency Medicine (EM), and hospitalized patients collected with up to daily frequency. To generate longitudinal mock DPS specimens, longitudinal serum specimens from CENTER-TBI are analyzed at 5-10 time points post-injury as a training set and more serum specimens at 4, 24, and 48 hours post-injury from the ProTECTIII/BioProTECT studies as a test set, where specimens from patients without extra-cranial injury are preferentially selected for enrichment. Longitudinal plasma specimens from the University of Pittsburgh serve as additional test/training sets. GFAP measurements on the iSTAT immunoassay platform serve as a benchmark and predicate for regulatory clearance in the future. The third immunoassay platform serves to confirm the “ground truth” levels of biomarkers in these specimens.

### Example 3

Striking preliminary data for the inventive SPS is generated using a prototype DPS sampling device from Capitainer (the Capitainer-P or CP) and the high sensitivity immunoassay platform from a conventional platform which, together with longitudinal data from traditional “wet” TBI specimens and immunoassay platforms, provides strong evidence for the success of the inventive DPS for Blood Testing of TBI Biomarkers.

First, data for Preliminary verification of the analytical comparability the high sensitivity immunoassay platform vs. the other immunoassay platforms is provided in FIGS. 9A and 9B. Briefly, 44 plasma samples from TBI subjects and healthy controls are analyzed in parallel for selected biomarkers from the inventive panel (e.g., NFL) on these three immunoassay platforms. A very strong correlation of wet platform NFL data to wet Ella NFL data (R2=0.861) is observed, as shown in FIG. 9A, and a good correlation of wet platform NFL data to wet third NFL data (R2=0.656) is observed, as shown in FIG. 9B. FIG. 9A is a graph showing preliminary verification of the analytical comparability of the platform high sensitivity immunoassay platform vs. the other immunoassay platforms with NFL measurements of de-identified, archived wet plasma specimens from full spectrum TBI subjects (1 dpi to 6 months) with the wet platform NFL data showing a very strong correlation with the other platform NFL data (R2=0.861). FIG. 9B is a graph showing preliminary verification of the analytical comparability of the high sensitivity immunoassay platform vs. the other immunoassay platforms with NFL measurements of de-identified, archived wet plasma specimens from full spectrum TBI subjects (1 dpi to 6 months) with the Platform NFL data showing a good correlation with third NFL data (R2=0.656).

Second, preliminary verification of the of the analytical comparability of wet vs. DPS for sampling is provided in FIGS. 10A and 10B. Briefly, 44 plasma samples from TBI subjects and healthy controls are spotted onto the DPS sampling device's collection disk, dried at room temperature, and stored for 24 hrs in desiccator to mimic real-world sampling and storage conditions. DPS proteins are then recovered from the collection disk with elution buffer and selected biomarkers from the inventive panel (e.g., NFL and WASF1) are assayed alongside their wet plasma counterparts. A very strong correlation of DPS Platform NFL data to wet Platform NFL data (R2=0.9531) is observed in FIG. 10A, and a strong correlation of DPS Platform WASF1 data to wet Platform WASF1 data (R2=0.861) is observed FIG. 10B. FIG. 10A is a graph showing preliminary verification of the analytical comparability of wet vs. DPS for sampling for NFL and WASF1 measurements of de-identified, archived plasma specimens from full spectrum TBI subjects (1 pdi to 6 months) with the Platform high sensitivity immunoassay platform. FIG. 10B is a graph showing preliminary verification of the analytical comparability of wet vs. DPS for sampling for WASF1 measurements of de-identified, archived plasma specimens from full spectrum TBI subjects (1 pdi to 6 months) with the Platform high sensitivity immunoassay platform.

Third, preliminary verification of the of the analytical comparability of wet vs. DPS for sampling with respect to the recovery of TBI biomarkers is presented in FIG. 11A-11D. FIG. 11A is a graph showing preliminary verification of the analytical comparability of wet vs. DPS for sampling with respect to the recovery of TBI biomarkers with a serial dilution of recombinant protein biomarker GFAP spanning five orders of magnitude for a spiking recovery study in pooled plasma from 10 healthy controls age 40-45. FIG. 11B is a graph showing preliminary verification of the analytical comparability of wet vs. DPS for sampling with respect to the recovery of TBI biomarkers with a serial dilution of recombinant protein biomarker NFL spanning five orders of magnitude for a spiking recovery study in pooled plasma from 10 healthy controls age 40-45. FIG. 11C is a graph showing preliminary verification of the analytical comparability of wet vs. DPS for sampling with respect to the recovery of TBI biomarkers with a serial dilution of recombinant protein biomarker Tau spanning five orders of magnitude for a spiking recovery study in pooled plasma from 10 healthy controls age 40-45. FIG. 11D is a graph showing preliminary verification of the analytical comparability of wet vs. DPS for sampling with respect to the recovery of TBI biomarkers with a serial dilution of recombinant protein biomarker UCH-L1 spanning five orders of magnitude for a spiking recovery study in pooled plasma from 10 healthy controls age 40-45. For FIGS. 11A-11D, assays were performed with the third immunoassay platform (third) as a benchmark.

A wide dynamic range is an important analytical figure of merit for the inventive SPS. Likewise, the impact of matrix affects must be assessed and accounted for to maximize protein recovery from the DPS collection disk. Therefore, a spiking recovery study is performed in pooled plasma from 10 healthy controls age 40-45 with serial dilutions of four recombinant protein biomarkers (GFAP, NFL, Tau, and UCH-L1) spanning five orders of magnitude. Assays are performed with the third immunoassay platform (third) as a benchmark. Excellent recovery (>75%) is observed for all four biomarkers except for Tau at the lowest two levels, as shown in FIGS. 11A-11D. This provides evidence that the conditions for eluting proteins captured on the DPS collection disk have been optimized. For example, several mild surfactants, surfactant concentrations, buffer pH and osmolality, as well as extraction time and volume are investigated. The concentration-response relationship of biomarkers in the panel are carefully established with spiking recovery studies such as these as well as additional studies with pooled samples from mild, moderate, and severe TBI patients, as well as healthy controls.

Lastly, supporting evidence for the need for high frequency longitudinal sampling and measurement is presented in the Longitudinal Pilot Data for selected biomarkers from the inventive panel (GFAP, NFL, PNF-H, Tau, P-Tau, IL-6, and MOG) in wet serum specimens, as shown in FIGS. 3A-8. Notably, the distinct temporal profiles and peaks or waves of GFAP, Tau, pTau, IL-6, NFL, and MOG strongly argue for high frequency DPS sampling, from the acute to subacute/chronic phases, to help monitor the TBI patients in the military environment, to provide the best SPS possible for prehospital, hospital, and at home medical care, and to accelerate recovery from TBI.

### Example 4

Moderate-Severe Civilian Traumatic Brain Injury Study—30 subjects with moderate-severe TBI are studied for biomarker levels in various tissues and at various times following injury over the course of 12 months to demonstrate the safety and feasibility of the SPS platform. Each of these subjects is over age 18, has a moderate to severe TBI as defined by GCS less than or equal to 13 with positive evidence of TBI on CT imaging, and has a GCS motor score of less than 6. Additionally, 15 age and sex matched control subjects are likewise studied.

The data is collected in the form of a Case report form (CRF) which is mainly collected by clinical service staff if it is part of standard data collection or standard of care. Case report form (CRF) data is collected for each subject throughout their participation in the trial. These include data derived from chart review, as well as telephone interviews post-discharge. This data includes: subject age, sex, GCS, time/date of initial injury, polytrauma status, ICU/Floor status, and use of mechanical ventilation.

TBI phenotype on CT is categorized, (e.g., epidural hematoma, diffuse axonal injury, subdural hematoma, intraparenchymal hemorrhage, cerebral edema, subarachnoid hemorrhage, largest intracranial lesion, and herniation), and evolution of CT findings during hospitalization is collected.

Clinical course including operative interventions, intracranial pressure monitoring, PbtO2 monitoring, and the development of adverse events are collected.

Clinical outcome measures are collected to quantify severity of injury and include: worsening of neurologic exam, need for operative intervention, need for intubation, duration of hospital stay/ICU care, 30-day and 1-year mortality/readmission rates and Glasgow extended and Disability rating Scale (DRS) at 1 and 6 month are collected.

All data collected is HIPAA-compliant.

In hospital: Serial wet plasma specimens and DPS specimens are collected from in-patients at the following time points: enrollment (with post-injury time recorded), 24 h, and twice daily prior to hospital discharge for up to 14 days (28 time points), once at 30 days, and once at discharge (n=30 each of wet and dried specimens total up to discharge).

A single wet plasma specimen and DPS specimen is collected from the healthy controls.

Post-discharge: For all TBI patients, prior to discharge, the patient himself/herself and/or a caregiving family member or custodian is trained how to use the CP and will be provided with 8 DPS collection kits (4 to use, 4 as back-up). They are instructed to collect 4 more sets of DPS specimens (at 7-, 14-, 30-, and 60-days post-discharge). Samples are mailed back by pre-paid courier to the study's local laboratory, without refrigeration. At the laboratory, the QR code of each CP is scanned to uniquely identify the TBI out-patient specimen for analysis.

Clinical course including operative interventions, intracranial pressure monitoring, PbtO2 monitoring, and the development of adverse events are collected. Clinical outcome measures are also collected to quantify severity of injury and patient outcome and include: worsening of neurologic exam, need for operative intervention, need for intubation, duration of hospital stay/ICU care, 30-day and 1-year mortality/readmission rates and Glasgow outcome scale extended and Disability rating Scale (DRS) at 1 and 6 months.

The Moderate-Severe Civilian Traumatic Brain Injury Study demonstrates that:
the SPS (Single platform Solution for TBI temporal biomarkers panel) can be deployed within a health care setting after acute moderate-severe TBI to collect serial blood samples, arrange for cold-chain shipment to single test site for TBI temporal biomarker panel analysis.
the SPS platform can be deployed for use by patients and their caregivers after discharge for incident TBI, to collect serial dry blood spot samples and to arrange to ambient temperature shipment to single test site for TBI temporal biomarker panel analyses.
at the hospital setting the acutely collected dry plasma sample and its wet plasma sample counterpart from TBI and control subjects yield key biomarker levels with robust correlation r≥0.60)
the SPS platform can be employed to distinguish wet and dry plasma samples from TBI subjects from age-matched control subjects.
The SPS platform can be deployed within a health care setting after acute moderate-severe TBI to collect serial blood samples and return for shipment and analyses.
The SPS platform can be deployed for use by patients and their caregivers after discharge for incident TBI, to collect serial blood samples and return for shipment and analyses.
The SPS platform will be piloted in age-matched control subjects

### Example 5

200 severe TBI (sTBI) subjects are enrolled in the clinical trial. Of the patients enrolled, 114 subjects consented to donate their biosamples remaining after completion of the trial for future studies, where ˜80 of these patients have sufficient biosamples remaining to be included in the current project. The following characteristics for these patients are typical of a sTBI population. Average age of the patients is 32.5 years. 18 (16%) are women, and 96 (84%) are men. 23 (20%) are black, 3 (3%) are Asian, 27 (24%) are white non-Hispanic, and 61 (54%) are white Hispanic. Marshall CT score: Diffuse injury I (0), Diffuse injury I (49), Diffuse injury III (30), Diffuse injury IV (0), Mass Lesion-Evacuated (32), Mass Lesion—non-evacuated (3). Six mo. outcome (GOSE) results are: Good recovery (15); Moderate Disability (29); Severe Disability (42), Vegetative (6), Dead (15), Lost to follow-up (7). Other data collected include Prehospital hypotension, Prehospital hypoxia, Sepsis-related Organ Failure Assessment (SOFA) Score, Abbreviated Injury Scale (AIS), Injury Severity Score (ISS) and Acute Physiology and Chronic Health Evaluation I (APACHE II). Whenever possible, serial serum and CSF were collected at 6, 12, 18, 24, 48, 72, 96, 120, 144, 168, 192, 216, 240 hrs post-injury (Aisiku, Yamal et al. 2016).

New enrollment Cohorts (BCM) Obtain IRB approval of new subject enrollment and initiate new enrollment at BCM of: (a) sTBI subjects (N=63): inclusion criteria are blunt severe TBI with motor GCS≤5, age >=18, enrolled within 12 hr of injury; exclusion criteria are penetrating injury, life-threatening systemic injuries, spinal cord injury, severe pre-existing disease that might interfere with follow-up for 6 months. (b) Orthopedic injury controls (N=20): inclusion criteria are extremity sprain or fracture but no TBI, enrolled within 12 hr of injury, age and gender matched to TBI subjects; exclusion criteria are pre-existing health problems, TBI within previous year (c) Healthy controls (N=20): inclusion criteria are normal volunteers with no history of TBI within past year, age and gender matched to sTBI subjects; exclusion criteria are pre-existing health problems, TBI within previous year. For TBI subjects, collect serial CSF and serum samples (8 mL blood collection tube) at post-injury time 12 h, daily samples (1 to 10 dpi); and expanded serum samples at 14 days, 1, 3, and 6 mo.; collect Glasgow outcome Scale-extended (GOSE) and Disability Rating Scale (DRS) at 1,3, and 6 mo; and collect CT lesion volume changes on admission, at 24 h, and again based on clinical measures. For HC subjects, single time point serum samples are collected.

Biosample collection, processing and cold-chain transport and storage. Timed Serum (using 8 mL blood collection tubes with clot separator) and CSF sample collection (into 15 mL conical tubes disposable centrifuge tubes (BD) followed our pre-established standard operations procedures, which are guided by published Biospecimens and Biomarkers Recommendations from the TBI common Data Element Working Group (Manley, Diaz-Arrastia et al. 2010). Each serum samples is about 3-4 mL, and 10 mL CSF. All samples are stored as 500 μL micro-aliquots at −85° C. freezer until use.

### Example 6

Biostatistically, the two primary comparisons based on the clinical study objectives and study design include comparing the feasibility and utility of dry plasma collection vs, their traditional wet plasma counterpart in terms of their utility in report key TBI biomarker levels on the single assay platform and comparing key biomarker levels (we or dry plasma) in severe-moderate TBI subjects vs. control subjects.

For dry vs, wet plasma correlation, based on the data on several biomarkers (NFL, GFAP, WASF1 and VAMP5, UCH-L1, the R value ranges from 0.65 to 0.95. Thus, (α) is set at 0.05 (2-tailed) and (1−β) at 0.80 and R=0.50 (as a conservative projection), and a minimum sample size N=29 is calculated. Since the experimental group includes N=30 TBI and N=15 normal controls for all key biomarker measurement—(total N=45)—is sufficiently powerful to examine wet-dry plasm biomarker level correlation.

For TBI vs. healthy control comparison, a power analysis is performed based on pilot data to detect a difference in the means or median of each biomarker between the two groups (TBI vs. control), with (α) at 0.05 (2-tailed) and (1−β) at 0.80. based on the pilot data with key protein biomarkers for NFL as example, a mean group fold difference of 1.50 and standard deviation of 28% and obtained Cohen's d=1.307 are obtained. Requiring a minimum of 11 subjects per group. For another marker WASF1, which has a smaller differences of mean values (TBI vs. control), a mean group difference of 1.30 with 27% standard deviation is used, which yields a Cohen's d=0.959 and calculated sample size of N=15 per group Thus, the enrolled 30 TBI subjects and 15 healthy controls is sufficiently powered.

Data Analysis Plan: (I) Descriptive analysis Prior to conducting the analyses, the statistical properties of our biomarkers, outcome measures, and other demographic and clinical characteristics are evaluated. Descriptive statistics, (means, medians, other percentiles) and dispersion (standard deviations, ranges) are computed for continuous data. Outliers, normality, and missing data are checked for. Frequency distributions are calculated for categorical data. Repeated measures data are “binned” over discrete time intervals.

(II) line graph for each biomarker (or ratio between two markers) are created to inspect the temporal trends for each of these biomarkers, stratified by groups. To compare a given biomarker at each time point between the two outcome groups, a two-sample t-test or Wilcoxon rank-sum test is used depending on the distribution of the biomarker.

(III) both median comparison between groups (TBI and controls) and receiver operating characteristics) ROC analysis are conducted for each biomarker measured at their best time point, and for each binary outcome of interest as noted above (e.g., TBI vs. controls), to investigate the sensitivity and specificity of each marker to predict TBI. The difference between the area under the two ROC curves are tested (one for one biomarker and one for another) using the Hanley and McNeil (1983) method 29.

(III) For wet to dry plasma comparison for each of the key biomarkers, all subjects (TBI, controls) or TBI subjects alone and plot correlation graphs (X, Y) are examined. Each data point represents a single subject, with X, and Y as values of a biomarker measured in wet vs. dry plasma from the same time point. Linear regression will then examine the Pearson's r (correlation coefficient) and R square, if the r value is different from zero. With a p value of significance threshold set at 0.05.

(III) For exploratory analysis, the addition of biomarkers are examined to determine it they will improve prediction of outcome measures. Multivariable logistic regression models are generated using important clinical factors (e.g., injury severity, age, sex) and single biomarker values or TRAJ group membership for each significant bivariate biomarker association with an outcome. The best time point of each biomarker is chosen that has the greatest predictive ability based on previous analyses. Then two multivariable models are built, with/without that biomarker value measured at its best time point, to predict the outcome. The independent association of the IMPACT score is assessed by the inclusion of both in the same model (REF). Multivariable models with random effects for longitudinal data are considered.

(VI) Again, as exploratory analysis, Generalized Linear Mixed Models and Trajectory analysis are employed: When dealing with repeated measures from the same patients over time, random effects are added in the model to allow for clustering effect within each patient using mixed-effect modeling 30. Similarly, trajectory class analysis is used to examine if different outcome groupings might have different temporal profiles or trajectories.

Power Analysis: The power analysis for the R33 phase is based on testing the AUC of the ROC curve to be above a minimum of 80%. Based on preliminary data, approximately 41% of patients show a favorable GOSE outcome. A sample size of N=63 sTBI patients results in at least 80% power to detect an AUC effect size from 80% to 95%. Power is increased for comparisons between sTBI and controls. Additionally, power analysis is performed based on pilot data to detect a difference in the means of each biomarker between the two sTBI and control groups, with (α) at 0.05 (2-tailed) and (1−β) at 0.80 based on our pilot data, as shown in FIG. 4C, it is found that the two main outcome groups (GOSE≤4 and ≥5) have a mean group fold difference of 1.35 and standard deviation of 36% and obtained Cohen's d=−0.814, and requiring a minimum 20 subjects per group. Thus, it is concluded that least n=63 TBI subjects in both R61/R33 are sufficiently powered. Similarly, for control vs. sTBI, a group difference of 1.5 and 30% deviation are found based on the pilot data, as shown in FIGS. 2-3D and obtained Cohen's d=1.307 and minimal N=11 per group. Thus, the R33 N=63 TBI and N=40 controls are sufficiently powered. The study is not powered for detection in subgroups/subphenotypes (e.g., sex), so these are considered as exploratory and interpreted with caution. To account for 5% loss to follow-up for 6-month outcome, 63 sTBI patients are recruited. Prior to conducting the analyses, the statistical properties of our biomarkers, outcome measures, and other demographic and clinical characteristics are evaluated. Descriptive statistics, (means, medians, other percentiles) and dispersion (standard deviations, ranges) are computed for continuous data. Outliers, normality, and missing data are checked for. Frequency distributions are calculated for categorical data. Repeated measures data are “binned” over discrete time intervals (e.g., days: acute markers; months: chronic markers) prior to analyses. First, a line graph for each miRNA (or ratio between two markers) is created to inspect the temporal trends for each of these biomarkers, stratified by groups. To compare a given biomarker at each time point between the two outcome groups, two-sample t-test or Wilcoxon rank sum test is utilized depending on the distribution of the biomarker. Second, multivariable logistic regression models are generated using important clinical factors (e.g., injury severity, age, sex) and single miRNA values or TRAJ group membership for each significant bivariate miRNA association with an outcome. The best time point of each miRNA is chosen that has the greatest predictive ability based on previous analyses. Then two multivariable models are built, with/without that miRNA measured at its best time point, to predict the outcome. The independent association of the IMPACT score is assessed by inclusion of both in the same model and estimate AUC with and without the IMPACT score. Multivariable models with random effects for longitudinal data are considered. Third, ROC analysis for each biomarker measured are conducted at their best time point, and for each binary outcome of interest as noted above (e.g., GOSE, DRS scores, CT lesion volume increase), to investigate the AUC of each marker to predict the outcome. The difference between the area under the two ROC curves are tested (one for the model with the biomarker of interest and one without) using the Hanley and McNeil (1983) method²⁹. Fourth, when dealing with repeated measures from the same patients over time, random effects are added in the model to allow for clustering effect within each patient using mixed effect modeling³⁰. Similarly, trajectory class analysis^{31,32} are used to examine if different outcome groupings might have different temporal profile or trajectory.

Methods involving conventional biological techniques are described herein. Such techniques are generally known in the art and are described in detail in methodology treatises such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Immunological methods (e.g., preparation of antigen-specific antibodies, immunoprecipitation, and immunoblotting) are described, e.g., in Current Protocols in Immunology, ed. Coligan et al., John Wiley & Sons, New York, 1991; and Methods of Immunological Analysis, ed. Masseyeff et al., John Wiley & Sons, New York, 1992. The entire contents of each of the aforementioned publications are incorporated herein by reference as if each were explicitly included herein in their entirety.

### Example 7

Table 4 below shows Examples of proteomic-based discovery of panel of TBI serum and plasma based biomarker proteins elevated in TBI with very strong diagnostic and temporal biomarker properties. These candidates are identified based on (A) comparing the average (ave) TBI D1 serum levels, D14 levels and 6 mo. serum levels (N=7 each) to average healthy control serum levels (N=7), expressed as a ratio, respectively), (B) Comparing pooled severe TBI D1 (pooled evenly from N=10), two separately pooled moderate-mild TBI plasma levels designated as #1 and #2 (pooled evenly from N=12 each) to pooled healthy control plasma (pooled evenly from 200 subjects) levels as a ratio. All the short-listed biomarker candidates shown in this table have both one average TBI/ave control serum ratio and one pooled TBI/pooled control plasma ratio of at least 1.40 (i.e., 1.40 fold increase) in at least one time interval. Those that reach such criterion are indicated with (*). Protein abbreviated name, protein full name and Uniprot accession no are shown. Under the “Brain injury biomarker clinical utility potentials” column, those with the highest, high, moderately high overall differential TBI/control ratio are indicated with ++++, +++, ++ and +, respectively. n.a.—data not available.

TABLE 4

| | | | | | | Performance in Plasma | | | |
|---|---|---|---|---|---|---|---|---|---|
| | | | Performance in Serum | | | | Pooled | Pooled | |
| | | | | | Ave. | Pooled | Moderate- | Moderate- | Brain |
| | | | Ave. | Ave. TBI | TBI | Severe | mild TBI | mild TBI | injury |
| Brain injury protein biomarker candidates | | | TBI | D14/ | 6 mo./ | TBI D1/ | D1 #1/ | D1 #2/ | biomarker |
| Protein | Uniprot | | D1/ave. | Ave. | Ave. | pooled | Pooled | Pooled | clinical |
| biomarker | Accession | Full Protein | Control | Controls | Control | Control | Controls | Controls | utility |
| Name (assay) | No. | name | Ratio | Ratio | Ratio | Ratio | Ratio | Ratio | potential |
| GFAP | P14136 | Glial fibrillary | 19.92 (*) | 0.78 | 1.65 (*) | 264.56 (*) | 11.77 (*) | n.a. | ++++ |
| (SIMOA) | | acidic protein | | | | | | | |
| NF-H (Ella ) | P12036 | Neurofilament- | 0.304 | 2.083 (*) | 1.121 | 4.457 (*) | 1.755 (*) | 1.659 (*) | ++++ |
| | | protein-Heavy | | | | | | | |
| NF-L (Ella ) | P07196 | Neurofilament- | 0.709 | 1.901 (*) | 1.226 | 4.128 (*) | 1.350 | 1.528 (*) | ++++ |
| | | protein-Light | | | | | | | |
| NFL | P07196 | Neurofilament- | 1.19 | 2.84 (*) | 4.36 (*) | 6.51 (*) | 2.07 (*) | n.a. | ++++ |
| (SIMOA) | | protein-Light | | | | | | | |
| UCH-L1 | P09936 | Ubiquitin C- | 2.42 (*) | 1.36 | 1.38 | 26.19 (*) | 6.01 (*) | n.a. | ++++ |
| (SIMOA) | | terminal | | | | | | | |
| | | hydrolase- L1 | | | | | | | |
| MAPT/TAU | P10636 | MAPT tau protein | 1.234 | 1.270 | 1.240 | 2.449 (*) | 0.939 | 1.949 (*) | +++ |
| NF-L | P07196 | Neurofilament- | 1.1 | 1.4 (*) | 0.99 | 1.532 (*) | 1.156 | 1.126 | +++ |
| | | protein-Light | | | | | | | |
| CAMKK1 | Q8N5S9 | Calcium/ | 0.940 | 1.543 (*) | 1.234 | 1.408 (*) | 0.720 | 1.448 (*) | +++ |
| | | Calmodulin | | | | | | | |
| | | Dependent | | | | | | | |
| | | Protein kinase | | | | | | | |
| | | kinase 1 | | | | | | | |
| DCTN2 | Q13561 | Dinectin-2 | 1.636 (*) | 3.333 (*) | 2.317 (*) | 9.699 (*) | 10.486 (*) | 13.424 (*) | +++ |
| DUSP3 | P51452 | DUSP3 (Dual | 0.852 | 2.468 (*) | 1.535 (*) | 1.414 (*) | 2.670 (*) | 2.060 (*) | +++ |
| | | Specificity | | | | | | | |
| | | Phosphatase 3) | | | | | | | |
| PRDX6 | P30041 | Peroxiredoxin 6 | 0.880 | 1.317 | 1.119 | 2.592 (*) | 2.667 (*) | 2.879 (*) | +++ |
| VAMP5 | 095183 | Vesicle Associated | 0.580 | 1.882 (*) | 1.351 | 5.101 (*) | 1.592 (*) | 3.437 (*) | +++ |
| | | Membrane | | | | | | | |
| | | Protein 5 | | | | | | | |
| WASF1 | Q92558 | WASP Family | 0.735 | 1.693 (*) | 1.450 (*) | 4.522 (*) | 3.904 (*) | 4.202 (*) | +++ |
| | | Member 1 | | | | | | | |
| WASF3 | Q9UPY6 | WASP Family | 0.851 | 1.691 (*) | 1.458 (*) | 4.983 (*) | 4.570 (*) | 5.281 (*) | +++ |
| | | Member 3 | | | | | | | |
| BCR | P11274 | Breakpoint cluster | 0.671 | 1.819 (*) | 1.637 (*) | 2.873 (*) | 1.748 (*) | 2.554 (*) | ++ |
| | | region protein | | | | | | | |
| COL4A3BP, | Q9Y5P4 | Ceramide transfer | 0.964 | 1.569 (*) | 1.417 (*) | 1.613 (*) | 1.738 (*) | 1.710 (*) | ++ |
| CERT1 | | protein (CERT1) | | | | | | | |
| FHIT | P49789 | FHIT (Fragile | 0.730 | 1.455 | 1.386 | 2.327 (*) | 2.639 (*) | 2.616 (*) | ++ |
| | | Histidine Triad | | | | | | | |
| | | Diadenosine | | | | | | | |
| | | Triphosphatase) | | | | | | | |
| IMPA1 | P29218 | IMPA1 (Inositol | 0.914 | 1.598 (*) | 1.337 | 1.696 (*) | 1.798 (*) | 1.792 (*) | ++ |
| | | Monophosphatase | | | | | | | |
| | | 1) | | | | | | | |
| ING1 | Q9UK53 | ING1 | 1.076 | 1.941 (*) | 1.663 (*) | 1.53 (*) | 1.20 | 1.74 (*) | ++ |
| | | (Inhibitor Of | | | | | | | |
| | | Growth Family | | | | | | | |
| | | Member 1) | | | | | | | |
| MAP2K6 | P52564 | Dual specificity | 0.828 | 1.429 (*) | 1.347 | 2.021 (*) | 1.954 (*) | 2.294 (*) | ++ |
| | | mitogen-activated | | | | | | | |
| | | protein kinase | | | | | | | |
| | | kinase 6 | | | | | | | |
| METAP1D | Q6UB28 | METAP1D | 0.815 | 1.240 | 1.235 | 2.435 (*) | 1.599 (*) | 2.040 (*) | ++ |
| | | (Methionyl | | | | | | | |
| | | Aminopeptidase | | | | | | | |
| | | Type 1D) | | | | | | | |
| NAA10 | P41227 | N-alpha- | 0.778 | 1.320 | 1.232 | 1.574 (*) | 1.681 (*) | 1.750 (*) | ++ |
| | | acetyltransferase | | | | | | | |
| | | 10 | | | | | | | |
| NFATC1 | 095644 | N-Alpha- | 0.773 | 2.792 (*) | 1.976 (*) | 3.611 (*) | 3.095 (*) | 3.262 (*) | ++ |
| | | Acetyltransferase 1 | | | | | | | |
| SULT2A1 | Q06520 | Sulfotransferase | 0.924 | 1.447 (*) | 0.761 | 2.040 (*) | 3.356 (*) | 2.978 (*) | ++ |
| | | 2A1 | | | | | | | |
| ZBTB16 | Q05516 | Zinc finger | 0.739 | 2.059 (*) | 1.448 (*) | 6.972 (*) | 5.473 (*) | 6.603 (*) | ++ |
| | | and BTB | | | | | | | |
| | | domain-containing | | | | | | | |
| | | protein 16 | | | | | | | |
| ARHGEF12 | Q9NZN5 | Rho Guanine | 0.536 | 1.487 (*) | 1.439 (*) | 2.534 (*) | 1.707 (*) | 2.296 (*) | ++ |
| | | Nucleotide | | | | | | | |
| | | Exchange | | | | | | | |
| | | Factor 12 | | | | | | | |
| TACC3 | Q9Y6A5 | Transforming | 0.642 | 1.663 (*) | 1.428 (*) | 3.305 (*) | 3.272 (*) | 3.419 (*) | ++ |
| | | acidic coiled-coil- | | | | | | | |
| | | containing | | | | | | | |
| | | protein 3 | | | | | | | |

### Example 8

Table 5 shows examples of proteomic-based discovery of a panel of TBI serum and plasma based biomarker proteins elevated in TBI with strong diagnostics and temporal biomarker properties. These candidates were identified based on (A) comparing the average (ave). TBI D1 serum levels, D14 levels and 6 mo. serum levels (N=7 each) to average healthy control serum levels (N=7), expressed as a ratio, respectively), (B) Comparing pooled severe TBI D1 (pooled evenly from N=10), two separately pooled moderate-mild TBI plasma levels designated as #1 and #2 (pooled evenly from N=12 each) to pooled healthy control plasma (pooled evenly from 200 subjects) levels as a ratio. All the short-listed biomarker candidates shown in this table have one ave TBI/ave control serum ratio or one pooled TBI/pooled control plasma ratio of at least 1.40 (i.e. 1.40 fold increase) in at least one time interval. Those that reach such criterion are indicated with (*). Protein abbreviated name, protein full name and Uniprot accession no. Under the “Brain injury biomarker clinical utility potentials” column, all markers are rated as having moderate high (if it has with brain or disease mechanism relevance) (indicated with ++) or moderate overall differential TBI/control ratio (indicated with “+” respectively.

TABLE 5

| | | | | | | Performance in Plasma | | | |
|---|---|---|---|---|---|---|---|---|---|
| | | | Performance in Serum | | | | Pooled | Pooled | |
| | | | Ave. | Ave. TBI | Ave. TBI | Pooled | Moderate- | Moderate- | Brain |
| Brain injury protein biomarker candidates | | | TBI | D14/ | 6 mo./ | Severe TBI | mild TBI D1 | mild TBI D1 | injury |
| Protein | | | D1/ave. | Ave. | Ave. | D1/pooled | #1/Pooled | #2/Pooled | biomarker |
| biomarker | Uniprot | | Control | Controls | Control | Control | Controls | Controls | clinical |
| Name | Accession | Full Protein | Serum | Serum | Serum | Plasma | Plasma | Plasma | utility |
| (assay) | No. | name | Ratio | Ratio | Ratio | Ratio | Ratio | Ratio | potential |
| AARSD1 | Q9BTE6 | Alanyl-tRNA | 0.718 | 1.182 | 1.162 | 1.91 (*) | 1.58 (*) | 2.31 (*) | + |
| | | editing protein | | | | | | | |
| | | Aarsd1 | | | | | | | |
| ABHD14B | Q96IU4 | Protein | 1.022 | 1.494 (*) | 1.245 | 1.82 (*) | 2.03 (*) | 2.01 (*) | + |
| | | ABHD14B | | | | | | | |
| CBL | P22681 | E3 ubiquitin- | 0.820 | 1.655 (*) | 1.421 (*) | 1.27 | 0.95 | 1.15 | + |
| | | protein ligase | | | | | | | |
| CLSTN3 | Q9BQT | Calsyntenin-3 | 0.909 | 1.142 | 1.051 | 1.48 (*) | 2.02 (*) | 1.57 (*) | + |
| | 9 | | | | | | | | |
| FKBP5 | Q13451 | Peptidyl-prolyl | 0.902 | 1.304 | 1.241 | 1.96 (*) | 2.07 (*) | 2.11 (*) | + |
| | | cis-trans | | | | | | | |
| | | isomerase-5 | | | | | | | |
| | | FKBP5 | | | | | | | |
| FKBP7 | Q9Y680 | Peptidyl-prolyl | 0.788 | 1.209 | 1.049 | 1.57 (*) | 1.30 | 1.61 (*) | + |
| | | cis-trans | | | | | | | |
| | | isomerase-7 | | | | | | | |
| G-CSF | P09919 | Granulocyte | 1.153 | 1.110 | 0.949 | 1.81 (*) | 1.21 | 1.41 (*) | + |
| | | colony- | | | | | | | |
| | | stimulating | | | | | | | |
| | | factor (CSF3) | | | | | | | |
| HAGH | Q16775 | Hydroxyacylglut | 0.805 | 0.919 | 1.055 | 1.41 (*) | 1.32 | 1.51 (*) | + |
| | | athione | | | | | | | |
| | | hydrolase, | | | | | | | |
| | | mitochondrial | | | | | | | |
| HMOX2 | P30519 | Heme | 0.997 | 1.403 (*) | 1.320 | 1.33 | 1.07 | 1.19 | + |
| | | oxygenase 2 | | | | | | | |
| HSP90B1 | P14625 | Endoplasmin | 1.212 | 1.188 | 1.247 | 1.23 | 3.91 (*) | 1.52 (*) | + |
| KIF1BP | Q96EK5 | KIF-binding | 0.852 | 1.238 | 1.164 | 1.89 (*) | 2.00 (*) | 2.00 (*) | + |
| | | protein | | | | | | | |
| LAT | 043561 | Linker for | 0.846 | 1.018 | 1.060 | 1.40 (*) | 1.40 (*) | 1.43 (*) | + |
| | | activation of T- | | | | | | | |
| | | cells family | | | | | | | |
| | | member 1 | | | | | | | |
| LXN | Q9BS40 | Latexin | 1.027 | 1.229 | 1.164 | 1.76 (*) | 2.01 (*) | 2.06 (*) | + |
| MAD1L1 | Q9Y6D9 | Mitotic spindle | 1.193 | 1.444 (*) | 1.291 | 1.37 | 1.36 | 1.64 (*) | + |
| | | assembly | | | | | | | |
| | | checkpoint | | | | | | | |
| | | protein MAD1 | | | | | | | |
| METAP1D | Q6UB2 | METAP1D | 0.815 | 1.240 | 1.235 | 2.435 (*) | 1.599 (*) | 2.040 (*) | + |
| | 8 | (Methionyl | | | | | | | |
| | | Aminopeptidase | | | | | | | |
| | | Type 1D) | | | | | | | |
| NINJ1 | Q92982 | Ninjurin-1 | 1.037 | 1.129 | 1.023 | 1.52 (*) | 0.90 | 1.17 | ++ |
| PFKM | P08237 | ATP-dependent | 0.869 | 1.345 | 1.181 | 2.52 (*) | 2.49 (*) | 2.91 (*) | + |
| | | 6-phospho- | | | | | | | |
| | | fructokinase, | | | | | | | |
| | | muscle type | | | | | | | |
| PLXNB1 | 043157 | Plexin-B1 | 0.775 | 0.753 | 0.575 | 2.15 (*) | 3.84 (*) | 3.18 (*) | + |
| PRTFDC1 | Q9NRG | Phosphoribosyl | 0.623 | 1.255 | 1.247 | 1.63 (*) | 1.74 (*) | 1.76 (*) | + |
| | 1 | transferase | | | | | | | |
| | | domain- | | | | | | | |
| | | containing | | | | | | | |
| | | protein 1 | | | | | | | |
| TBCB | Q99426 | Tubulin folding | 0.760 | 1.218 | 1.236 | 1.261 | 1.366 | 1.401 (*) | ++ |
| | | cofactor B | | | | | | | |
| ZBTB17 | Q13105 | Zinc finger and | 0.977 | 1.116 | 1.098 | 2.01 (*) | 1.37 | 1.57 (*) | + |
| | | BTB domain- | | | | | | | |
| | | containing | | | | | | | |
| | | protein 17 | | | | | | | |

### Example 9

Taking the above stated findings on unobvious and unique temporal profile of MOG, MOG-Ab, pTau, Tau WASF1 or WASF3, VAMP5 or VAMP2, CAMKKI, VEGF-A, MBP, IL-6 and other biomarkers demonstrated in this document, it argues for two important brain injury biomarker applications in relation to clinical patient care, management and monitoring (i) First, instead of measuring a single biomarker, it is clinically important to conduct simultaneous measurement of a panel of multiple biomarkers, preferably at least three markers, such as MOG plus two or more other biomarker proteins such as MOG-Ab, pTau, Tau, WASF1 or WASF3, VAMP5 or VAMP2, CAMKKI, VEGF-A, MBP, and IL-6, but not limiting to them. (ii) Second, it is of importance for clinical monitoring and care purposes to measure such panel of brain injury biomarkers at mre than one or multiple post-injury or post-disorder initiation time points repeatedly.

### Example 10

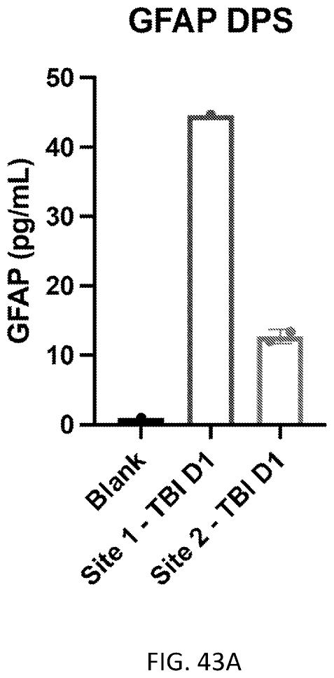

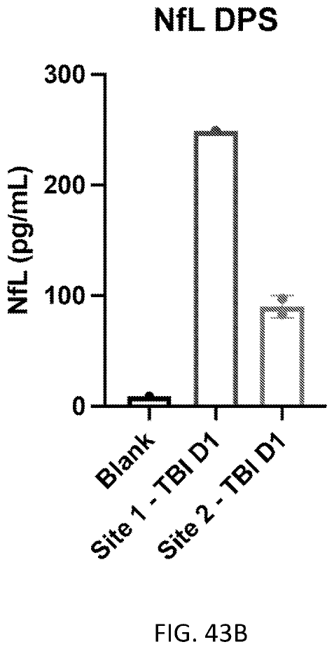

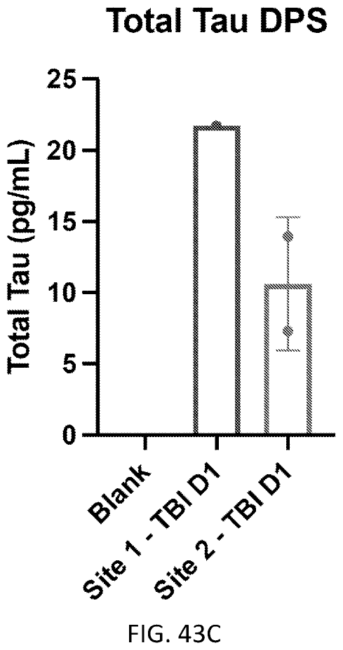

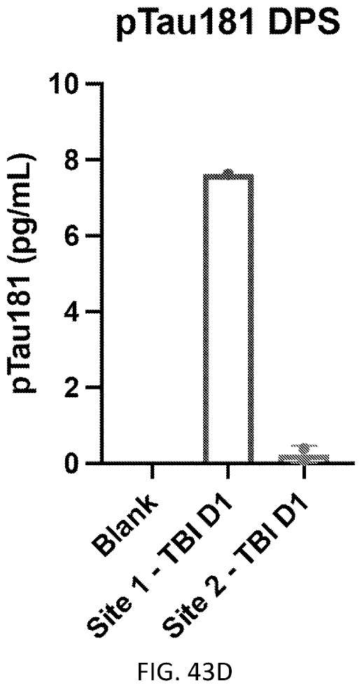

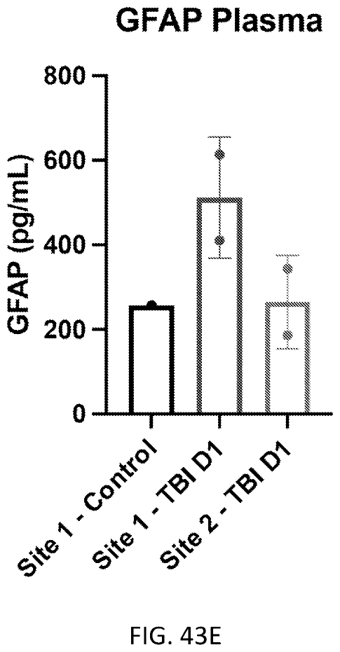

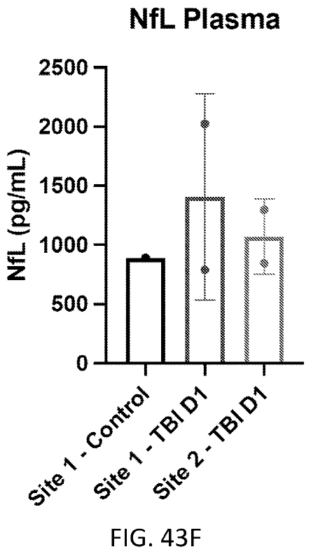

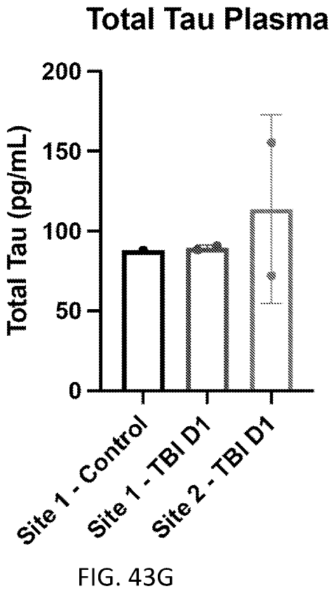

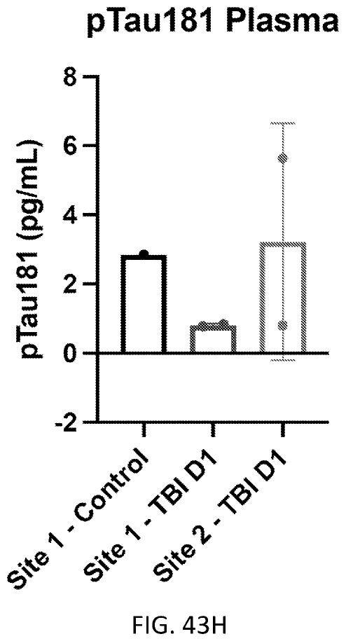

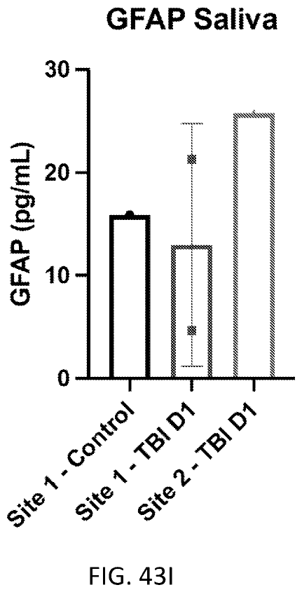

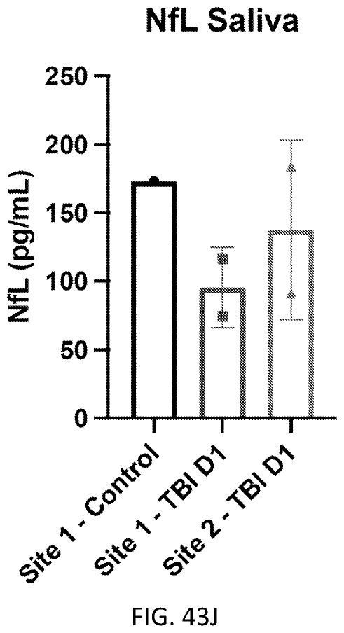

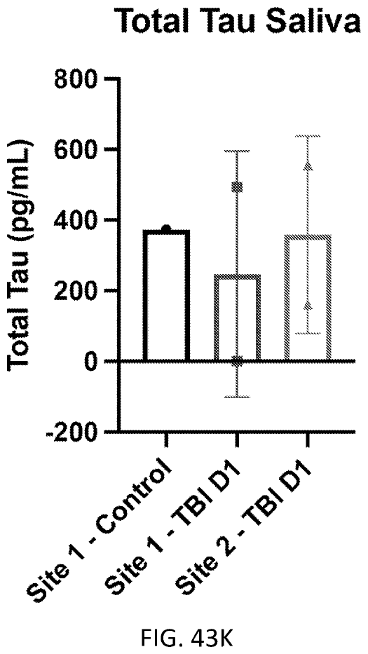

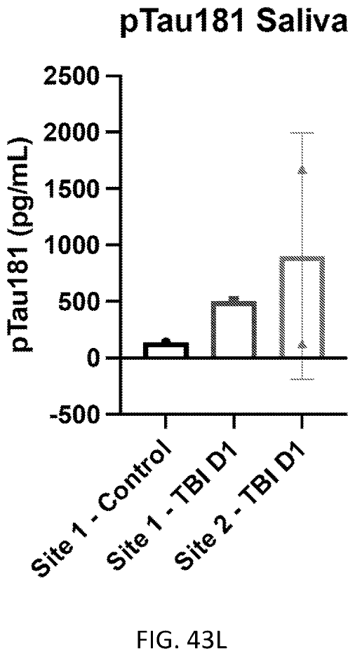

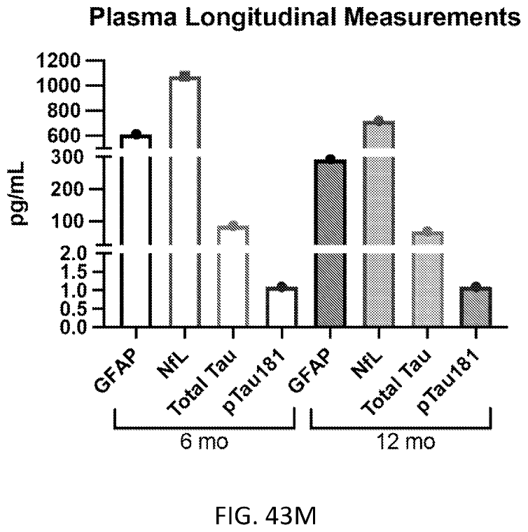

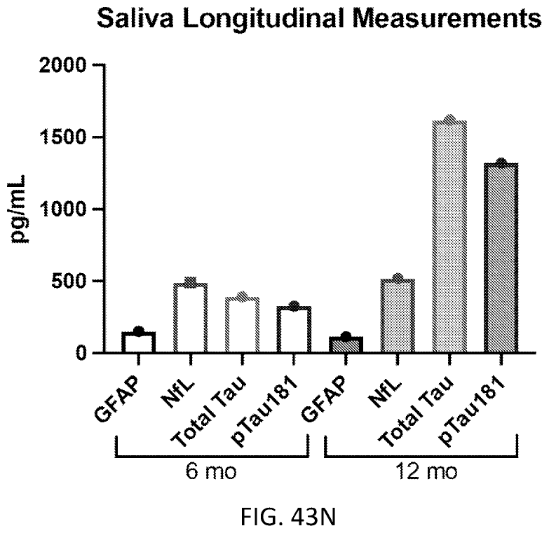

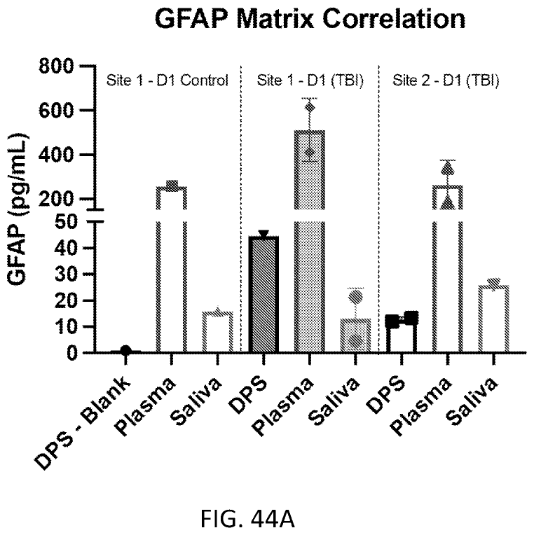

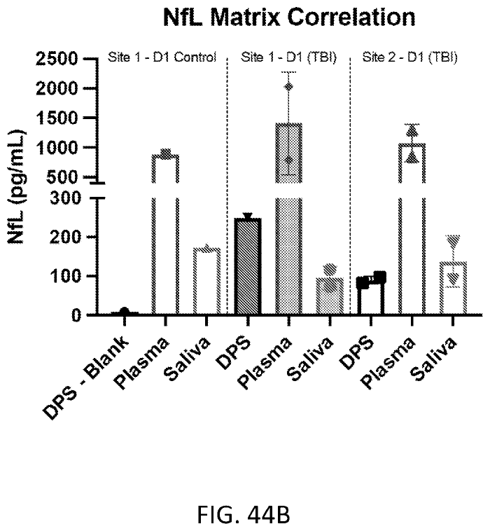

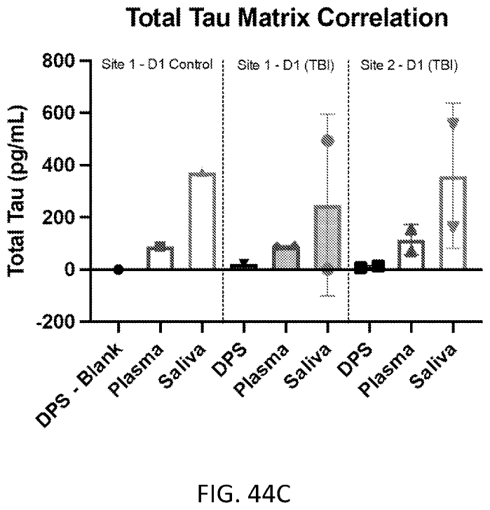

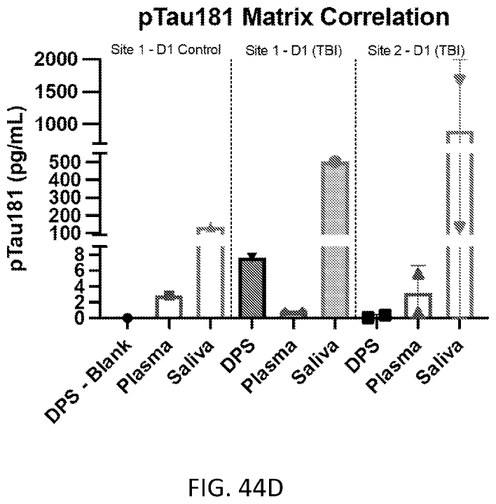

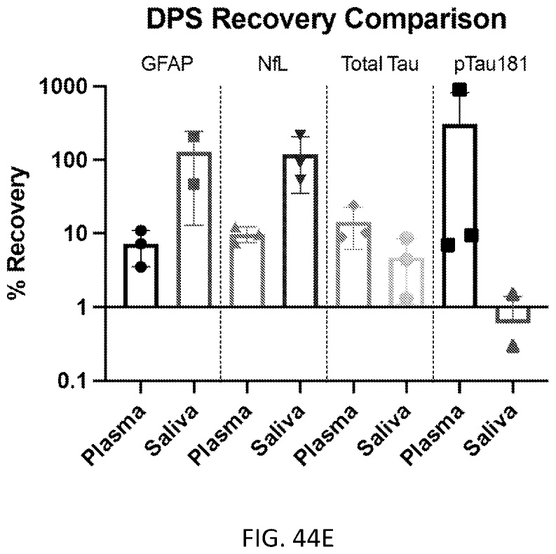

Patients presenting with a traumatic brain injury (TBI) are enrolled for biofluid sampling. Plasma, serum, saliva, and dried plasma spot (DPS) samples are collected from each patient. FIGS. 43A-44E show preliminary results from the first patients. For DPS samples, fingerprick capillary blood from each patient is collected by lancing the tip of the finger and allowing 3-4 hanging drops of blood to enter the Gryphon DPS device. The DPS device separates the red blood cells from the plasma through a capillary mechanism, where the plasma is then caught on a collection disc on the device and dried at ambient room temperature. Saliva is collected through a cotton swab syringe sampling device. The cotton swab is attached to the plunger of the syringe which is inserted under the tongue of the patient for a few minutes as the swab fills with saliva. The cotton swab and plunger are inserted into the barrel of the syringe device and pressed through a filter at the end of the syringe and into an Eppendorf collection tube. Plasma and serum are collected through conventional mechanisms. The plasma from the collection discs was eluted and measured in the same manner as wet plasma, demonstrating a low-cost effective plasma collection method. FIGS. 43A-L show successful recovery of Glial fibrillary acidic protein (GFAP), Neurofilament light (NfL), Total Tau, and pTau181 with the inventive DPS device (FIGS. 43A-D), plasma (FIGS. 43E-H), and saliva (FIGS. 43 I-L) from the same Control and TBI patients measuring GFAP, NfL, Total Tau, and pTau181 were also measured to show correlation of measurements between matrices which can use a correction factor later for normalization. FIGS. 43M and 43N are graphs showing longitudinal plasma and saliva samples, respectively, from unique TBI patients collected at 6 months and 12 months for measurement. FIGS. 44A-E present side-by-side matrix comparisons of each biomarker to show that correction factors can be used to correlate biomarker levels between matrices. Also shown is a rough recovery comparison of biomarkers collected on DPS compared to plasma and saliva. Higher than 100% recovery values indicate biomarker levels in saliva were higher than plasma. Separate preclinical experiments have shown similar recovery and biomarker measurements in blood collected from mice loaded onto our DPS device. The results are reproducible with dried saliva.

### REFERENCES CITED

Aisiku, I. P., J.-M. Yamal, P. Doshi, J. S. Benoit, S. Gopinath, J. C. Goodman and C. S. Robertson (2016). “Plasma cytokines IL-6, IL-8, and IL-10 are associated with the development of acute respiratory distress syndrome in patients with severe traumatic brain injury.” Critical Care 20(1):288.
Brito, A., T. W. Costantini, A. E. Berndtson, A. Smith, J. J. Doucet and L. N. Godat (2019). “Readmissions After Acute Hospitalization for Traumatic Brain Injury.” J Surg Res 244:332-337.
Curtis, K. A., K. M. Ambrose, M. S. Kennedy and S. M. Owen (2014). “Evaluation of dried blood spots with a multiplex assay for measuring recent HIV-1 infection.” PLoS One 9(9):e107153.
Czeiter, E., K. Amrein, B. Y. Gravesteijn, F. Lecky, D. K. Menon, S. Mondello, V. F. J. Newcombe, S. Richter, E. W. Steyerberg, T. V. Vyvere, J. Verheyden, H. Xu, Z. Yang, A. I. R. Maas, K. K. W. Wang, A. Buki, C.-T. Participants and Investigators (2020). “Blood biomarkers on admission in acute traumatic brain injury: Relations to severity, CT findings and care path in the CENTER-TBI study.” EBioMedicine 56:102785.
Galazka, G., M. P. Mycko, I. Selmaj, C. S. Raine and K. W. Selmaj (2018). “Multiple sclerosis: Serum-derived exosomes express myelin proteins.” Mult Scler 24(4): 449-458.
Hu, Y. Y., S. S. He, X. C. Wang, Q. H. Duan, S. Khatoon, K. Iqbal, I. Grundke-Iqbal and J. Z. Wang (2002). “Elevated levels of phosphorylated neurofilament proteins in cerebrospinal fluid of Alzheimer disease patients.” Neurosci Lett 320(3): 156-160.

Manley, G. T., R. Diaz-Arrastia, M. Brophy, D. Engel, C. Goodman, K. Gwinn, T. D. Veenstra, G. Ling, A. K. Ottens, F. Tortella and R. L. Hayes (2010). “Common Data Elements for Traumatic Brain Injury: Recommendations From the Biospecimens and Biomarkers Working Group.” Archives of Physical Medicine and Rehabilitation 91(11): 1667-1672-1672.
Massaro, A. N., Y. W. Wu, T. K. Bammler, J. W. MacDonald, A. Mathur, T. Chang, D. Mayock, S. B. Mulkey, K. van Meurs, Z. Afsharinejad and S. E. Juul (2019). “Dried blood spot compared to plasma measurements of blood-based biomarkers of brain injury in neonatal encephalopathy.” Pediatr Res 85(5): 655-661.
McKee, A. C., R. C. Cantu, C. J. Nowinski, E. T. Hedley-Whyte, B. E. Gavett, A. E. Budson, V. E. Santini, H. S. Lee, C. A. Kubilus and R. A. Stern (2009). “Chronic traumatic encephalopathy in athletes: progressive tauopathy after repetitive head injury.” J Neuropathol Exp Neurol 68(7): 709-735.
Mielke, M. M., J. A. Aakre, A. Algeciras-Schimnich, N. K. Proctor, M. M. Machulda, U. Eichenlaub, D. S. Knopman, P. Vemuri, J. Graff-Radford, C. R. Jack, Jr., R. C. Petersen and J. L. Dage (2022). “Comparison of CSF phosphorylated tau 181 and 217 for cognitive decline.” Alzheimers Dement 18(4): 602-611.
Mielke, M. M., J. L. Dage, R. D. Frank, A. Algeciras-Schimnich, D. S. Knopman, V. J. Lowe, G. Bu, P. Vemuri, J. Graff-Radford, C. R. Jack, Jr. and R. C. Petersen (2022). “Performance of plasma phosphorylated tau 181 and 217 in the community.” Nat Med 28(7): 1398-1405.
Mussa, A., V. P. Ciuffreda, P. Sauro, V. Pagliardini, S. Pagliardini, D. Carli, J. M. Kalish, F. Fagioli, E. Pavanello and G. B. Ferrero (2019). “Longitudinal Monitoring of Alpha-Fetoprotein by Dried Blood Spot for Hepatoblastoma Screening in Beckwith(-)Wiedemann Syndrome.” Cancers (Basel) 11(1).
Neselius, S., H. Zetterberg, K. Blennow, J. Marcusson and H. Brisby (2013). “Increased CSF levels of phosphorylated neurofilament heavy protein following bout in amateur boxers.” PLoS One 8(11): e81249.
Omalu, B. I., R. L. Hamilton, M. I. Kamboh, S. T. DeKosky and J. Bailes (2010). “Chronic traumatic encephalopathy (CTE) in a National Football League Player: Case report and emerging medicolegal practice questions.” J Forensic Nurs 6(1): 40-46.
Orszag, P. R. and E. J. Emanuel (2010). “Health care reform and cost control.” N Engl J Med 363(7): 601-603.
Shahim, P., A. Politis, A. van der Merwe, B. Moore, Y. Y. Chou, D. L. Pham, J. A. Butman, R. Diaz-Arrastia, J. M. Gill, D. L. Brody, H. Zetterberg, K. Blennow and L. Chan (2020). “Neurofilament light as a biomarker in traumatic brain injury.” Neurology 95(6): e610-e622.
Shrank, W. H., N. A. DeParle, S. Gottlieb, S. H. Jain, P. Orszag, B. W. Powers and G. R. Wilensky (2021). “Health Costs And Financing: Challenges And Strategies For A New Administration.” Health Aff (Millwood) 40(2): 235-242.
Simon, D. W., M. J. McGeachy, H. Bayir, R. S. B. Clark, D. J. Loane and P. M. Kochanek (2017). “The far-reaching scope of neuroinflammation after traumatic brain injury.” Nat Rev Neurol 13(9): 572.
Thijssen, E. H., R. La Joie, A. Strom, C. Fonseca, L. Iaccarino, A. Wolf, S. Spina, I. E. Allen, Y. Cobigo, H. Heuer, L. Vande Vrede, N. K. Proctor, A. L. Lago, S. Baker, R. Sivasankaran, A. Kieloch, A. Kinhikar, L. Yu, M. A. Valentin, A. Jeromin, H. Zetterberg, O. Hansson, N. Mattsson-Carlgren, D. Graham, K. Blennow, J. H. Kramer, L. T. Grinberg, W. W. Seeley, H. Rosen, B. F. Boeve, B. L. Miller, C. E. Teunissen, G. D. Rabinovici, J. C. Rojas, J. L. Dage, A. L. Boxer, R. Advancing and i. Treatment for Frontotemporal Lobar Degeneration (2021). “Plasma phosphorylated tau 217 and phosphorylated tau 181 as biomarkers in Alzheimer's disease and frontotemporal lobar degeneration: a retrospective diagnostic performance study.” Lancet Neurol 20(9): 739-752.
Wang, K. K. W., F. H. Kobeissy, Z. Shakkour and J. A. Tyndall (2021). “Thorough overview of ubiquitin C-terminal hydrolase-L1 and glial fibrillary acidic protein as tandem biomarkers recently cleared by US Food and Drug Administration for the evaluation of intracranial injuries among patients with traumatic brain injury.” Acute Med Surg 8(1): e622.
Yuan, A. and R. A. Nixon (2021). “Neurofilament Proteins as Biomarkers to Monitor Neurological Diseases and the Efficacy of Therapies.” Front Neurosci 15: 689938.
Duan, Y., Dong, S., Gu, F., Hu, Y. & Zhao, Z. Advances in the pathogenesis of Alzheimer's disease: focusing on tau-mediated neurodegeneration. Transl Neurodegener 1, 24, doi:10.1186/2047-9158-1-24 (2012).
Lee, P. C., Bordelon, Y., Bronstein, J. & Ritz, B. Traumatic brain injury, paraquat exposure, and their relationship to Parkinson disease. Neurology 79, 2061-2066, doi:10.1212/WNL.0b013e3182749f28 (2012).
Sivanandam, T. M. & Thakur, M. K. Traumatic brain injury: a risk factor for Alzheimer's disease. Neurosci Biobehav Rev 36, 1376-1381, doi:10.1016/j.neubiorev.2012.02.013 (2012).
McKee, A. C. et al. Chronic traumatic encephalopathy in athletes: progressive tauopathy after repetitive head injury. J Neuropathol Exp Neurol 68, 709-735, doi:10.1097/NEN.0b013e3181a9d503 (2009).
Omalu, B. I., Hamilton, R. L., Kamboh, M. I., DeKosky, S. T. & Bailes, J. Chronic traumatic encephalopathy (CTE) in a National Football League Player: Case report and emerging medicolegal practice questions. J Forensic Nurs 6, 40-46, doi:10.1111/j.1939-3938.2009.01064.x (2010).
Peschl, Patrick et al. “Myelin Oligodendrocyte Glycoprotein: Deciphering a Target in Inflammatory Demyelinating Diseases.” Frontiers in immunology vol. 8 529. 8 May 2017, doi:10.3389/fimmu.2017.00529
Ambrosius W, Michalak S, et al. Myelin Oligodendrocyte Glycoprotein Antibody-Associated Disease: Current Insights into the Disease Pathophysiology, Diagnosis and Management. Int J Mol Sci. 2020 Dec. 24; 22(1):100. doi: 10.3390/ijms22010100. PMID: 33374173; PMCID: PMC7795410.
Marignier R, Hacohen Y, et al. Myelin-oligodendrocyte glycoprotein antibody-associated disease. Lancet Neurol. 2021 September; 20(9):762-772. doi: 10.1016/S1474-4422(21) 00218-0. Erratum in: Lancet Neurol. 2021 October; 20(10):e6. PMID: 34418402.
Zhang Z, Zoltewicz J S, et al. Wang K. K. W. (2014) Human Traumatic Brain Injury Induces Autoantibody Response against Glial Fibrillary Acidic Protein and Its Breakdown Products. PLoS One. 2014 Mar. 25; 9(3):e92698

Patent documents and publications mentioned in the specification are indicative of the levels of those skilled in the art to which the invention pertains. These documents and publications are incorporated herein by reference to the same extent as if each individual document or publication was specifically and individually incorporated herein by reference.

The foregoing description is illustrative of particular embodiments of the invention but is not meant to be a limitation upon the practice thereof. The following claims, including all equivalents thereof, are intended to define the scope of the invention.

The publications referenced are indicative of the levels of those skilled in the art to which the invention pertains. These publications are herein incorporated by reference to the same extent as if each individual publication was specifically and individually indicated to be incorporated by reference.

1. A process for determining an extent of a central nervous system (CNS) specific neurological condition in a subject comprising:
collecting a biological sample of biofluid from the subject; and
measuring a quantity of a first biomarker, or metabolite of or mRNA corresponding to, said first biomarker from said sample from saliva, a dried spot or through a microfluidic device.

2. The process of claim 1 wherein determining the extent of a neurological condition includes determining any of a magnitude, composite score of biomarker levels, burden, load, severity, phase of disease, disease course, or a combination thereof, with statistical, machine learning, or other methods.

3. The process of claim 1 wherein the quantity of said first biomarker is measured from the dried spot or dried saliva.

4. The process of claim 1 wherein said first biomarker is a protein or autoantibody, or a metabolite of or mRNA corresponding to said first biomarker not detectable or detected at a level below the quantity of absent the neurological condition.

5. The process of claim 1 further comprising measuring a quantity of a second biomarker in a biological sample obtained from the biofluid, or venous blood or arterial blood, from the subject to further determine the extent of the neurological condition, wherein said second biomarker is one of MOG, Anti-MOG autoantibody IgG, Anti-MOG autoantibody IgM, GFAP (glial fibrillary acidic protein; molecule weight 50K); GFAP breakdown products (GBDPs) of 38K (GBDP38K), of 44K (GBDP44K) and of a range of molecular wright in between 38K to 49K (GBDP38K-49K); NF-L (neurofilament protein-light), NFL-M (neurofilament-protein-Medium), NF-H (neurofilament protein-Heavy), pNF-H (phospho-NF-H), alpha-internexin- (NEF5); T-Tau (total Tau), P-Tau (phospho-Tau), P-Tau:T-Tau ratio, P-Tau (231), P-Tau (396/404), P-Tau (181, P-Tau (S202), P-Tau (217), OMG (oligodendrocyte myelin glycoprotein), MBP (myelin basic protein), MAG (myelin associated glycoprotein, Anti-MAG autoantibody IgG, Anti-MAG autoantibody IgM, Anti-GFAP autoantibody IgG, Anti-GFAP autoantibody IgM, Synapsin-1, -2, -3, VILIP-1 (Visinin-like 1), VILIP-3 (Visinin-like 3), UCH-L1 (ubiquitin C-terminal hydrolase-L1); alpha II-spectrin breakdown products (SBDP): SBDBP150N, SBDP150, SBDP145, SBDP150i, SBDP120; MAP2 (microtubule associated protein-2), MAP6 (microtubule associated protein-6); Vimentin; WASF1 (WASP Family Member 1), WASF3 (WASP Family Member 3), VAMP5 (Vesicle Associated Membrane Protein 5), VAMP2 (Synaptobrevin; Vesicle Associated Membrane Protein 2), SNAP25 (Synaptosome Associated Protein 25), SNAP23 (Synaptosome Associated Protein 23), BDNF (Brain derived neurotrophic factor), ProBDNF (Pro-Brain derived neurotrophic factor), CAMKK1, CAMK-II (Calcium/Calmodulin Dependent Protein Kinase II), COL4A3BP, CERT1 (Ceramide transfer protein (CERT1)), DUSP3 (Dual Specificity Phosphatase 3), TBCB (Tubulin folding co-factor B), Ninj-1 (Ninjurin-1), HMGB-1 (High mobility group box 1), SAA (Serum amyloid A), C-RP (C-reactive protein), C-fibronectin, VEGF-A (vascular endothelial growth factor-A), VEGF-C(vascular endothelial growth factor-C), MCP-4 (monocyte chemoattractant protein-4), Eotaxin-3, sCD30 (soluble CD30 glycoprotein), ITAC (CXCL11; Interferon-inducible T-cell alpha chemoattractant), sICAM1 (Soluble intercellular adhesion molecule-1), IL-6 (interleukin-6), IL-15 (interleukin-15), PDGF-A (Platelet-derived growth factor-A), IMPA1 (Inositol Monophosphatase 1), ZBTB16 (Zinc Finger And BTB Domain Containing 16), PRDX6 (Peroxiredoxin 6), NFATC1 (N-Alpha-Acetyltransferase 10), NAA10 (N-Alpha-Acetyltransferase 10), ING1 (Inhibitor Of Growth Family Member 1), BCR (Breakpoint cluster region protein), DCTN2 (Dinectin-2), FHIT (Fragile Histidine Triad Diadenosine Triphosphatase), MAP2K6 (Dual specificity mitogen-activated protein kinase kinase 6), METAP1D (Methionyl Aminopeptidase Type 1D), NAA10 (N-alpha-acetyltransferase 10, NatA), SULT2A1 (Sulfotransferase 2A1), ZBTB16 (Zinc finger and BTB domain-containing protein 16), ARHGEF12 (Rho Guanine Nucleotide Exchange Factor 12) and TACC3 (Transforming acidic coiled-coil-containing protein 3), Amyloid β-Peptide (1-40), Amyloid β-Peptide (1-42), SV2A (Synaptic Vesicle Glycoprotein 2A), SV2B (Synaptic Vesicle Glycoprotein 2B), SV2C (Synaptic Vesicle Glycoprotein 2C), apolipoprotein E4 (APOE4), amyloid-β peptide (1-40 and 1-42 and their ratio), adenosine, NAA, NAAG, norepinephrine, myoinositol, glutamate, glutamine, or a combination thereof.

6. The process of claim 1 wherein the quantity of said first biomarker correlated, via a correction factor to an amount of said first biomarker obtained from a second biological sample from the subject, the biological samples being cerebrospinal fluid, tears, saliva, sweat, breath, urine, venous or arterial whole blood, a fraction of the whole blood, venous or arterial serum, venous or arterial plasma, body tissue or tissue lysate, or a dried spot derived from any of the aforementioned.

7. (canceled)

8. (canceled)

9. The process of claim 1 wherein said first biomarker is one of MOG, Neurofilament L, Tau, glial fibrillary acidic protein (GFAP) or a GFAP breakdown product.

10. The process of claim 5 wherein said first biomarker is one of Neurofilament L, Tau, glial fibrillary acidic protein (GFAP) or a GFAP breakdown product and said second biomarker is another of Neurofilament-L, Tau, glial fibrillary acidic protein (GFAP) or the GFAP breakdown product.

11. (canceled)

12. (canceled)

13. (canceled)

14. (canceled)

15. The process of claim 1 wherein said CNS specific condition is Traumatic brain injury (TBI, including: mild traumatic brain injury, concussion, moderate traumatic brain injury, or severe traumatic brain injury), Multiple sclerosis (MS, including: clinically isolated syndrome, relapsing remitting multiple sclerosis, secondary progressive multiple sclerosis, and primary progressive multiple sclerosis), Brain metastatic breast cancer (bmBC), Post-traumatic epilepsy (PTE), Alzheimer's disease (AD, including: preclinical Alzheimer's disease, mild cognitive impairment, prodromal Alzheimer's disease, and various dementias), Alzheimer's disease-related dementias (ADRD), Chronic traumatic encephalopathy (CTE), Frontotemporal degeneration (FTD), Spinal cord injury (SCI), COVID-19, or Alexander disease (AxD).

16. The process of claim 1 further comprising administering a compound, imaging agent, or drug including a small molecule or large molecule such as a biologic, antibody, fusion protein, virus, cell, or antibody conjugate or non-pharmacological therapy or intervention to said subject prior to or after said measuring begins.

17. The process of claim 5 wherein said quantity of said first biomarker and said quantity of said second biomarker are measured from the same biological sample.

18. A process for determining the magnitude of multiple sclerosis (MS), post-traumatic epilepsy (PTE), Alexander's disease (AxD), Alzheimer's disease (AD), or traumatic brain injury (TBI) in a subject comprising: measuring at least two biomarkers of: a quantity of myelin oligodendrocyte glycoprotein (MOG), Anti-MOG autoantibody IgG, or Anti-MOG autoantibody IgM, a quantity of NFL, a quantity of GFAP, a quantity of a GFAP breakdown product, and a quantity of Tau in one or more biofluid biological samples obtained from said subject at a first time to determine a result of an extent or phenotype of multiple sclerosis, post-traumatic epilepsy, Alexander's disease (AxD), Alzheimer's disease (AD), or traumatic brain injury (TBI) in said subject.

19. The process of claim 18 comprising measuring at least four of the aforementioned.

20. (canceled)

21. The process of claim 18 wherein said quantity of said NFL, GFAP or both are measured at the same time as said MOG or said MOG antibody.

22. (canceled)

23. (canceled)

24. (canceled)

25. The process of claim 18 further comprising administering a compound, imaging agent, or drug including a small molecule or large molecule such as a biologic, antibody, fusion protein, virus, cell, or antibody conjugate or non-pharmacological therapy or intervention to said subject prior to or after said measuring begins.

26. (canceled)

27. The process of claim 18 further comprising measuring a combination score that is the sum of standardized or non-standardized quantity of the MOG with a quantity of a second and third or more biomarker selected from GFAP, NFL, VEGF-A, P-Tau, T-Tau and P-Tau:T-Tau ratio.

28. The process of claim 18 further comprising measuring a combination score that is the sum of standardized or non-standardized quantity of the at least two biomarkers with the quantity of GFAP (glial fibrillary acidic protein; molecule weight 50K); the GFAP breakdown products (GBDPs) of 38K (GBDP38K), or 44K (GBDP44K) or of a range of molecular wright in between 38K to 49K (GBDP38K-49K); or a second and third or more biomarker selected from NFL-L (neurofilament protein-light), NFL-M (neurofilament-protein-Medium), NF-H (neurofilament protein-Heavy), pNF-H (phospho-NF-H), alpha-internexin-(NEF5); T-Tau (total Tau), P-Tau (phospho-Tau), P-Tau:T-Tau ratio, P-Tau (231), P-Tau (396/404), P-Tau (181), P-Tau (S202), P-Tau (217), OMG (oligodendrocyte myelin glycoprotein), MBP (myelin basic protein), MAG (myelin associated glycoprotein, Anti-MAG autoantibody IgG, Anti-MAG autoantibody IgM, Anti-GFAP autoantibody IgG, Anti-GFAP autoantibody IgM, Synapsin-1, -2, -3, VILIP (Visinin-like 1), VILIP (Visinin-like 3), UCH-L1 (ubiquitin C-terminal hydrolase-1); alpha II-spectrin breakdown products (SBDP): SBDBP150N, SBDP150, SBDP145, SBDP150i, SBDP120; MAP2 (microtubule associated protein-2), MAP6 (microtubule associated protein-6); Vimentin; WASF1 (WASP Family Member 1), WASF3 (WASP Family Member 3), VAMP5 (Vesicle Associated Membrane Protein 5), VAMP2 (Synaptobrevin; Vesicle Associated Membrane Protein 2), SNAP25 (Synaptosome Associated Protein 25), SNAP23 (Synaptosome Associated Protein 23), BDNF (Pro-Brain derived neurotrophic factor), ProBDNF (Pro-Brain derived neurotrophic factor), CAMKK1, CAMKII (Calcium/Calmodulin Dependent Protein Kinase II), COL4A3BP, CERT1 (Ceramide transfer protein (CERT1)), DUSP3 (Dual Specificity Phosphatase 3), TBCB (Tubulin folding co-factor B), Ninj-1 (Ninjurin-1), HMGB-1 (High mobility group box 1), SAA (Serum amyloid A), C-RP (C-reactive protein), C-fibronectin, VEGF-A (vascular endothelial growth factor-A), VEGF-C (vascular endothelial growth factor-C), MCP-4 (monocyte chemoattractant protein-4), Eotaxin-3, sCD30 (soluble CD30 glycoprotein), ITAC (CXCL11; Interferon-inducible T-cell alpha chemoattractant), sICAM1 (Soluble intercellular adhesion molecule-1), IL-6 (interleukin-6), IL-15 (interleukin-15), PDGF-A (Platelet-derived growth factor-A), IMPA1 (Inositol Monophosphatase 1), ZBTB16 (Zinc Finger And BTB Domain Containing 16), PRDX6 (Peroxiredoxin 6), NFATC1 (N-Alpha-Acetyltransferase 10), NAA10 (N-Alpha-Acetyltransferase 10), ING1 (Inhibitor Of Growth Family Member 1), BCR (Breakpoint cluster region protein), DCTN2 (Dinectin-2), FHIT (FHIT (Fragile Histidine Triad Diadenosine Triphosphatase), MAP2K6 (Dual specificity mitogen-activated protein kinase kinase 6), METAP1D (Methionyl Aminopeptidase Type 1D) NAA10 (N-alpha-acetyltransferase 10, NatA); SULT2A1 (Sulfotransferase 2A1), ZBTB16 (Zinc finger and BTB domain-containing protein 16), ARHGEF12 (Rho Guanine Nucleotide Exchange Factor 12) and TACC3 (Transforming acidic coiled-coil-containing protein 3), Amyloid β-Peptide (1-40), Amyloid β-Peptide (1-42), SV2A (Synaptic Vesicle Glycoprotein 2A), SV2B (Synaptic Vesicle Glycoprotein 2B), SV2C (Synaptic Vesicle Glycoprotein 2C), apolipoprotein E4 (APOE4), amyloid-β peptide (1-40 and 1-42 and their ratio), adenosine, NAA, NAAG, norepinephrine, glutamate, glutamine, myoinositol, or a combination thereof.

29. (canceled)

30. (canceled)

31. (canceled)

32. The process of claim 1 wherein said biomarker is detected from said metabolite of or said mRNA corresponding thereto.

33. A process for determining the magnitude of Alzheimer's disease (AD), multiple sclerosis (MS), Alexander disease (AxD), post-traumatic epilepsy (PTE), or traumatic brain injury (TBI) in a subject comprising: measuring a ratio of quantities of P-Tau:Tau, and at least one of a quantity of MOG, Anti-MOG autoantibody IgG, or Anti-MOG autoantibody IgM, a quantity of NFL, a quantity of GFAP or break down product thereof, a quantity of MBP, a quantity of an interleukin, in one or more biofluid biological samples obtained from said subject at a first time to determine a result of an extent of Alzheimer's disease (AD), multiple sclerosis (MS), Alexander disease (AxD), Post-Traumatic Epilepsy (PTE), or traumatic brain injury (TBI) in said subject.

34. (canceled)

35. (canceled)

36. (canceled)

37. (canceled)

38. The process of claim 33 further comprising administering a compound, imaging agent, or drug including a small molecule or large molecule such as a biologic, antibody, fusion protein, virus, cell, or antibody conjugate or non-pharmacological therapy or intervention to said subject prior to or after said measuring begins.

39. (canceled)

40. (canceled)

41. (canceled)
